(19) United States
(12) Patent Application Publication
(10) Pub. No.: US 2011/0263588 A1
(43) Pub. Date: Oct. 27, 2011

(54) TRICYCLIC COMPOUNDS AS GLUTAMATE RECEPTOR MODULATORS

(75) Inventors: Peter Bertinato, Middlesex (GB); Merav Fichman, Middlesex (GB); Shomir Ghosh, Middlesex (GB); Jian Lin, Middlesex (GB); Dalia Segal, Middlesex (GB); Zhaoda Zhang, Middlesex (GB)

(73) Assignee: GLAXO GROUP LIMITED, Greenford, Middlesex (GB)

(21) Appl. No.: 13/126,118

(22) PCT Filed: Oct. 23, 2009
(86) PCT No.: PCT/EP2009/064015

§ 371 Date: Jul. 12, 2011

Related U.S. Application Data
(60) Provisional application No. 61/108,666, filed on Oct. 27, 2008.

(51) Int. Cl. A61K 31/5377; C07D 471/04; A61K 31/4162; A61K 31/4439; A61K 31/437; A61K 31/454; A61P 25/28; A61P 25/14; A61P 25/22; A61P 25/24; A61P 29/00; A61P 25/06; A61P 1/04; A61P 25/34; A61P 25/00; C07D 491/052

(52) U.S. Cl. 514/230.5; 548/359.5; 546/275.7; 544/105; 546/82; 546/199; 514/406; 514/338; 514/293; 514/322

(57) ABSTRACT

The present invention relates to compounds that may be negative allosteric modulators of metabotropic receptors-subtype 5, and methods of making and using same.

### BACKGROUND

The amino acids glutamate and aspartate are known to function as excitator neurotransmitters, which activate a large series of receptors known as glutamate receptors. Glutamate receptors comprise a large family of proteins with extensive heterogeneity at the molecular level. At least three classes of glutamate receptors have been identified. One of these, the NMDA class of receptors, is specifically activated by the glutamate analog methyl-D-aspartate. When stimulated, NMDA receptors open ion channels and allow an influx of cations into a neuron. The non-NMDA receptors comprise another group of ionotropic receptors which mediate cation flow into neurons.

A third class of glutamate receptors are known as the metabotropic glutamate receptors (“mGluR”). To date, eight subtypes of mGluR have been cloned and classified into three groups on the basis of sequence similarities and pharmacological properties. These receptors are referred to as “metabotropic” because their stimulation does not appear to result directly in the opening of an ion channel, as is the case with the NMDA and non-NMDA ionotropic glutamate receptors. Instead, the cellular effects caused by the stimulation of a metabotropic receptor are mediated by G-proteins.

Modulation of metabotropic glutamate receptor subtype 5 (mGluR5) appears to affect affect the nervous system. For example, recent evidence demonstrates the involvement of mGluR5 in nociceptive processes and therefore modulation of mGluR5 using mGluR5-selective compounds is useful in the treatment of various pain states, including acute, persistent and chronic pain and neuropathic pain.

Modulators of mGluR5 may be also useful in the treatment of psychiatric and neurological disorders. For example, mGluR5-selective compounds appear effective in animal models of mood disorders, including anxiety and depression. Gene expression data from humans has appeared to indicate that modulation of mGluR5 may be useful for the treatment of schizophrenia. Studies have also shown a role for mGluR5, and the potential utility of mGluR5-modulatory compounds, in the treatment of movement disorders such as Parkinson's disease, and other research supports a role for mGluR5 modulation in the treatment of cognitive dysfunction, epilepsy, and neuroprotection. Studies with mGluR5 knockout mice and MPEP also suggest that modulation of these receptors may be useful in the treatment of drug addiction, drug abuse and drug withdrawal.

Accordingly, compounds and methods for modulating the activity of mGluR5 are needed to address the treatment of these and other diseases and conditions.

### SUMMARY

This disclosure is generally directed to compounds which may be negative allosteric modulators of metabotropic receptors-subtype 5, and their use as, for example, medicinal agents. Also provided are pharmaceutical compositions comprising at least one disclosed compound and a pharmaceutically acceptable carrier.

In an embodiment, provided herein are compounds represented by formula VII, and compositions comprising such compounds. Also provided herein are methods of treating disorders such as Alzheimer's disease, Fragile X syndrome, L-DOPA induced dyskinesia, depression, anxiety, migraine, pain, gastroesophageal reflux disease, and/or aiding smoking cessation comprising administering to a subject in need thereof a therapeutically effective amount of a compound of formula VII:

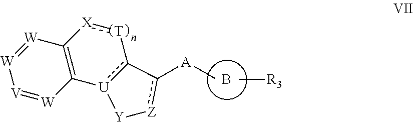

and pharmaceutically acceptable salts or N-oxides thereof, wherein:

each W is independently N or CR₁;

V is N or CR₂;

X is selected from the group consisting of: O, S, SO₂, N, NR₄, CR₅, CR₅R₆, and CO;

each T is independently selected from the group consisting of: CO, CR₇R_{8,} and NR₉;

U is selected from the group consisting of: C, CR₁₀, and N;

Y is selected from the group consisting of: NR₁₁, O, and CR₁₂;

Z is selected from the group consisting of: N, NR₁₃, and O;

A is selected from the group consisting of: —CONR₁₆—, —NR₁₆CO—, —CONR₁₆—CR₁₄R₁₅—, —O—, —NR₁₆CONR₁₆—, —CR₁₄R₁₅—NR₁₆CO—, —CR₁₄R₁₅—O—, —CR₁₄R₁₅—NR₁₆—, —O—CR₁₄R₁₅—, —NR₁₆—CR₁₄R₁₅—, —NR_{16—,}

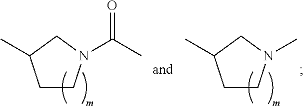

B is selected from the group consisting of: aryl, heteroaryl, and heterocyclyl; wherein B is optionally substituted by one, two, or three substituents each independently represented by R₃;

n is 0, 1 or 2;

m is 0, 1 or 2;

R₁, R₂, and R₃ are each independently selected from the group consisting of: alkoxy, alkyl, alkenyl, alkynyl, alkanoyl, amino, aryl, carboxamido, cycloalkyl, cyano, halogen, haloalkyl, heteroaryl, heterocyclyl, hydrogen, hydroxyl, nitro, sulfonamido, sulfonyl, and thio;

R₄, R₉, R₁₁, R₁₃, and R₁₆ are each independently selected from the group consisting of: alkyl, alkylsulfonyl, alkanoyl, cycloalkyl, haloalkyl, and hydrogen;

R₅, R₆, R₁₂, R₁₄, and R₁₅ are each independently selected from the group consisting of: alkoxy, alkyl, alkanoyl, amino, aryl, cycloalkyl, cyano, halogen, haloalkyl, hydrogen, and hydroxyl;

R₇ and R₈ are each independently selected from the group consisting of: alkoxy, alkyl, halogen, haloalkyl, hydrogen, and hydroxyl, or R₇ and R₈ taken together are oxo; and

R₁₀ is selected from the group consisting of: methyl, oxide, or hydrogen.

Treatment of diseases such as Parkinson's disease and panic disorder are also contemplated as described herein.

Another embodiment provides compounds represented by one or more of the following formulae:

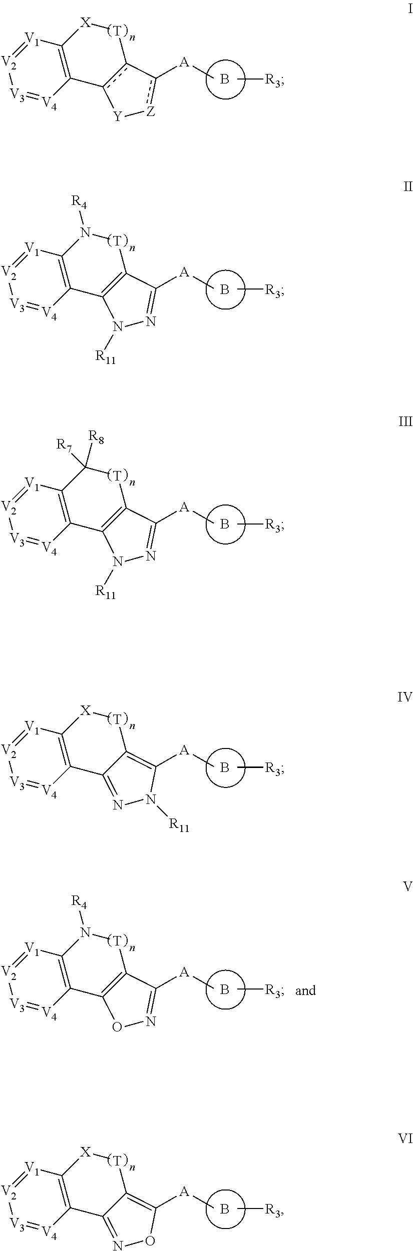

and pharmaceutically acceptable salts or N-oxides thereof, and in some embodiments, the substituents are defined above. Also contemplated herein are compositions that include a compound represented by one of formulas I, II, III, IV, V, and VI and e.g., a pharmaceutically acceptable excipient.

The disclosure further provides methods of modulating activity of one or more metabotropic receptors-subtype 5 comprising, for example, exposing said receptor to a disclosed compound. In one embodiment, the modulator compound is a negative allosteric modulator.

Also provided herein are methods of treating a disease associated with expression or activity of one or more metabotropic receptors-subtype 5 in a patient comprising administering to the patient a therapeutically effective amount of a disclosed compound. For example, provided herein are method of treating Alzheimer's disease, Fragile X syndrome, dyskinesia such as 1-dopa induced dyskinesia, depression, anxiety, migraine, pain, gastroesophageal reflux disease, and/or aiding smoking cessation comprising administering a compound represented by I, II, III, VI, V, VI, and/or VII. Also provided are compound represented by Formula VII for use in therapy and/or for the manufacture of a medicament for the treatment of disease associated with negative metabotropic receptors-subtype 5.

### DETAILED DESCRIPTION

The features and other details of the disclosure will now be more particularly described. Before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

### Definitions

“Treating” includes any effect, e.g., lessening, reducing, modulating, or eliminating, that results in the improvement of the condition, disease, disorder and the like.

The term “aldehyde” or “formyl” as used herein refers to the radical —CHO.

The term “alkanoyl” as used herein refers to a radical —O—CO-alkyl.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂alkenyl, C₂-C₁₀alkenyl, and C₂-C₆alkenyl, respectively. Exemplary alkenyl groups include, but are not limited to, vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl, etc.

The term “alkoxy” as used herein refers to an alkyl group attached to an oxygen (—O-alkyl-). Exemplary alkoxy groups include, but are not limited to, groups with an alkyl, alkenyl or alkynyl group of 1-12, 1-8, or 1-6 carbon atoms, referred to herein as C₁-C₁₂alkoxy, C₁-C₈alkoxy, and C₁-C₆alkoxy, respectively. Exemplary alkoxy groups include, but are not limited to methoxy, ethoxy, etc. Similarly, exemplary “alkenoxy” groups include, but are not limited to vinyloxy, allyloxy, butenoxy, etc.

The term “alkyl” as used herein refers to a saturated straight or branched hydrocarbon, such as a straight or branched group of 1-12, 1-10, or 1-6 carbon atoms, referred to herein as C₁-C₁₂alkyl, C₁-C₁₀alkyl, and C₁-C₆alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

Alkyl, alkenyl and alkynyl groups can optionally be substituted with or interrupted by at least one group selected from alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl and thiocarbonyl.

The term “alkynyl” as used herein refers to an unsaturated straight or branched hydrocarbon having at least one carbon-carbon triple bond, such as a straight or branched group of 2-12, 2-8, or 2-6 carbon atoms, referred to herein as C₂-C₁₂alkynyl, C₂-C₈alkynyl, and C₂-C₆alkynyl, respectively. Exemplary alkynyl groups include, but are not limited to, ethynyl, propynyl, butynyl, pentynyl, hexynyl, methylpropynyl, 4-methyl-1-butynyl, 4-propyl-2-pentynyl, and 4-butyl-2-hexynyl, etc.

The term “amide” or “amido” as used herein refers to a radical of the form —RₐC(O)N(R_{b})—, —RₐC(O)N(R_{b})R_{c}—, or —C(O)NR_{b}R_{c}, wherein R_{a,} R_{b} and R_{c} are each independently selected from alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, and nitro. The amide can be attached to another group through the carbon, the nitrogen, R_{b}, R_{c}, or Rₐ. The amide also may be cyclic, for example R_{b} and R_{c}, Rₐ and R_{b}, or Rₐ and R_{c} may be joined to form a 3- to 12-membered ring, such as a 3- to 10-membered ring or a 5- to 6-membered ring. The term “carboxamido” refers to the structure —C(O)NR_{b}R_{c}.

The term “amidino” as used herein refers to a radical of the form —C(═NR)NR′R″ where R, R′, and R″ can each independently be selected from alkyl, alkenyl, alkynyl, amide, aryl, arylalkyl, cyano, cycloalkyl, haloalkyl, heteroaryl, heterocyclyl, hydroxyl, ketone and nitro.

The term “amine” or “amino” as used herein refers to a radical of the form —NR_{d}Rₑ, —N(R_{d})Rₑ—, or —RₑN(R_{d})R_{f}— where R_{d}, Rₑ, and R_{f} are independently selected from alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, and nitro. The amino can be attached to the parent molecular group through the nitrogen, R_{d}, Rₑ or R_{f}. The amino also may be cyclic, for example any two of Rd, Re or Rf may be joined together or with the N to form a 3- to 12-membered ring, e.g., morpholino or piperidinyl. The term amino also includes the corresponding quaternary ammonium salt of any amino group, e.g., —[N(Rd)(Re)(Rf)]+. Exemplary amino groups include aminoalkyl groups, wherein at least one of R_{d}, Rₑ, or R_{f} is an alkyl group.

The term “aryl” as used herein refers to refers to a mono-, bi-, or other multi-carbocyclic, aromatic ring system. The aromatic ring may be substituted at one or more ring positions with substituents selected from alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl and thiocarbonyl. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, and/or aryls. Exemplary aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl.

The term “arylalkyl” as used herein refers to an aryl group having at least one alkyl substituent, e.g. -aryl-alkyl-. Exemplary arylalkyl groups include, but are not limited to, arylalkyls having a monocyclic aromatic ring system, wherein the ring comprises 6 carbon atoms. For example, “phenylalkyl” includes phenylC₄alkyl, benzyl, 1-phenylethyl, 2-phenylethyl, etc.

The term “azido” as used herein refers to the radical —N₃.

The term “carbamate” as used herein refers to a radical of the form —R_{g}OC(O)N(Rₕ)—, —R_{g}OC(O)N(Rₕ)Rᵢ—, or —OC(O)NRₕRᵢ, wherein R_{g,} Rₕ and Rᵢ are each independently selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, sulfide, sulfonyl, and sulfonamide. Exemplary carbamates include, but are not limited to, arylcarbamates or heteroaryl carbamates, e.g., wherein at least one of R_{g,} Rₕ and Rᵢ are independently selected from aryl or heteroaryl, such as phenyl and pyridinyl.

The term “carbonyl” as used herein refers to the radical —C(O)—.

The term “carboxamido” as used herein refers to the radical —C(O)NRR′, where R and R′ may be the same or different. R and R′ may be selected from, for example, alkyl, aryl, arylalkyl, cycloalkyl, formyl, haloalkyl, heteroaryl and heterocyclyl.

The term “carboxy” as used herein refers to the radical —COOH or its corresponding salts, e.g. —COONa, etc.

The term “cyano” as used herein refers to the radical —CN.

The term “cycloalkoxy” as used herein refers to a cycloalkyl group attached to an oxygen.

The term “cycloalkyl” as used herein refers to a monovalent saturated or unsaturated cyclic, bicyclic, or bridged bicyclic hydrocarbon group of 3-12, 3-8, 4-8, or 4-6 carbons, referred to herein, e.g., as “C₄₋₈cycloalkyl,” derived from a cycloalkane. Exemplary cycloalkyl groups include, but are not limited to, cyclohexanes, cyclohexenes, cyclopentanes, cyclopentenes, cyclobutanes and cyclopropanes. Cycloalkyl groups may be substituted with alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl and thiocarbonyl. Cycloalkyl groups can be fused to other cycloalkyl, aryl, or heterocyclyl groups.

The term “ether” refers to a radical having the structure —RₗO—Rₘ—, where Rₗ and Rₘ can independently be alkyl, aryl, cycloalkyl, heterocyclyl, or ether. The ether can be attached to the parent molecular group through Rₗ or Rₘ. Exemplary ethers include, but are not limited to, alkoxyalkyl and alkoxyaryl groups. Ether also includes polyethers, e.g., where one or both of Rₗ and Rₘ are ethers.

The terms “halo” or “halogen” or “Hal” as used herein refer to F, Cl, Br, or I.

The term “haloalkyl” as used herein refers to an alkyl group substituted with one or more halogen atoms.

The terms “heteroaryl” as used herein refers to a 5-15 membered mono-, bi-, or other multi-cyclic, aromatic ring system containing one or more heteroatoms, for example one to four heteroatoms, such as nitrogen, oxygen, and sulfur. Heteroaryls can also be fused to non-aromatic rings. The heteroaryl ring may be substituted at one or more positions with such substituents as described above, as for example, alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl and thiocarbonyl. Illustrative examples of heteroaryl groups include, but are not limited to, acridinyl, benzimidazolyl, benzofuryl, benzothiazolyl, benzothienyl, benzoxazolyl, carbazolyl, carbolinyl, cinnolinyl, furazanyl, furyl, imidazolyl, indazolyl, indolizinyl, indolyl, isobenzofuryl, isoindolyl, isoquinolinyl, isothiazolyl, isoxazolyl, naphthyridinyl, oxadiazolyl, oxazolyl, phenanthridinyl, phenanthrolinyl, phenarsazinyl, phenazinyl, phenothiazinyl, phenoxazinyl, phthalazinyl, pteridinyl, purinyl, pyrazinyl, pyrazolyl, pyrazyl, pyridazinyl, pyridinyl, pyrimidilyl, pyrimidyl, pyrrolyl, quinolinyl, quinolizinyl, quinoxalinyl, quinoxaloyl, quinazolinyl, tetrazolyl, thiadiazolyl, thianthrenyl, thiazolyl, thienyl, thiophenyl, triazinyl, (1,2,3,)- and (1,2,4)-triazolyl, and the like. Exemplary heteroaryl groups include, but are not limited to, a monocyclic aromatic ring, wherein the ring comprises 2 to 5 carbon atoms and 1 to 3 heteroatoms.

The terms “heterocyclyl” or “heterocyclic group” are art-recognized and refer to saturated or partially unsaturated 3- to 10-membered ring structures, alternatively 3- to 7-membered rings, whose ring structures include one to four heteroatoms, such as nitrogen, oxygen, and sulfur. Heterocycles may also be mono-, bi-, or other multi-cyclic ring systems. A heterocycle may be fused to one or more aryl, partially unsaturated, or saturated rings. Heterocyclyl groups include, for example, biotinyl, chromenyl, dihydrofuryl, dihydroindolyl, dihydropyranyl, dihydrothienyl, dithiazolyl, homopiperidinyl, imidazolidinyl, isoquinolyl, isothiazolidinyl, isoxazolidinyl, morpholinyl, oxolanyl, oxazolidinyl, phenoxanthenyl, piperazinyl, piperidinyl, pyranyl, pyrazolidinyl, pyrazolinyl, pyridyl, pyrimidinyl, pyrrolidinyl, pyrrolidin-2-onyl, pyrrolinyl, tetrahydrofuryl, tetrahydroisoquinolyl, tetrahydropyranyl, tetrahydroquinolyl, thiazolidinyl, thiolanyl, thiomorpholinyl, thiopyranyl, xanthenyl, lactones, lactams such as azetidinones and pyrrolidinones, sultams, sultones, and the like. The heterocyclic ring may be substituted at one or more positions with substituents such as alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl and thiocarbonyl.

The term “heterocycloalkyl” is art-recognized and refers to a saturated heterocyclyl group as defined above.

The term “heterocyclylalkoxy” as used herein refers to a heterocyclyl attached to an alkoxy group.

The term “heterocyclyloxyalkyl” refers to a heterocyclyl attached to an oxygen (—O—), which is attached to an alkyl group.

The terms “hydroxy” and “hydroxyl” as used herein refers to the radical —OH.

The term “hydroxyalkyl” as used herein refers to a hydroxy radical attached to an alkyl group.

The term “imino” as used herein refers to the radical —C(═N)—R″, where R″ can be, for example, alkyl, alkenyl, alkynyl, aryl, arylalkyl, cycloalkyl, ether, haloalkyl, heteroaryl, heterocyclyl, and ketone.

The term “nitro” as used herein refers to the radical —NO₂.

The term “phenyl” as used herein refers to a 6-membered carbocyclic aromatic ring. The phenyl group can also be fused to a cyclohexane or cyclopentane ring. Phenyl can be substituted with one or more substituents including alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl and thiocarbonyl.

The term “phosphate” as used herein refers to the radical —OP(O)(ORₐₐ)₂ or its anions. The term “phosphanato” refers to the radical —P(O)(ORₐₐ)₂ or its anions. The term “phosphinato” refers to the radical —PRₐₐ(O)(ORₐₐ) or its anion, where each Rₐₐ can be selected from, for example, alkyl, alkenyl, alkynyl, aryl, arylalkyl, cycloalkyl, hydrogen, haloalkyl, heteroaryl, and heterocyclyl.

The term “sulfate” as used herein refers to the radical —OS(O)(ORₐₐ)₂ or its anions, where Rₐₐ is defined above.

The term “sulfonamide” as used herein refers to a radical having the structure —N(Rᵣ)—S(O)₂—Rₛ— or —S(O)₂—N(Rᵣ)Rₛ, where Rᵣ, and Rₛ can be, for example, hydrogen, alkyl, aryl, cycloalkyl, and heterocyclyl. Exemplary sulfonamides include alkylsulfonamides (e.g., where Rₛ is alkyl), arylsulfonamides (e.g., where Rₛ is aryl), cycloalkyl sulfonamides (e.g., where Rₛ is cycloalkyl), and heterocyclyl sulfonamides (e.g., where Rₛ is heterocyclyl), etc.

The term “sulfonyl” as used herein refers to a radical having the structure RᵤSO₂—, where Rᵤ can be alkyl, aryl, cycloalkyl, and heterocyclyl, e.g., alkylsulfonyl. The term “alkylsulfonyl” as used herein refers to an alkyl group attached to a sulfonyl group.

The term “sulfide” as used herein refers to the radical having the structure R_{z}S—, where R_{z} can be alkoxy, alkyl, alkenyl, alkynyl, amide, amino, aryl, arylalkyl, carbamate, carboxy, cycloalkyl, ester, ether, formyl, haloalkyl, heteroaryl, heterocyclyl, and ketone. The term “alkylsulfide” as used herein refers to an alkyl group attached to a sulfur atom. Exemplary sulfides include “thio,” which as used herein refers to an —SH radical.

The term “thiocarbonyl” or “thiocarboxy” as used herein refers to compounds and moieties which contain a carbon connected with a double bond to a sulfur atom.

“Pharmaceutically or pharmacologically acceptable” include molecular entities and compositions that do not produce an adverse, allergic or other untoward reaction when administered to an animal, or a human, as appropriate. “For human administration, preparations should meet sterility, pyrogenicity, general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers.

“Individual,” “patient,” or “subject” are used interchangeably and include to any animal, including mammals, preferably mice, rats, other rodents, rabbits, dogs, cats, swine, cattle, sheep, horses, or primates, and most preferably humans. The compounds of the invention can be administered to a mammal, such as a human, but can also be other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated in the methods of the invention is desirably a mammal in whom modulation of metabotropic receptors-subtype 5 is desired. “Modulation” includes antagonism (e g., inhibition), agonism, partial antagonism and/or partial agonism.

In the present specification, the term “therapeutically effective amount” means the amount of the subject compound that will elicit the biological or medical response of a tissue, system, animal or human that is being sought by the researcher, veterinarian, medical doctor or other clinician. The compounds of the invention are administered in therapeutically effective amounts to treat a disease. Alternatively, a therapeutically effective amount of a compound is the quantity required to achieve a desired therapeutic and/or prophylactic effect, such as an amount which results in the prevention of or a decrease in the symptoms associated with a disease associated with metabotropic receptors-subtype 5.

The term “pharmaceutically acceptable salt(s)” as used herein refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly.

Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well known asymmetric synthetic methods.

Geometric isomers can also exist in the compounds of the present invention. The symbol [symbol] denotes a bond that may be a single, double or triple bond as described herein. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The compounds disclosed herein can exist in solvated as well as unsolvated forms with pharmaceutically acceptable solvents such as water, ethanol, and the like, and it is intended that the invention embrace both solvated and unsolvated forms. In one embodiment, the compound is amorphous. In one embodiment, the compound is a polymorph. In another embodiment, the compound is in a crystalline form.

The invention also embraces isotopically labeled compounds of the invention which are identical to those recited herein, except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F and ³⁶Cl, respectively.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the invention can generally be prepared by following procedures analogous to those disclosed in the e.g., Examples herein by substituting an isotopically labeled reagent for a non-isotopically labeled reagent.

The term “prodrug” refers to compounds that are transformed in vivo to yield a disclosed compound or a pharmaceutically acceptable salt, hydrate or solvate of the compound. The transformation may occur by various mechanisms, such as through hydrolysis in blood. For example, if a compound of the invention or a pharmaceutically acceptable salt, hydrate or solvate of the compound contains a carboxylic acid functional group, a prodrug can comprise an ester formed by the replacement of the hydrogen atom of the acid group with a group such as (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

Similarly, if a compound of the invention contains an alcohol functional group, a prodrug can be formed by the replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl(C₁-C₆)alkoxycarbonyloxymethyl, N—(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, —P(O)(O(_{C1}-C₆)alkyl)₂ or glycosyl (the radical resulting from the removal of a hydroxyl group of the hemiacetal form of a carbohydrate).

If a compound of the invention incorporates an amine functional group, a prodrug can be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl-natural α-aminoacyl, —C(OH)C(O)OY¹ wherein Y¹ is H, (C₁-C₆)alkyl or benzyl, —C(OY²)Y³ wherein Y² is (C₁-C₄)alkyl and Y³ is (C₁-C₆)alkyl, carboxy(C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino, morpholino, piperidin-1-yl or pyrrolidin-1-yl.

The disclosure provides, at least in part, compounds represented by formula VII, as depicted below. Also contemplated herein are compositions that include a compound represented by formula VII and e.g., a pharmaceutically acceptable carrier. In an embodiment, provided herein is a method of treating Alzheimer's disease, Fragile X syndrome, L-DOPA induced dyskinesia, depression, anxiety, migraine, pain, gastroesophageal reflux disease, and/or aiding smoking cessation comprising administering to a subject in need thereof a therapeutically effective amount of a compound of formula VII:

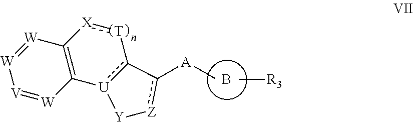

and pharmaceutically acceptable salts or N-oxides thereof, wherein:

each W is independently N or CR₁;

V is N or CR₂;

X is selected from the group consisting of: O, S, SO₂, N, NR₄, CR₅, CR₅R₆, and CO;

each T is independently selected from the group consisting of: CO, CR₇R_{8,} and NR₉;

U is selected from the group consisting of: C, CR₁₀, and N;

Y is selected from the group consisting of: N, NR₁₁, O, and CR₁₂;

Z is selected from the group consisting of: N, NR₁₃, and O;

A is selected from the group consisting of: —CONR₁₆—, —NR₁₆CO—, —O—, —CONR₁₆—CR₁₄R₁₅—, —O—, —NR₁₆CONR₁₆—, —CR₁₄R₁₅—NR₁₆CO—, —CR₁₄R₁₅—O—, —CR₁₄R₁₅—NR₁₆—, —O—CR₁₄R₁₅—, —NR₁₆—CR₁₄R₁₅—, —NR_{16—,}

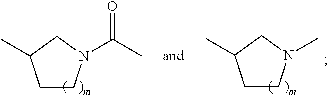

B is selected from the group consisting of: aryl, heteroaryl, and heterocyclyl; wherein B is optionally substituted by one, two, or three substituents each independently represented by R₃;

n is 0, 1 or 2;

m is 0, 1 or 2;

R₁, R₂, and R₃ are each independently selected from the group consisting of: alkoxy, alkyl, alkenyl, alkynyl, alkanoyl, amino, aryl, carboxamido, cycloalkyl, cyano, halogen, haloalkyl, heteroaryl, heterocyclyl, hydrogen, hydroxyl, nitro, sulfonamido, sulfonyl, and thio;

R₄, R₉, R₁₁, R₁₃, and R₁₆ are each independently selected from the group consisting of: alkyl, alkylsulfonyl, alkanoyl, cycloalkyl, haloalkyl, and hydrogen;

R₅, R₆, R₁₂, R₁₄, and R₁₅ are each independently selected from the group consisting of: alkoxy, alkyl, alkanoyl, amino, aryl, cycloalkyl, cyano, halogen, haloalkyl, hydrogen, and hydroxyl;

R₇ and R₈ are each independently selected from the group consisting of: alkoxy, alkyl, halogen, haloalkyl, hydrogen, and hydroxyl, or R₇ and R₈ taken together are oxo; or R₇ and R₈ taken together form a carbocycle or a heterocycle; and

R₁₀ is selected from the group consisting of: methyl, oxide, or hydrogen.

In one embodiment, B can be selected from:

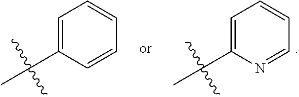

In some embodiments, one or two W are N, or each W and V are C. R₁ and R₂ may be, in some embodiments, each independently selected from the group consisting of: alkyl, halogen, and hydrogen, for example, R₁ and R₂ may be each independently selected from the group consisting of: chloro, fluoro, hydrogen, and methyl.

In some embodiments, R₁₁ is optionally substituted by one, two, three or more substituents selected from the group consisting of halogen, amino, nitro, hydroxyl, cyano, or alkoxy.

X, in certain embodiments, may be selected from the group consisting of: O, S, SO₂, N-alkanoyl, NH, NMe, CH₂, CF₂, CMe₂, CHOH, and CO. In another embodiment, T may selected from the group consisting of: CO, CH₂, CMe₂, NH, and NMe. The integer n, in a specific embodiment, may be 1 or 2. In an embodiment, U, Y, and Z may be chosen from one of the following groups:

A) U is C, Y is N-alkyl or NH, and Z is N;

B) U is C, Y is N, and Z is N-alkyl;

C) U is N, Y is CH or C-alkyl, and Z is N;

D) U is C, Y is O, and Z is N; and

E) U is C, Y is N, and Z is O.

For example, U may be C, Y is NMe, and Z is N.

In an embodiment, A may be selected from the group consisting of: —CONH—, —CONMe-, —CH₂—NH—, —CH₂—NEt-, —CMe₂-NH—, —CMe₂-O—, —CHMe-O—, —CHMe-NH—,

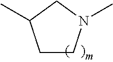

where m is 0, —NH—CHMe-, —O—CHMe-, —NH—, and —CONH—CH₂—, for example, A may be —CONH—.

In an embodiment, R₁, R₂, and R₃ are each independently selected from the group consisting of: bromo, carboxamido, chloro, chlorophenyl, cyano, cyclopropyl, diethoxyphenyl, diethylaminoethoxycarbonyl, diethylaminosulfonyl, ethoxycarbonyl, ethoxyphenyl, ethyl, ethylpiperazinylmethylphenyl, fluoro, fluorophenyl, hydrogen, isopropyl, methoxy, methoxycarbonyl, methyl, methylcarbonyl, methylpiperadinylmethylphenyl, methylpiperazinyl, methylthio, nitro, phenyl, piperidinylsulfonyl, sulfonamido, and trifluoromethyl

One embodiment provides a compound represented by formula I:

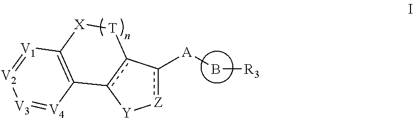

and pharmaceutically acceptable salts or N-oxides thereof; wherein
V₁, V₂, V₃, and V₄ are selected, independently for each occurrence, from the group consisting of: N and CR₂;
X is NR₄ or CR₇R₈;
Y and Z are each independently selected from the group consisting of: NR₁₁ and N;
T is CO or CR₇R₈, wherein at least one T is CR₇R_{8;}
n is 0, 1 or 2;
A is selected from the group consisting of: —CONR₁₆—, —NR₁₆CO—, —O—, —CONR₁₆—CR₁₄R₁₅—, —NR₁₆CONR₁₆—, —CR₁₄R₁₅—NR₁₆CO—, —CR₁₄R₁₅—O—, —CR₁₄R₁₅—NR₁₆—, —O—CR₁₄R₁₅—, —NR₁₆—CR₁₄R₁₅—, —NR₁₆—,

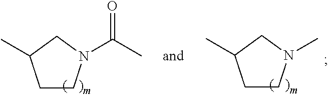

m is 0, 1 or 2;
R₂ is independently selected for each occurrence from the group consisting of: alkoxy, alkyl, alkenyl, alkynyl, alkanoyl, amino, aryl, carboxamido, cycloalkyl, cyano, halogen, haloalkyl, heteroaryl, heterocyclyl, hydrogen, hydroxyl, nitro, sulfonamido, sulfonyl, and thio;
B is selected from the group consisting of: aryl, heteroaryl, and heterocyclyl, wherein B is optionally substituted by one, two, or three substituents each independently represented by R₃;
R₃ is selected from the group consisting of: alkoxy, alkyl, alkenyl, alkynyl, alkanoyl, amino, aryl, carboxamido, cycloalkyl, cyano, halogen, haloalkyl, heteroaryl, heterocyclyl, hydrogen, hydroxyl, nitro, sulfonamido, sulfonyl, and thio;
R₄ is selected from the group consisting of: alkyl, alkylsulfonyl, alkanoyl, cycloalkyl, haloalkyl, and hydrogen;
R₇ and R₈ are each independently selected from the group consisting of: alkoxy, alkyl, halogen, haloalkyl, hydrogen and hydroxyl, or R₇ and R₈ taken together are oxo; or R₇ and R_{g} taken together form a carbocycle or a heterocycle;
R₁₁ is selected from the group consisting of: alkyl, cycloalkyl, or haloalkyl;
R₁₆ is selected from the group consisting of: alkyl, alkanoyl, cycloalkyl, haloalkyl, and hydrogen; and
R₁₄ and R₁₅ are each independently selected from the group consisting of: alkoxy, alkyl, alkanoyl, amino, aryl, cycloalkyl, cyano, halogen, haloalkyl, hydrogen, and hydroxyl.

In one embodiment, B can be selected from:

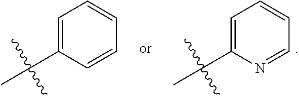

In one embodiment, each of V₁, V₂, V₃, and V₄ can be CR₂. In another embodiment, each of V₁, V₂, and V₃ can be CR_{2,} and V₄ can be N. In another exemplary embodiment, each of V₁, V₂, and V₄ is CR_{2,} and V₃ is N.

In another embodiment, R₁₁ is C₁₋₃alkyl and/or R₄ is selected from the group consisting of: alkyl, alkanoyl, and hydrogen. T, in some embodiments, may be CH₂ or CF₂. In some embodiments, R₁₁ is optionally substituted by one, two, three or more substituents selected from the group consisting of halogen, amino, nitro, hydroxyl, cyano, or alkoxy.

X, in some embodiments, may be X is NR₄ or may be CR₇R₈. In some embodiments, X is selected from the group consisting of: CH_{2,} CHOH, and CO. T is CH₂.

The integer n may be, in some embodiments, 1 or 2.

A, in some embodiments, may be for example —CONR₁₆— or —CR₁₄R₁₅—NR₁₆—, wherein, in a specific embodiment, R₁₆ may be hydrogen or methyl.

Another embodiment provides a compound represented by formula II:

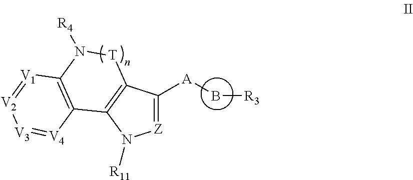

and pharmaceutically acceptable salts or N-oxides thereof; wherein
V₁, V₂, V₃, and V₄ are selected, independently for each occurrence, from the group consisting of: N and CR₂;
T is CO or CR₇R₈, wherein at least one T is CR₇R_{8;}
n is 0, 1 or 2;
A is selected from the group consisting of: —CONR₁₆—, —NR₁₆CO—, —O—, —CONR₁₆—CR₁₄R₁₅—, —NR₁₆CONR₁₆—, —CR₁₄R₁₅—NR₁₆CO—, —CR₁₄R₁₅—O—, —CR₁₄R₁₅—NR₁₆—, —O—CR₁₄R₁₅—, —NR₁₆—CR₁₄R₁₅—, —NR₁₆—,

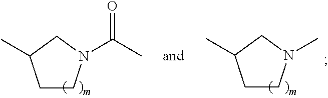

m is 0, 1 or 2;
R₂ is independently selected for each occurrence from the group consisting of: alkoxy, alkyl, alkenyl, alkynyl, alkanoyl, amino, aryl, carboxamido, cycloalkyl, cyano, halogen, haloalkyl, heteroaryl, heterocyclyl, hydrogen, hydroxyl, nitro, sulfonamido, sulfonyl, and thio;
B is selected from the group consisting of: aryl, heteroaryl, and heterocyclyl wherein B is optionally substituted by one, two, or three substituents each independently represented by R₃;
R₃ is selected from the group consisting of: alkoxy, alkyl, alkenyl, alkynyl, alkanoyl, amino, aryl, carboxamido, cycloalkyl, cyano, halogen, haloalkyl, heteroaryl, heterocyclyl, hydrogen, hydroxyl, nitro, sulfonamido, sulfonyl, and thio;
R₄ is selected from the group consisting of: alkyl, alkylsulfonyl, alkanoyl, cycloalkyl, haloalkyl, and hydrogen;
R₁₁ is selected from the group consisting of: alkyl, cycloalkyl, alkanoyl, aryl, heteroaryl, or heterocyclyl;
R₁₆ is selected from the group consisting of: alkyl, alkanoyl, cycloalkyl, haloalkyl, and hydrogen; and
R₁₄ and R₁₅ are each independently selected from the group consisting of: alkoxy, alkyl, alkanoyl, amino, aryl, cycloalkyl, cyano, halogen, haloalkyl, hydrogen, and hydroxyl.

In one embodiment, B can be selected from:

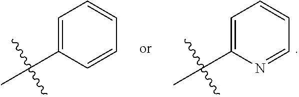

In one embodiment, each of V₁, V₂, V₃, and V₄ can be CR₂. In another embodiment, each of V₁, V₂, and V₃ can be CR_{2,} and V₄ can be N. In another exemplary embodiment, each of V₁, V₂, and V₄ is CR_{2,} and V₃ is N.

In another embodiment, R₁₁ is C₁₋₃alkyl and/or R₄ is selected from the group consisting of: alkyl, alkanoyl, and hydrogen. T, in some embodiments, may be CH₂ or CF₂. In some embodiments, R₁₁ is optionally substituted by one, two, three or more substituents selected from the group consisting of halogen, amino, nitro, hydroxyl, cyano, or alkoxy.

The integer n may be, in some embodiments, 1 or 2.

A, in some embodiments, may be for example —CONR₁₆— or —CR₁₄R₁₅—NR₁₆—, wherein, in a specific embodiment, R₁₆ may be hydrogen or methyl.

Another embodiment provides a compound represented by formula III:

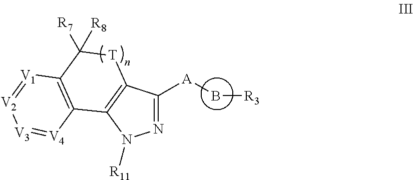

and pharmaceutically acceptable salts or N-oxides thereof; wherein
V₁, V₂, V₃, and V₄ are selected, independently for each occurrence, from the group consisting of: N and CR₂;
T is CO or CR₇R₈, wherein at least one T is CR₇R_{8;}
n is 0, 1 or 2;
A is selected from the group consisting of: —CONR₁₆—, —NR₁₆CO—, —O—, —CONR₁₆—CR₁₄R₁₅—, —NR₁₆CONR₁₆—, —CR₁₄R₁₅—NR₁₆CO—, —CR₁₄R₁₅—O—, —CR₁₄R₁₅—NR₁₆—, —O—CR₁₄R₁₅—, —NR₁₆—CR₁₄R₁₅—, —NR₁₆—,

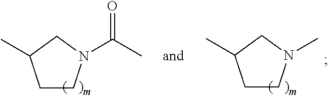

m is 0, 1 or 2;
R₂ is independently selected for each occurrence from the group consisting of: alkoxy, alkyl, alkenyl, alkynyl, alkanoyl, amino, aryl, carboxamido, cycloalkyl, cyano, halogen, haloalkyl, heteroaryl, heterocyclyl, hydrogen, hydroxyl, nitro, sulfonamido, sulfonyl, and thio;
B is selected from the group consisting of: aryl, heteroaryl, and heterocyclyl wherein B is optionally substituted by one, two, or three substituents each independently represented by R₃;
R₃ is selected from the group consisting of: alkoxy, alkyl, alkenyl, alkynyl, alkanoyl, amino, aryl, carboxamido, cycloalkyl, cyano, halogen, haloalkyl, heteroaryl, heterocyclyl, hydrogen, hydroxyl, nitro, sulfonamido, sulfonyl, and thio;
R₇ and R₈ are each independently selected from the group consisting of: alkyl, halogen, haloalkyl, and hydrogen; or R₇ and R₈ taken together form a carbocycle or a heterocycle;
R₁₁ is selected from the group consisting of: alkyl, cycloalkyl, alkanoyl, aryl, heteroaryl, or heterocyclyl;
R₁₆ is selected from the group consisting of: alkyl, alkanoyl, cycloalkyl, haloalkyl, and hydrogen; and
R₁₄ and R₁₅ are each independently selected from the group consisting of: alkoxy, alkyl, alkanoyl, amino, aryl, cycloalkyl, cyano, halogen, haloalkyl, hydrogen, and hydroxyl.

In one embodiment, B can be selected from:

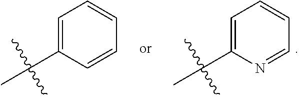

In one embodiment, each of V₁, V₂, V₃, and V₄ can be CR₂. In another embodiment, each of V₁, V₂, and V₃ can be CR_{2,} and V₄ can be N. In another exemplary embodiment, each of V₁, V₂, and V₄ is CR_{2,} and V₃ is N. In some embodiments, R₁₁ is optionally substituted by one, two, three or more substituents selected from the group consisting of halogen, amino, nitro, hydroxyl, cyano, or alkoxy.

In another embodiment, R₁₁ is C₁₋₃alkyl and/or R₄ is selected from the group consisting of: alkyl, alkanoyl, and hydrogen. T, in some embodiments, may be CH₂ or CF₂.

The integer n may be, in some embodiments, 1 or 2.

A, in some embodiments, may be for example —CONR₁₆— or —CR₁₄R₁₅—NR₁₆—, wherein, in a specific embodiment, R₁₆ may be hydrogen or methyl.

Another embodiment provides a represented by formula IV:

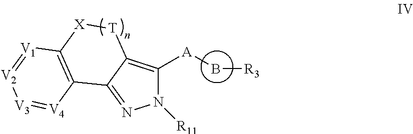

and pharmaceutically acceptable salts thereof; wherein
V₁, V₂, V₃, and V₄ are selected, independently for each occurrence, from the group consisting of: N and CR₂;
X is selected from the group consisting of: O, SO₂, NR₄ and CR₇R₈;
T is CO or CR₇R₈, wherein at least one T is CR₇R_{8;}
n is 0, 1 or 2;
A is selected from the group consisting of: —CONR₁₆—, —NR₁₆CO—, —O—, —CONR₁₆—CR₁₄R₁₅—, —NR₁₆CONR₁₆—, —CR₁₄R₁₅—NR₁₆CO—, —CR₁₄R₁₅—O—, —CR₁₄R₁₅—NR₁₆—, —O—CR₁₄R₁₅—, —NR₁₆—CR₁₄R₁₅—, —NR₁₆—,

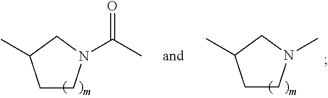

m is 0, 1 or 2;
R₂ is independently selected for each occurrence from the group consisting of: alkoxy, alkyl, alkenyl, alkynyl, alkanoyl, amino, aryl, carboxamido, cycloalkyl, cyano, halogen, haloalkyl, heteroaryl, heterocyclyl, hydrogen, hydroxyl, nitro, sulfonamido, sulfonyl, and thio;
B is selected from the group consisting of: aryl, heteroaryl, and heterocyclyl, wherein B is optionally substituted by one, two, or three substituents each independently represented by R₃;
R₃ is selected from the group consisting of: alkoxy, alkyl, alkenyl, alkynyl, alkanoyl, amino, aryl, carboxamido, cycloalkyl, cyano, halogen, haloalkyl, heteroaryl, heterocyclyl, hydrogen, hydroxyl, nitro, sulfonamido, sulfonyl, and thio;
R₄ is selected from the group consisting of: alkyl, alkylsulfonyl, alkanoyl, cycloalkyl, haloalkyl, and hydrogen;
R₇ and R₈ are each independently selected from the group consisting of: alkoxy, alkyl, halogen, haloalkyl, hydrogen and hydroxyl, or R₇ and R₈ taken together are oxo; or R₇ and R₈ taken together form a carbocycle or a heterocycle;
R₁₁ is selected from the group consisting of: alkyl, cycloalkyl, or haloalkyl;
R₁₆ is selected from the group consisting of: alkyl, alkanoyl, cycloalkyl, haloalkyl, and hydrogen; and
R₁₄ and R₁₅ are each independently selected from the group consisting of: alkoxy, alkyl, alkanoyl, amino, aryl, cycloalkyl, cyano, halogen, haloalkyl, hydrogen, and hydroxyl.

In one embodiment, B can be selected from:

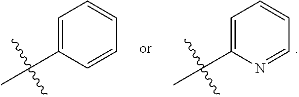

In one embodiment, each of V₁, V₂, V₃, and V₄ can be CR₂. In another embodiment, each of V₁, V₂, and V₃ can be CR_{2,} and V₄ can be N. In another exemplary embodiment, each of V₁, V₂, and V₄ is CR_{2,} and V₃ is N.

In another embodiment, R₁₁ is C₁₋₃alkyl and/or R₄ is selected from the group consisting of: alkyl, alkanoyl, and hydrogen. In some embodiments, R₁₁ is optionally substituted by one, two, three or more substituents selected from the group consisting of halogen, amino, nitro, hydroxyl, cyano, or alkoxy. T, in some embodiments, may be CH₂ or CF₂.

X, in some embodiments, may be X is NR₄ or may be CR₇R₈. In some embodiments, X is selected from the group consisting of: CH₂, CHOH, and CO. T is CH₂.

The integer n may be, in some embodiments, 1 or 2.

A, in some embodiments, may be for example —CONR₁₆— or —CR₁₄R₁₅—NR₁₆—, wherein, in a specific embodiment, R₁₆ may be hydrogen or methyl.

Another embodiment provides a compound represented by formula V:

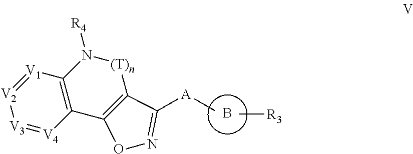

and pharmaceutically acceptable salts or N-oxides thereof; wherein
V₁, V₂, V₃, and V₄ are selected, independently for each occurrence, from the group consisting of: N and CR₂;
T is CO or CR₇R₈, wherein at least one T is CR₇R_{8;}
n is 0, 1 or 2;
A is selected from the group consisting of: —CONR₁₆—, —NR₁₆CO—, —O—, —CONR₁₆—CR₁₄R₁₅—, —NR₁₆CONR₁₆—, —CR₁₄R₁₅—NR₁₆CO—, —CR₁₄R₁₅—O—, —CR₁₄R₁₅—NR₁₆—, —O—CR₁₄R₁₅—, —NR₁₆—CR₁₄R₁₅—, —NR₁₆—,

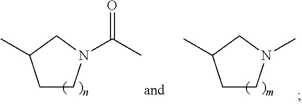

m is 0, 1 or 2;
R₂ is independently selected for each occurrence from the group consisting of: alkoxy, alkyl, alkenyl, alkynyl, alkanoyl, amino, aryl, carboxamido, cycloalkyl, cyano, halogen, haloalkyl, heteroaryl, heterocyclyl, hydrogen, hydroxyl, nitro, sulfonamido, sulfonyl, and thio;
B is selected from the group consisting of: aryl, heteroaryl, and heterocyclyl, optionally substituted by one, two or three substituents each independently represented by R₃;
R₃ is selected from the group consisting of: alkoxy, alkyl, alkenyl, alkynyl, alkanoyl, amino, aryl, carboxamido, cycloalkyl, cyano, halogen, haloalkyl, heteroaryl, heterocyclyl, hydrogen, hydroxyl, nitro, sulfonamido, sulfonyl, and thio;
R₄ is selected from the group consisting of: alkyl, alkylsulfonyl, alkanoyl, cycloalkyl, haloalkyl, and hydrogen;
R₇ and R₈ are each independently selected from the group consisting of: alkyl, halogen, haloalkyl, and hydrogen; or R₇ and R₈ taken together are oxo; or R₇ and R₈ taken together form a carbocycle or a heterocycle;
R₁₆ is selected from the group consisting of: alkyl, alkanoyl, cycloalkyl, haloalkyl, and hydrogen; and
R₁₄ and R₁₅ are each independently selected from the group consisting of: alkoxy, alkyl, alkanoyl, amino, aryl, cycloalkyl, cyano, halogen, haloalkyl, hydrogen, and hydroxyl.

In one embodiment, B can be selected from:

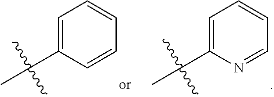

In one embodiment, each of V₁, V₂, V₃, and V₄ can be CR₂. In another embodiment, each of V₁, V₂, and V₃ can be CR_{2,} and V₄ can be N. In another exemplary embodiment, each of V₁, V₂, and V₄ is CR_{2,} and V₃ is N.

In another embodiment, R₄ is selected from the group consisting of: alkyl, alkanoyl, and hydrogen. T, in some embodiments, may be CH₂ or CF₂.

The integer n may be, in some embodiments, 1 or 2.

A, in some embodiments, may be for example —CONR₁₆— or —CR₁₄R₁₅—NR₁₆—, wherein, in a specific embodiment, R₁₆ may be hydrogen or methyl

Another claim embodiment provides a compound represented by formula VI:

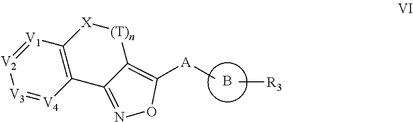

and pharmaceutically acceptable salts or N-oxides thereof; wherein
V₁, V₂, V₃, and V₄ are selected, independently for each occurrence, from the group consisting of: N and CR₂;
X is selected from the group consisting of: NR₄, S, SO₂, and CR₇R₈;
T is CO or CR₇R₈, wherein at least one T is CR₇R_{8;}
n is 0, 1 or 2;
A is selected from the group consisting of: —CONR₁₆—, —NR₁₆CO—, —O—, —CONR₁₆—CR₁₄R₁₅—, —NR₁₆CONR₁₆—, —CR₁₄R₁₅—NR₁₆CO—, —CR₁₄R₁₅—O—, —CR₁₄R₁₅—NR₁₆—, —O—CR₁₄R₁₅—, —NR₁₆—CR₁₄R₁₅—, —NR₁₆—,

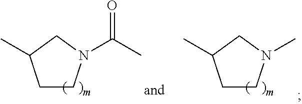

m is 0, 1 or 2;
R₂ is independently selected for each occurrence from the group consisting of: alkoxy, alkyl, alkenyl, alkynyl, alkanoyl, amino, aryl, carboxamido, cycloalkyl, cyano, halogen, haloalkyl, heteroaryl, heterocyclyl, hydrogen, hydroxyl, nitro, sulfonamido, sulfonyl, and thio;
B is selected from the group consisting of: aryl, heteroaryl, and heterocyclyl, wherein B is optionally substituted by one, two or three substituents each selected from R₃;
R₃ is selected from the group consisting of: alkoxy, alkyl, alkenyl, alkynyl, alkanoyl, amino, aryl, carboxamido, cycloalkyl, cyano, halogen, haloalkyl, heteroaryl, heterocyclyl, hydrogen, hydroxyl, nitro, sulfonamido, sulfonyl, and thio;
R₄ is selected from the group consisting of: alkyl, alkylsulfonyl, alkanoyl, cycloalkyl, haloalkyl, and hydrogen;
R₇ and R₈ are each independently selected from the group consisting of: alkyl, halogen, haloalkyl, and hydrogen, or R₇ and R₈ taken together are oxo; or R₇ and R₈ taken together form a carbocycle or a heterocycle;
R₁₁ is alkyl;
R₁₆ is selected from the group consisting of: alkyl, alkanoyl, cycloalkyl, haloalkyl, and hydrogen; and
R₁₄ and R₁₅ are each independently selected from the group consisting of: alkoxy, alkyl, alkanoyl, amino, aryl, cycloalkyl, cyano, halogen, haloalkyl, hydrogen, and hydroxyl.

In one embodiment, B can be selected from:

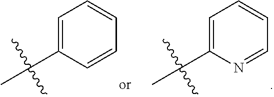

In one embodiment, each of V₁, V₂, V₃, and V₄ can be CR₂. In another embodiment, each of V₁, V₂, and V₃ can be CR_{2,} and V₄ can be N. In another exemplary embodiment, each of V₁, V₂, and V₄ is CR_{2,} and V₃ is N.

In another embodiment, R₁₁ is C₁₋₃alkyl and/or R₄ is selected from the group consisting of: alkyl, alkanoyl, and hydrogen. T, in some embodiments, may be CH₂ or CF₂.

X, in some embodiments, may be X is NR₄ or may be CR₇R₈. In some embodiments, X is selected from the group consisting of: CH_{2,} CHOH, and CO. The integer n may be, in some embodiments, 1 or 2.

A, in some embodiments, may be for example —CONR₁₆— or —CR₁₄R₁₅—NR₁₆—, wherein, in a specific embodiment, R₁₆ may be hydrogen or methyl.

Contemplated compounds, and pharmaceutical compositions, comprising at least one compound, may be selected from the group consisting of: N-(3-chlorophenyl)-1-methyl-4,5-dihydro-1H-benzo[g]indazole-3-carboxamide; 8-chloro-N-(3-chlorophenyl)-1-methyl-1,4-dihydrochromeno[4,3-c]pyrazole-3-carboxamide; N-(3-chlorophenyl)-8-fluoro-2-methyl-2,4-dihydrochromeno[4,3-c]pyrazole-3-carboxamide; N-(4-chloropyridin-2-yl)-1-methyl-1,4-dihydrochromeno[4,3-c]pyrazole-3-carboxamide; N-(3-chlorophenyl)-8-fluoro-1-methyl-1,4-dihydrochromeno[4,3-c]pyrazole-3-carboxamide; N-(3-chlorophenyl)-1,4,4-trimethyl-1,4-dihydrochromeno[4,3-c]pyrazole-3-carboxamide; 7-fluoro-1-methyl-N-(6-methylpyridin-2-yl)-1,4-dihydrochromeno[4,3-c]pyrazole-3-carboxamide; 3-chloro-N-((1-methyl-1,4-dihydrochromeno[4,3-c]pyrazol-3-yl)methyl)aniline; 1-methyl-N-(6-methylpyridin-2-yl)-4,5-dihydro-1H-pyrazolo[4,3-h]quinoline-3-carboxamide; N-(6-methylpyridin-2-yl)-1,4-dihydrochromeno[4,3-c]pyrazole-3-carboxamide; N-(4-cyanopyridin-2-yl)-1-methyl-1,4-dihydrochromeno[4,3-c]pyrazole-3-carboxamide; N-(2,3-dihydrobenzo[b][1,4]dioxin-6-yl)-1-methyl-1,4-dihydrochromeno[4,3-c]pyrazole-3-carboxamide; (2H-benzo[b][1,4]oxazin-4(3H)-yl)(1-methyl-1,4-dihydrochromeno[4,3-c]pyrazol-3-yl)methanone; 5-acetyl-N-(3-chlorophenyl)-1-methyl-4,5-dihydro-1H-pyrazolo[4,3-c]quinoline-3-carboxamide; N-(3-chlorophenyl)-1-methyl-1H-pyrazolo[4,3-c]quinoline-3-carboxamide; N-(3-chlorophenyl)-1-methyl-4,5-dihydro-1H-pyrazolo[4,3-c]quinoline-3-carboxamide; 5-bromo-N-ethyl-N-((1-methyl-1,4-dihydrochromeno[4,3-c]pyrazol-3-yl)methyl)furan-2-carboxamide; 1-isopropyl-N-(6-methylpyridin-2-yl)-4,5-dihydro-1H-pyrazolo[4,3-h]quinoline-3-carboxamide; 1-ethyl-N-(6-methylpyridin-2-yl)-4,5-dihydro-1H-pyrazolo[4,3-h]quinoline-3-carboxamide; 1-methyl-N-(6-methylpyridin-2-yl)-1,4,5,6,7,8-hexahydro-7,8-(2-pyridino)cyclohepta[c]pyrazole-3-carboxamide; 1,8-dimethyl-N-(6-methylpyridin-2-yl)-4,5-dihydro-1H-pyrazolo[4,3-h]quinoline-3-carboxamide; 1-methyl-N-(6-methylpyridin-2-yl)-4,5-dihydro-1H-pyrazolo[4,3-h]isoquinoline-3-carboxamide; 1-methyl-N-(6-methylpyridin-2-yl)-4,5-dihydro-1H-pyrazolo[3,4-f]quinoline-3-carboxamide; 1,5-dimethyl-N-(6-methylpyridin-2-yl)-1,4,5,6-tetrahydropyrazolo[4,3-c]pyrido[2,3-e]azepine-3-carboxamide; 1,6-dimethyl-N-(6-methylpyridin-2-yl)-1,4,5,6-tetrahydropyrazolo[3,4-d]pyrido[3,2-b]azepine-3-carboxamide; 1-methyl-N-(6-methylpyridin-2-yl)-6-oxo-1,4,5,6-tetrahydrobenzo[e]pyrazolo[4,3-c]azepine-3-carboxamide; 1-methyl-N-(6-methylpyridin-2-yl)-5-oxo-1,4,5,6-tetrahydrobenzo[b]pyrazolo[3,4-d]azepine-3-carboxamide; 5,5-difluoro-1-methyl-N-(6-methylpyridin-2-yl)-4,5-dihydro-1H-pyrazolo[4,3-h]quinoline-3-carboxamide; 5,5-difluoro-1-methyl-N-(6-methylpyridin-2-yl)-4,5-dihydro-1H-benzo[g]indazole-3-carboxamide; 1,6-dimethyl-N-(6-methylpyridin-2-yl)-1,4,5,6-tetrahydrobenzo[b]pyrazolo[3,4-d]azepine-3-carboxamide; 1,5-dimethyl-N-(6-methylpyridin-2-yl)-4,5-dihydro-1H-pyrazolo[4,3-c]quinoline-3-carboxamide; 8-fluoro-1,5-dimethyl-N-(6-methylpyridin-2-yl)-4,5-dihydro-1H-pyrazolo[4,3-c]quinoline-3-carboxamide; 1,5-dimethyl-N-(6-methylpyridin-2-yl)-1,4,5,6-tetrahydrobenzo[e]pyrazolo[4,3-c]azepine-3-carboxamide; 1-methyl-N-(6-methylpyridin-2-yl)-5-oxo-4,5-dihydro-1H-pyrazolo[4,3-h]quinoline-3-carboxamide; 1-methyl-N-(6-methylpyridin-2-yl)-4H-benzo[b]imidazo[1,5-d][1,4]oxazine-3-carboxamide; 1-methyl-N-(6-methylpyridin-2-yl)-5-oxo-4,5-dihydro-1H-pyrazolo[4,3-h]quinoline-3-carboxamide; 1-methyl-N-(6-methylpyridin-2-yl)-4H-benzo[b]imidazo[1,5-d][1,4]oxazine-3-carboxamide; 1-methyl-N-(6-methylpyridin-2-yl)-5-oxo-4,5-dihydro-1H-pyrazolo[4,3-c]isoquinoline-3-carboxamide; 1,5-dimethyl-N-(6-methylpyridin-2-yl)-4,5-dihydro-1H-pyrazolo[4,3-c][1,5]naphthyridine-3-carboxamide; 9-fluoro-1-methyl-N-(6-methylpyridin-2-yl)-6-oxo-1,4,5,6-tetrahydrobenzo[e]pyrazolo[4,3-c]azepine-3-carboxamide; 1,5,5-trimethyl-N-(6-methylpyridin-2-yl)-4,5-dihydro-1H-pyrazolo[4,3-h]quinoline-3-carboxamide; 1-methyl-N-(6-(trifluoromethyl)pyridin-2-yl)-4,5-dihydro-1H-pyrazolo[4,3-h]quinoline-3-carboxamide; 1-methyl-N-(6-methylpyridin-2-yl)-4,5-dihydro-1H-pyrazolo[3,4-f]quinoxaline-3-carboxamide; 9-fluoro-1-methyl-N-(6-methylpyridin-2-yl)-4,5-dihydro-1H-pyrazolo[3,4-f]quinoline-3-carboxamide; 8-fluoro-1-methyl-N-(6-methylpyridin-2-yl)-4,5-dihydro-1H-pyrazolo[3,4-f]quinoline-3-carboxamide; 7-fluoro-1-methyl-N-(6-methylpyridin-2-yl)-4,5-dihydro-1H-pyrazolo[4,3-h]quinoline-3-carboxamide; 6-fluoro-1-methyl-N-(6-methylpyridin-2-yl)-4,5-dihydro-1H-pyrazolo[4,3-h]quinoline-3-carboxamide; N-(6-methylpyridin-2-yl)-4H-benzo[b]imidazo[1,5-d][1,4]oxazine-3-carboxamide; N-(6-cyclopropylpyridin-2-yl)-1-methyl-4,5-dihydro-1H-pyrazolo[4,3-h]quinoline-3-carboxamide; 1-methyl-N-(1-methyl-5-phenyl-1H-pyrrol-2-yl)-4,5-dihydro-1H-pyrazolo[4,3-h]quinoline-3-carboxamide; 6-methyl-N-(2-(1-methyl-4,5-dihydro-1H-pyrazolo[4,3-h]quinolin-3-yl)propan-2-yl)pyridin-2-amine; 3-(2-(3-chlorophenoxy)propan-2-yl)-1-methyl-4,5-dihydro-1H-pyrazolo[4,3-h]quinoline; 6-methyl-N-(1-methyl-4,5-dihydro-1H-pyrazolo[4,3-h]quinolin-3-yl)picolinamide; 3-(1-(3-chlorophenoxy)ethyl)-1-methyl-4,5-dihydro-1H-pyrazolo[4,3-h]quinoline; 6-cyclopropyl-N-(1-(1-methyl-4,5-dihydro-1H-pyrazolo[4,3-h]quinolin-3-yl)ethyl)pyridin-2-amine; 1-methyl-N-(6-methylpyridin-2-yl)-1,4,5,6-tetrahydro-6,7-(2-pyridino)cyclopenta[c]pyrazole-3-carboxamide; 1-methyl-N-(2-methylpyrimidin-4-yl)-4,5-dihydro-1H-pyrazolo[4,3-h]quinoline-3-carboxamide; 1-methyl-N-(6-methylpyrazin-2-yl)-4,5-dihydro-1H-pyrazolo[4,3-h]quinoline-3-carboxamide; 9-fluoro-1-methyl-N-(6-methylpyridin-2-yl)-5-oxo-1,4,5,6-tetrahydrobenzo[b]pyrazolo[3,4-d]azepine-3-carboxamide; 1-methyl-N-(6-methylpyridin-2-yl)-4,5,7,8-tetrahydrobenzo-1H-oxepino[4,5-c]pyrazole-3-carboxamide; 1-methyl-N-(6-methylpyridin-2-yl)-1,4,5,6-tetrahydrobenzo[e]pyrazolo[4,3-c]azepine-3-carboxamide; 1-methyl-N-(6-methylpyridin-2-yl)-4,5,7,8-tetrahydrobenzo-1H-thiepino[4,5-c]pyrazole-3-carboxamide; 1-methyl-N-(6-methylpyridin-2-yl)-6-dioxo-4,5,7,8-tetrahydro-7,8-(2-pyridino)-1H-thiepino[4,5-c]pyrazole-3-carboxamide; 1-methyl-3-(1-(6-methylpyridin-2-yl)azetidin-3-yl)-4,5-dihydro-1H-pyrazolo[4,3-h]quinoline; 1-methyl-N-(6-methylpyridin-2-yl)-4,5-dihydro-1H-pyrazolo[4,3-h]quinolin-3-amine; 1-methyl-N-(1-(6-methylpyridin-2-yl)ethyl)-4,5-dihydro-1H-pyrazolo[4,3-h]quinolin-3-amine; 1-methyl-3-(1-(6-methylpyridin-2-yl)ethoxy)-4,5-dihydro-1H-pyrazolo[4,3-h]quinoline; 1-methyl-N-(6-methylpyridin-2-yl)-4,5-dihydro-1H-pyrazolo[3,4-f]isoquinoline-3-carboxamide; 5-hydroxy-1-methyl-N-(6-methylpyridin-2-yl)-4,5-dihydro-1H-benzo[g]indazole-3-carboxamide; 1-methyl-N-(6-methylpyridin-2-yl)-1,4,5,6,7,8-hexahydro-6-hydroxybenzocyclohepta[c]pyrazole-3-carboxamide; 1-methyl-N-(6-methylpyridin-2-yl)-1,4,5,6,7,8-hexahydro-6,6-difluorobenzocyclohepta[c]pyrazole-3-carboxamide; 5-hydroxy-1-methyl-N-(6-methylpyridin-2-yl)-4,5-dihydro-1H-pyrazolo[4,3-h]quinoline-3-carboxamide; 1-methyl-N-(6-methylpyridin-2-yl)-1,4,5,6,7,8-hexahydro-6-hydroxy-7,8-(2-pyridino)cyclohepta[c]pyrazole-3-carboxamide; 1-methyl-N-(6-methylpyridin-2-yl)-1,4,5,6,7,8-hexahydro-6,6-difluoro-7,8-(2-pyridino)cyclohepta[c]pyrazole-3-carboxamide; N-(3-chlorophenyl)-N-methyl-4H-chromeno[3,4-d]isoxazole-3-carboxamide; 1-methyl-N-(3-(trifluoromethyl)phenyl)-1,4-dihydrochromeno[4,3-c]pyrazole-3-carboxamide; N-(3-chlorophenyl)-N,1-dimethyl-1,4-dihydrochromeno[4,3-c]pyrazole-3-carboxamide; N-(2-chlorophenyl)-1-methyl-1,4-dihydrochromeno[4,3-c]pyrazole-3-carboxamide; N-(4-chlorophenyl)-1-methyl-1,4-dihydrochromeno[4,3-c]pyrazole-3-carboxamide; N-(4-chlorophenyl)-4,5-dihydro-1H-benzo[g]indazole-3-carboxamide; N-(pyridin-3-yl)-1,4-dihydrochromeno[4,3-c]pyrazole-3-carboxamide; N-(3-methoxyphenyl)-1,4-dihydrochromeno[4,3-c]pyrazole-3-carboxamide; N-(4-methoxyphenyl)-1,4-dihydrochromeno[4,3-c]pyrazole-3-carboxamide; N-(3-chlorophenyl)-1,8-dimethyl-1,4-dihydrothiochromeno[4,3-c]pyrazole-3-carboxamide; N-(4-fluorophenyl)-1-methyl-1,4-dihydrochromeno[4,3-c]pyrazole-3-carboxamide; N-(3-chloro-4-fluorophenyl)-1-methyl-1,4-dihydrochromeno[4,3-c]pyrazole-3-carboxamide; N-(5-chloro-2-methoxyphenyl)-1-methyl-1,4-dihydrochromeno[4,3-c]pyrazole-3-carboxamide; N-(3-methoxyphenyl)-1-methyl-1,4-dihydrochromeno[4,3-c]pyrazole-3-carboxamide; N-(5-chloropyridin-2-yl)-1-methyl-1,4-dihydrochromeno[4,3-c]pyrazole-3-carboxamide; N-(3-chlorophenyl)-1-methyl-1,4-dihydrothiochromeno[4,3-c]pyrazole-3-carboxamide; N-(3-chlorobenzyl)-1-methyl-1,4-dihydrochromeno[4,3-c]pyrazole-3-carboxamide; N-(3-chlorophenyl)-1-methyl-4,5-dihydro-1H-thieno[2,3-g]indazole-3-carboxamide; N-(3-chlorophenyl)-4H-thiochromeno[3,4-d]isoxazole-3-carboxamide; [1]Benzopyrano[4,3-c]pyrazole-3-carboxamide,1,4-dihydro-1-methyl-N-(6-methyl-2-pyridinyl)-; [1]Benzopyrano[4,3-c]pyrazole-3-carboxamide, 1,4-dihydro-1-methyl-N-2-pyridinyl-; [1]Benzopyrano[4,3-c]pyrazole-3-carboxamide, N-[3-(3-fluorophenyl)-5-isoxazolyl]-1,4-dihydro-1-methyl-; [1]Benzopyrano[4,3-c]pyrazole-3-carboxamide, N-[3-(2-ethoxyphenyl)-5-isoxazolyl]-1,4-dihydro-1-methyl-; [1]Benzopyrano[4,3-c]pyrazole-3-carboxamide, 1,4-dihydro-1-methyl-N-(5-methyl-2-pyridinyl)-; [1]Benzopyrano[4,3-c]pyrazole-3-carboxamide, N-[3-(4-ethoxyphenyl)-5-isoxazolyl]-1,4-dihydro-1-methyl-; [1]Benzopyrano[4,3-c]pyrazole-3-carboxamide, N-[3-(3-chlorophenyl)-5-isoxazolyl]-1,4-dihydro-1-methyl-; [1]Benzopyrano[4,3-c]pyrazole-3-carboxamide, 1,4-dihydro-1-methyl-N-3-pyridinyl-; 1-methyl-N-(5-methylisoxazol-3-yl)-1,4-dihydrochromeno[4,3-c]pyrazole-3-carboxamide; [1]Benzopyrano[4,3-c]pyrazole-3-carboxamide, N-(3,5-dimethylphenyl)-1,4-dihydro-1-methyl-; Benzoic acid, 4-[[(1,4-dihydro-1-methyl[1]benzopyrano[4,3-c]pyrazol-3-yl)carbonyl]amino]-, methyl ester; [1]Benzopyrano[4,3-c]pyrazole-3-carboxamide, N-[3-(3,4-diethoxyphenyl)-5-isoxazolyl]-1,4-dihydro-1-methyl-; [1]Benzopyrano[4,3-c]pyrazole-3-carboxamide, N-[4-[(4-ethyl-1-piperazinyl)methyl]phenyl]-1,4-dihydro-1-methyl-; [1]Benzopyrano[4,3-c]pyrazole-3-carboxamide, 1,4-dihydro-1-methyl-N-[4-[(4-methyl-1-piperidinyl)methyl]phenyl]-; [1]Benzopyrano[4,3-c]pyrazole-3-carboxamide, 1,4-dihydro-1-methyl-N-[4-(1-piperidinylsulfonyl)phenyl]-; [1]Benzopyrano[4,3-c]pyrazole-3-carboxamide, N-[4-[(diethylamino)sulfonyl]phenyl]-1,4-dihydro-1-methyl-; 1,3-Benzodioxole-5-carboxylic acid, 6-[[(1,4-dihydro-1-methyl[1]benzopyrano[4,3-c]pyrazol-3-yl)carbonyl]amino]-, methyl ester; [1]Benzopyrano[4,3-c]pyrazole-3-carboxamide, 1,4-dihydro-1-methyl-N-(4-methyl-2-oxo-2H-1-benzopyran-7-yl)-; 2-Thiophenecarboxylic acid, 3-[[(1,4-dihydro-1-methyl[1]benzopyrano[4,3-c]pyrazol-3-yl)carbonyl]amino]-, methyl ester; [1]Benzopyrano[4,3-c]pyrazole-3-carboxamide, N-(2,5-difluorophenyl)-1,4-dihydro-1-methyl-; [1]Benzopyrano[4,3-c]pyrazole-3-carboxamide, N-(3-acetylphenyl)-1,4-dihydro-1-methyl-; Benzoic acid, 4-[[(1,4-dihydro-1-methyl[1]benzopyrano[4,3-c]pyrazol-3-yl)carbonyl]amino]-, ethyl ester; [1]Benzopyrano[4,3-c]pyrazole-3-carboxamide, N-[4-(aminosulfonyl)phenyl]-1,4-dihydro-1-methyl-; [1]Benzopyrano[4,3-c]pyrazole-3-carboxamide, N-(4-chlorophenyl)-1,4-dihydro-1-methyl-; [1]Benzopyrano[4,3-c]pyrazole-3-carboxamide, N-(2-chlorophenyl)-1,4-dihydro-1-methyl-; [1]Benzopyrano[4,3-c]pyrazole-3-carboxamide, N-(2-cyanophenyl)-1,4-dihydro-1-methyl-; N-(2-carbamoylphenyl)-1-methyl-1,4-dihydrochromeno[4,3-c]pyrazole-3-carboxamide; [1]Benzopyrano[4,3-c]pyrazole-3-carboxamide, 1,4-dihydro-N-(4-methoxyphenyl)-1-methyl-; [1]Benzopyrano[4,3-c]pyrazole-3-carboxamide, N-(2-chloro-3-pyridinyl)-1,4-dihydro-1-methyl-; [1]Benzopyrano[4,3-c]pyrazole-3-carboxamide, N-(4-acetylphenyl)-1,4-dihydro-1-methyl-; 1,3-Benzenedicarboxylic acid, 5-[[(1,4-dihydro-1-methyl[1]benzopyrano[4,3-c]pyrazol-3-yl)carbonyl]amino]-, 1,3-dimethyl ester; [1]Benzopyrano[4,3-c]pyrazole-3-carboxamide, N-(2-ethylphenyl)-1,4-dihydro-1-methyl-; [1]Benzopyrano[4,3-c]pyrazole-3-carboxamide, 1,4-dihydro-1-methyl-N-(2-methylphenyl)-; [1]Benzopyrano[4,3-c]pyrazole-3-carboxamide, 1,4-dihydro-1-methyl-N-[2-(4-methyl-1-piperazinyl)phenyl]-; Benzoic acid, 4-[[(1,4-dihydro-1-methyl[1]benzopyrano[4,3-c]pyrazol-3-yl)carbonyl]amino]-, 2-(diethylamino)ethyl ester; [1]Benzopyrano[4,3-c]pyrazole-3-carboxamide, N-(5-chloro-2-pyridinyl)-1,4-dihydro-1-methyl-; [1]Benzopyrano[4,3-c]pyrazole-3-carboxamide, 1,4-dihydro-1-methyl-N-[3-(methylthio)phenyl]-; [1]Benzopyrano[4,3-c]pyrazole-3-carboxamide, N-(3-ethylphenyl)-1,4-dihydro-1-methyl-; [1]Benzopyrano[4,3-c]pyrazole-3-carboxamide, N-(9-ethyl-9H-carbazol-3-yl)-1,4-dihydro-1-methyl-; [1]Benzopyrano[4,3-c]pyrazole-3-carboxamide, 1,4-dihydro-1-methyl-N-(2-methyl-3-nitrophenyl)-; [1]Benzopyrano[4,3-c]pyrazole-3-carboxamide, N-(2-chloro-4-methylphenyl)-1,4-dihydro-1-methyl-; [1]Benzopyrano[4,3-c]pyrazole-3-carboxamide, N-(4-chloro-2-methoxy-5-methylphenyl)-1,4-dihydro-1-methyl-; [1]Benzopyrano[4,3-c]pyrazole-3-carboxamide, 1,4-dihydro-1-methyl-N-[4-(1-methylethyl)phenyl]-; [1]Benzopyrano[4,3-c]pyrazole-3-carboxamide, N-(4-ethylphenyl)-1,4-dihydro-1-methyl-; [1]Benzopyrano[4,3-c]pyrazole-3-carboxamide, 1,4-dihydro-N-(2-methoxy-5-methylphenyl)-1-methyl-; [1]Benzopyrano[4,3-c]pyrazole-3-carboxamide, N-(2-ethoxyphenyl)-1,4-dihydro-1-methyl-; Benzoic acid, 2-[[(1,4-dihydro-1-methyl[1]benzopyrano[4,3-c]pyrazol-3-yl)carbonyl]amino]-, ethyl ester; [1]Benzopyrano[4,3-c]pyrazole-3-carboxamide, 1,4-dihydro-N-(2-methoxy-5-nitrophenyl)-1-methyl-; [1]Benzopyrano[4,3-c]pyrazole-3-carboxamide, N-(2,5-dimethoxyphenyl)-1,4-dihydro-1-methyl-; [1]Benzopyrano[4,3-c]pyrazole-3-carboxamide, N-(4-ethoxyphenyl)-1,4-dihydro-1-methyl-; Benzoic acid, 2-[[(1,4-dihydro-1-methyl[1]benzopyrano[4,3-c]pyrazol-3-yl)carbonyl]amino]-, methyl ester; [1]Benzopyrano[4,3-c]pyrazole-3-carboxamide, N-(2,3-dihydro-1,4-benzodioxin-6-yl)-1,4-dihydro-1-methyl; [1]Benzopyrano[4,3-c]pyrazole-3-carboxamide, N-(4-bromo-2-methylphenyl)-1,4-dihydro-1-methyl-; [1]Benzopyrano[4,3-c]pyrazole-3-carboxamide, N-(5-chloro-2,4-dimethoxyphenyl)-1,4-dihydro-1-methyl-; [1]Benzopyrano[4,3-c]pyrazole-3-carboxamide, 1,4-dihydro-1-methyl-N-(3,4,5-trimethoxyphenyl)-; [1]Benzopyrano[4,3-c]pyrazole-3-carboxamide, 1,4-dihydro-N-(3-methoxyphenyl)-1-methyl-; [1]Benzopyrano[4,3-c]pyrazole-3-carboxamide, N-(2-fluorophenyl)-1,4-dihydro-1-methyl-; [1]Benzopyrano[4,3-c]pyrazole-3-carboxamide, N-(5-chloro-2-methoxyphenyl)-1,4-dihydro-1-methyl-; [1]Benzopyrano[4,3-c]pyrazole-3-carboxamide, N-(2,4-difluorophenyl)-1,4-dihydro-1-methyl-; [1]Benzopyrano[4,3-c]pyrazole-3-carboxamide, N-(2,4-dimethoxyphenyl)-1,4-dihydro-1-methyl-; [1]Benzopyrano[4,3-c]pyrazole-3-carboxamide, N-(3-chloro-4-methoxyphenyl)-1,4-dihydro-1-methyl-; [1]Benzopyrano[4,3-c]pyrazole-3-carboxamide, N-(3-chloro-4-fluorophenyl)-1,4-dihydro-1-methyl-; [1]Benzopyrano[4,3-c]pyrazole-3-carboxamide, N-(2-bromo-4-methylphenyl)-1,4-dihydro-1-methyl-; [1]Benzopyrano[4,3-c]pyrazole-3-carboxamide, 1,4-dihydro-1-methyl-N-[3-(trifluoromethyl)phenyl]-; [1]Benzopyrano[4,3-c]pyrazole-3-carboxamide, N-(3-chlorophenyl)-1,4-dihydro-1-methyl-; [1]Benzopyrano[4,3-c]pyrazole-3-carboxamide, N-(4-fluorophenyl)-1,4-dihydro-1-methyl-; [1]Benzopyrano[4,3-c]pyrazole-3-carboxamide, 1,4-dihydro-N-(2-methoxyphenyl)-1-methyl-; and pharmaceutically acceptable salts or N-oxides thereof, and, in some embodiments, a pharmaceutically acceptable carrier.

Also contemplated herein are compounds selected from the group consisting of: N-(3-chlorophenyl)-1-methyl-4,5-dihydro-1H-benzo[g]indazole-3-carboxamide; 8-chloro-N-(3-chlorophenyl)-1-methyl-1,4-dihydrochromeno[4,3-c]pyrazole-3-carboxamide; N-(3-chlorophenyl)-8-fluoro-2-methyl-2,4-dihydrochromeno[4,3-c]pyrazole-3-carboxamide; N-(4-chloropyridin-2-yl)-1-methyl-1,4-dihydrochromeno[4,3-c]pyrazole-3-carboxamide; N-(3-chlorophenyl)-8-fluoro-1-methyl-1,4-dihydrochromeno[4,3-c]pyrazole-3-carboxamide; N-(3-chlorophenyl)-1,4,4-trimethyl-1,4-dihydrochromeno[4,3-c]pyrazole-3-carboxamide; 7-fluoro-1-methyl-N-(6-methylpyridin-2-yl)-1,4-dihydrochromeno[4,3-c]pyrazole-3-carboxamide; 3-chloro-N-((1-methyl-1,4-dihydrochromeno[4,3-c]pyrazol-3-yl)methyl)aniline; 1-methyl-N-(6-methylpyridin-2-yl)-4,5-dihydro-1H-pyrazolo[4,3-h]quinoline-3-carboxamide; N-(6-methylpyridin-2-yl)-1,4-dihydrochromeno[4,3-c]pyrazole-3-carboxamide; N-(4-cyanopyridin-2-yl)-1-methyl-1,4-dihydrochromeno[4,3-c]pyrazole-3-carboxamide; N-(2,3-dihydrobenzo[b][1,4]dioxin-6-yl)-1-methyl-1,4-dihydrochromeno[4,3-c]pyrazole-3-carboxamide; (2H-benzo[b][1,4]oxazin-4(3H)-yl)(1-methyl-1,4-dihydrochromeno[4,3-c]pyrazol-3-yl)methanone; 5-acetyl-N-(3-chlorophenyl)-1-methyl-4,5-dihydro-1H-pyrazolo[4,3-c]quinoline-3-carboxamide; N-(3-chlorophenyl)-1-methyl-1H-pyrazolo[4,3-c]quinoline-3-carboxamide; N-(3-chlorophenyl)-1-methyl-4,5-dihydro-1H-pyrazolo[4,3-c]quinoline-3-carboxamide; 5-bromo-N-ethyl-N-((1-methyl-1,4-dihydrochromeno[4,3-c]pyrazol-3-yl)methyl)furan-2-carboxamide; 1-isopropyl-N-(6-methylpyridin-2-yl)-4,5-dihydro-1H-pyrazolo[4,3-h]quinoline-3-carboxamide; 1-ethyl-N-(6-methylpyridin-2-yl)-4,5-dihydro-1H-pyrazolo[4,3-h]quinoline-3-carboxamide; 1-methyl-N-(6-methylpyridin-2-yl)-1,4,5,6,7,8-hexahydro-7,8-(2-pyridino)cyclohepta[c]pyrazole-3-carboxamide; 1,8-dimethyl-N-(6-methylpyridin-2-yl)-4,5-dihydro-1H-pyrazolo[4,3-h]quinoline-3-carboxamide; 1-methyl-N-(6-methylpyridin-2-yl)-4,5-dihydro-1H-pyrazolo[4,3-h]isoquinoline-3-carboxamide; 1-methyl-N-(6-methylpyridin-2-yl)-4,5-dihydro-1H-pyrazolo[3,4-f]quinoline-3-carboxamide; 1,5-dimethyl-N-(6-methylpyridin-2-yl)-1,4,5,6-tetrahydropyrazolo[4,3-c]pyrido[2,3-e]azepine-3-carboxamide; 1,6-dimethyl-N-(6-methylpyridin-2-yl)-1,4,5,6-tetrahydropyrazolo[3,4-d]pyrido[3,2-b]azepine-3-carboxamide; 1-methyl-N-(6-methylpyridin-2-yl)-6-oxo-1,4,5,6-tetrahydrobenzo[e]pyrazolo[4,3-c]azepine-3-carboxamide; 1-methyl-N-(6-methylpyridin-2-yl)-5-oxo-1,4,5,6-tetrahydrobenzo[b]pyrazolo[3,4-d]azepine-3-carboxamide; 5,5-difluoro-1-methyl-N-(6-methylpyridin-2-yl)-4,5-dihydro-1H-pyrazolo[4,3-h]quinoline-3-carboxamide; 5,5-difluoro-1-methyl-N-(6-methylpyridin-2-yl)-4,5-dihydro-1H-benzo[g]indazole-3-carboxamide; 1,6-dimethyl-N-(6-methylpyridin-2-yl)-1,4,5,6-tetrahydrobenzo[b]pyrazolo[3,4-d]azepine-3-carboxamide; 1,5-dimethyl-N-(6-methylpyridin-2-yl)-4,5-dihydro-1H-pyrazolo[4,3-c]quinoline-3-carboxamide; 8-fluoro-1,5-dimethyl-N-(6-methylpyridin-2-yl)-4,5-dihydro-1H-pyrazolo[4,3-c]quinoline-3-carboxamide; 1,5-dimethyl-N-(6-methylpyridin-2-yl)-1,4,5,6-tetrahydrobenzo[e]pyrazolo[4,3-c]azepine-3-carboxamide; 1-methyl-N-(6-methylpyridin-2-yl)-5-oxo-4,5-dihydro-1H-pyrazolo[4,3-h]quinoline-3-carboxamide; 1-methyl-N-(6-methylpyridin-2-yl)-4H-benzo[b]imidazo[1,5-d][1,4]oxazine-3-carboxamide; 1-methyl-N-(6-methylpyridin-2-yl)-5-oxo-4,5-dihydro-1H-pyrazolo[4,3-h]quinoline-3-carboxamide; 1-methyl-N-(6-methylpyridin-2-yl)-4H-benzo[b]imidazo[1,5-d][1,4]oxazine-3-carboxamide; 1-methyl-N-(6-methylpyridin-2-yl)-5-oxo-4,5-dihydro-1H-pyrazolo[4,3-c]isoquinoline-3-carboxamide; 1,5-dimethyl-N-(6-methylpyridin-2-yl)-4,5-dihydro-1H-pyrazolo[4,3-c][1,5]naphthyridine-3-carboxamide; 9-fluoro-1-methyl-N-(6-methylpyridin-2-yl)-6-oxo-1,4,5,6-tetrahydrobenzo[e]pyrazolo[4,3-c]azepine-3-carboxamide; 1,5,5-trimethyl-N-(6-methylpyridin-2-yl)-4,5-dihydro-1H-pyrazolo[4,3-h]quinoline-3-carboxamide; 1-methyl-N-(6-(trifluoromethyl)pyridin-2-yl)-4,5-dihydro-1H-pyrazolo[4,3-h]quinoline-3-carboxamide; 1-methyl-N-(6-methylpyridin-2-yl)-4,5-dihydro-1H-pyrazolo[3,4-f]quinoxaline-3-carboxamide; 9-fluoro-1-methyl-N-(6-methylpyridin-2-yl)-4,5-dihydro-1H-pyrazolo[3,4-f]quinoline-3-carboxamide; 8-fluoro-1-methyl-N-(6-methylpyridin-2-yl)-4,5-dihydro-1H-pyrazolo[3,4-f]quinoline-3-carboxamide; 7-fluoro-1-methyl-N-(6-methylpyridin-2-yl)-4,5-dihydro-1H-pyrazolo[4,3-h]quinoline-3-carboxamide; 6-fluoro-1-methyl-N-(6-methylpyridin-2-yl)-4,5-dihydro-1H-pyrazolo[4,3-h]quinoline-3-carboxamide; N-(6-methylpyridin-2-yl)-4H-benzo[b]imidazo[1,5-d][1,4]oxazine-3-carboxamide; N-(6-cyclopropylpyridin-2-yl)-1-methyl-4,5-dihydro-1H-pyrazolo[4,3-h]quinoline-3-carboxamide; 1-methyl-N-(1-methyl-5-phenyl-1H-pyrrol-2-yl)-4,5-dihydro-1H-pyrazolo[4,3-h]quinoline-3-carboxamide; 6-methyl-N-(2-(1-methyl-4,5-dihydro-1H-pyrazolo[4,3-h]quinolin-3-yl)propan-2-yl)pyridin-2-amine; 3-(2-(3-chlorophenoxy)propan-2-yl)-1-methyl-4,5-dihydro-1H-pyrazolo[4,3-h]quinoline; 6-methyl-N-(1-methyl-4,5-dihydro-1H-pyrazolo[4,3-h]quinolin-3-yl)picolinamide; 3-(1-(3-chlorophenoxy)ethyl)-1-methyl-4,5-dihydro-1H-pyrazolo[4,3-h]quinoline; 6-cyclopropyl-N-(1-(1-methyl-4,5-dihydro-1H-pyrazolo[4,3-h]quinolin-3-yl)ethyl)pyridin-2-amine; 1-methyl-N-(6-methylpyridin-2-yl)-1,4,5,6-tetrahydro-6,7-(2-pyridino)cyclopenta[c]pyrazole-3-carboxamide; 1-methyl-N-(2-methylpyrimidin-4-yl)-4,5-dihydro-1H-pyrazolo[4,3-h]quinoline-3-carboxamide; 1-methyl-N-(6-methylpyrazin-2-yl)-4,5-dihydro-1H-pyrazolo[4,3-h]quinoline-3-carboxamide; 9-fluoro-1-methyl-N-(6-methylpyridin-2-yl)-5-oxo-1,4,5,6-tetrahydrobenzo[b]pyrazolo[3,4-d]azepine-3-carboxamide; 1-methyl-N-(6-methylpyridin-2-yl)-4,5,7,8-tetrahydrobenzo-1H-oxepino[4,5-c]pyrazole-3-carboxamide; 1-methyl-N-(6-methylpyridin-2-yl)-1,4,5,6-tetrahydrobenzo[e]pyrazolo[4,3-c]azepine-3-carboxamide; 1-methyl-N-(6-methylpyridin-2-yl)-4,5,7,8-tetrahydrobenzo-1H-thiepino[4,5-c]pyrazole-3-carboxamide; 1-methyl-N-(6-methylpyridin-2-yl)-6-dioxo-4,5,7,8-tetrahydro-7,8-(2-pyridino)-1H-thiepino[4,5-c]pyrazole-3-carboxamide; 1-methyl-3-(1-(6-methylpyridin-2-yl)azetidin-3-yl)-4,5-dihydro-1H-pyrazolo[4,3-h]quinoline; 1-methyl-N-(6-methylpyridin-2-yl)-4,5-dihydro-1H-pyrazolo[4,3-h]quinolin-3-amine; 1-methyl-N-(1-(6-methylpyridin-2-yl)ethyl)-4,5-dihydro-1H-pyrazolo[4,3-h]quinolin-3-amine; 1-methyl-3-(1-(6-methylpyridin-2-yl)ethoxy)-4,5-dihydro-1H-pyrazolo[4,3-h]quinoline; 1-methyl-N-(6-methylpyridin-2-yl)-4,5-dihydro-1H-pyrazolo[3,4-f]isoquinoline-3-carboxamide; 5-hydroxy-1-methyl-N-(6-methylpyridin-2-yl)-4,5-dihydro-1H-benzo[g]indazole-3-carboxamide; 1-methyl-N-(6-methylpyridin-2-yl)-1,4,5,6,7,8-hexahydro-6-hydroxybenzocyclohepta[c]pyrazole-3-carboxamide; 1-methyl-N-(6-methylpyridin-2-yl)-1,4,5,6,7,8-hexahydro-6,6-difluorobenzocyclohepta[c]pyrazole-3-carboxamide; 5-hydroxy-1-methyl-N-(6-methylpyridin-2-yl)-4,5-dihydro-1H-pyrazolo[4,3-h]quinoline-3-carboxamide; 1-methyl-N-(6-methylpyridin-2-yl)-1,4,5,6,7,8-hexahydro-6-hydroxy-7,8-(2-pyridino)cyclohepta[c]pyrazole-3-carboxamide; 1-methyl-N-(6-methylpyridin-2-yl)-1,4,5,6,7,8-hexahydro-6,6-difluoro-7,8-(2-pyridino)cyclohepta[c]pyrazole-3-carboxamide; and pharmaceutically acceptable salts or N-oxides thereof.

The present disclosure also provides pharmaceutical compositions comprising compounds as disclosed herein formulated together with one or more pharmaceutically acceptable carriers. These formulations include those suitable for oral, rectal, topical, buccal and parenteral (e.g., subcutaneous, intramuscular, intradermal, or intravenous) administration, although the most suitable form of administration in any given case will depend on the degree and severity of the condition being treated and on the nature of the particular compound being used.

The invention further provides methods of modulating activity of one or more metabotropic receptors-subtype 5 comprising exposing said receptor to a compound of the invention. In one embodiment, the modulator compound is a negative allosteric modulator.

The invention further provides methods of treating a disease associated with expression or activity of one or more metabotropic receptors-subtype 5 in a patient comprising administering to the patient a therapeutically effective amount of a compound of the invention.

In one embodiment of the invention provides a method of treating Alzheimer's disease, Fragile X syndrome, L-DOPA induced dyskinesia, depression, anxiety, migraine, pain, gastroesophageal reflux disease, and/or aiding smoking cessation comprising administering to a subject in need thereof a therapeutically effective amount of a compound of the invention. Other diseases and disorders that may be treated by administration of a therapeutically effective amount of a compound of the invention include functional bowel disorders, post-operative ileus, urinary tract disorders (incontinence, BPH, prostatitis, OAB), Parkinson's disease, panic disorder, phobia, posttraumatic stress disorder, generalized anxiety disorder, acute stress disorder, depression, bipolar disorder, attention-deficit/hyperactivity disorder, cognitive impairment, Alzheimer's disease, dementia, delirium tremens, ischemia, and neuropathic pain.

Compounds of the invention may also be effective in the treatment of abuse or addition to alcohol, nicotine, cocaine, amphetamine, benzodiazepine, and opiates. Compounds of the invention may also be useful in treating or preventing substance tolerance or dependence to bulimia nervosa, anorexia nervosa, gambling dependence, smoking dependence, sex dependence, substance withdrawal, and obsessive compulsive disorders.

The compounds of the invention may be administered to patients (animals and humans) in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors which those skilled in the art will recognize, with the appropriate dosage ultimately being at the discretion of the attendant physician. For treating clinical conditions and diseases noted above, the compound of this invention may be administered orally, topically, parenterally, by inhalation spray or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques.

Exemplary pharmaceutical compositions of this invention may be used in the form of a pharmaceutical preparation, for example, in solid, semisolid or liquid form, which contains one or more of the compound of the invention, as an active ingredient, in admixture with an organic or inorganic carrier or excipient suitable for external, enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The carriers which can be used are water, glucose, lactose, gum acacia, gelatin, mannitol, starch paste, magnesium trisilicate, talc, corn starch, keratin, colloidal silica, potato starch, urea and other carriers suitable for use in manufacturing preparations, in solid, semisolid, or liquid form, and in addition auxiliary, stabilizing, thickening and coloring agents and perfumes may be used. The active object compound is included in the pharmaceutical composition in an amount sufficient to produce the desired effect upon the process or condition of the disease.

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

The liquid forms in which the compositions of the invention may be incorporated for administration orally or by injection include aqueous solution, suitably flavored syrups, aqueous or oil suspensions, and emulsions with acceptable oils such as cottonseed oil, sesame oil, coconut oil or peanut oil, or with a solubilizing or emulsifying agent suitable for intravenous use, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin. Compositions for inhalation or insufflation include solutions and suspensions in pharmaceutically acceptable, aqueous or organic solvents, or mixtures thereof, and powders.

Advantageously, the invention also provides kits for use by a consumer having, or at risk of having, a disease or condition associated with metabotropic receptors-subtype 5. Such kits include a suitable dosage form such as those described above and instructions describing the method of using such dosage form to mediate, reduce or prevent inflammation. The instructions would direct the consumer or medical personnel to administer the dosage form according to administration modes known to those skilled in the art. Such kits could advantageously be packaged and sold in single or multiple kit units. An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of a first compound can consist of one tablet or capsule while a daily dose of the second compound can consist of several tablets or capsules and vice versa. The memory aid should reflect this.

### EXAMPLES

The compounds of the present invention can be prepared in a number of ways well known to one skilled in the art of organic synthesis. More specifically, compounds of the invention may be prepared using the reactions and techniques described herein. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

Schemes 1-8 depict methods for making exemplary compounds of the invention as described below.

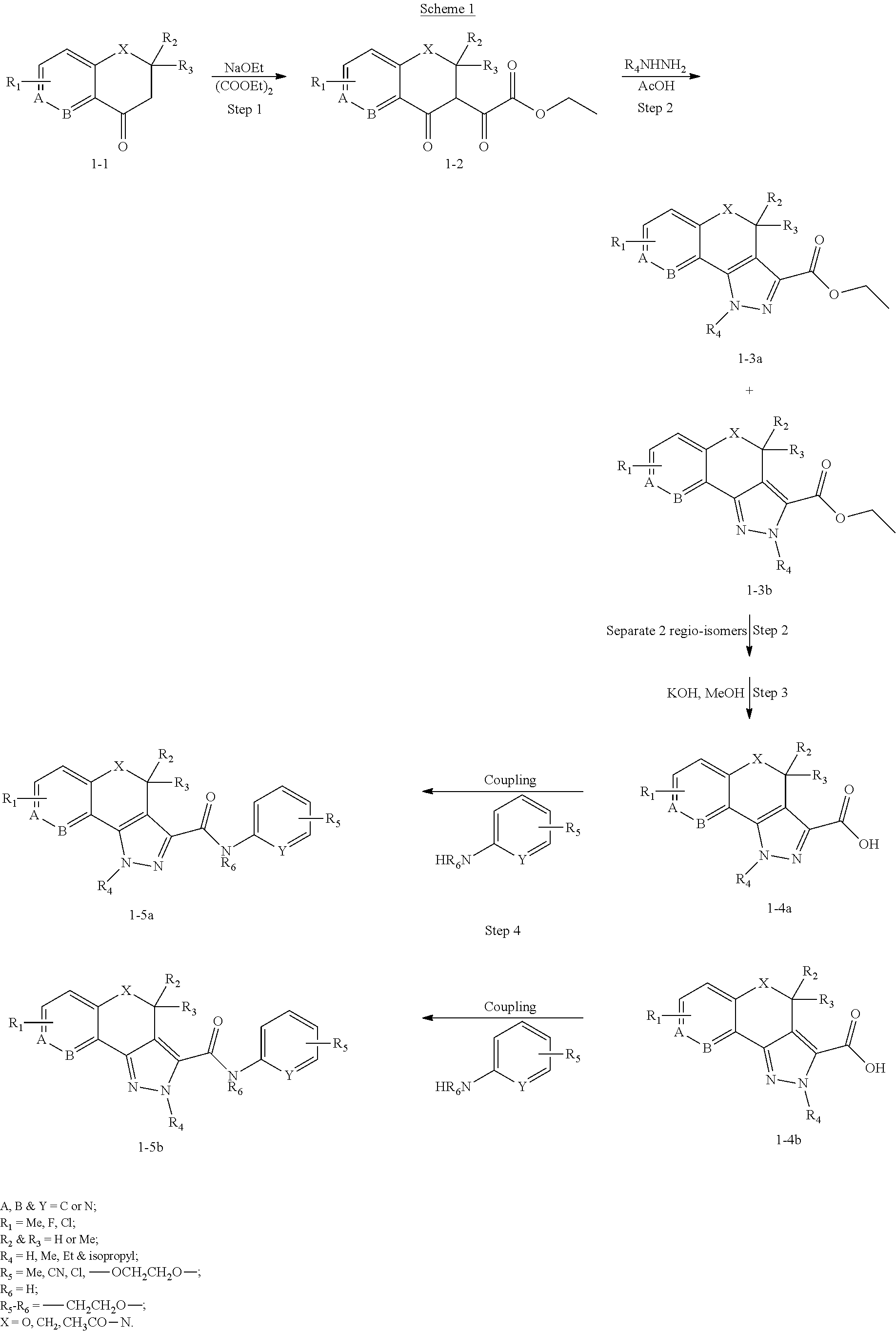

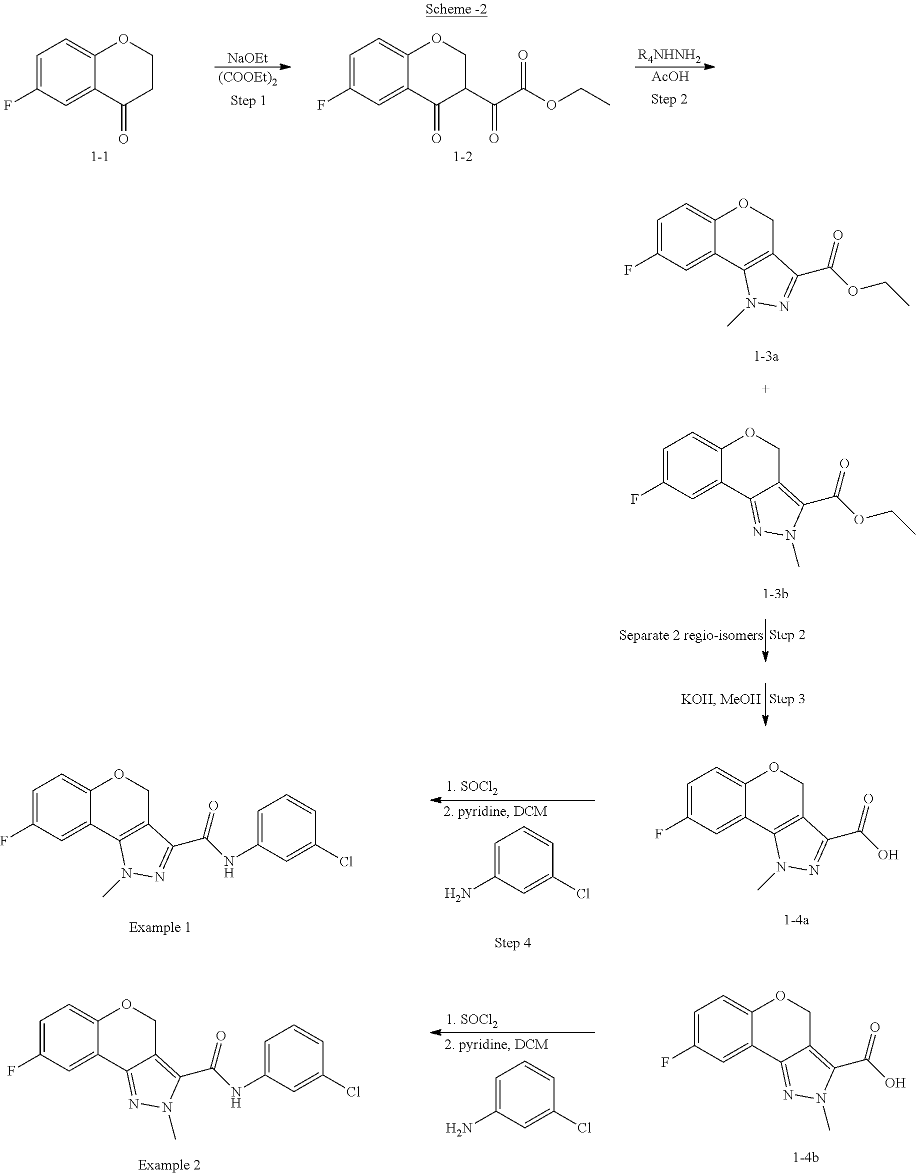

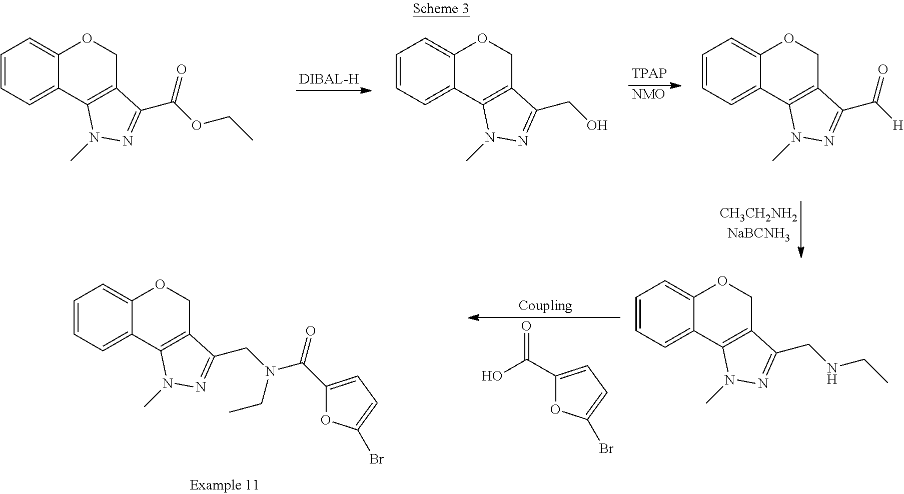

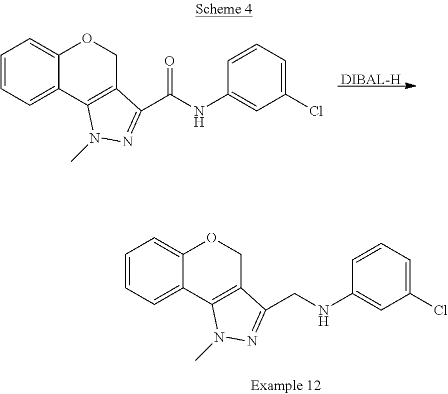

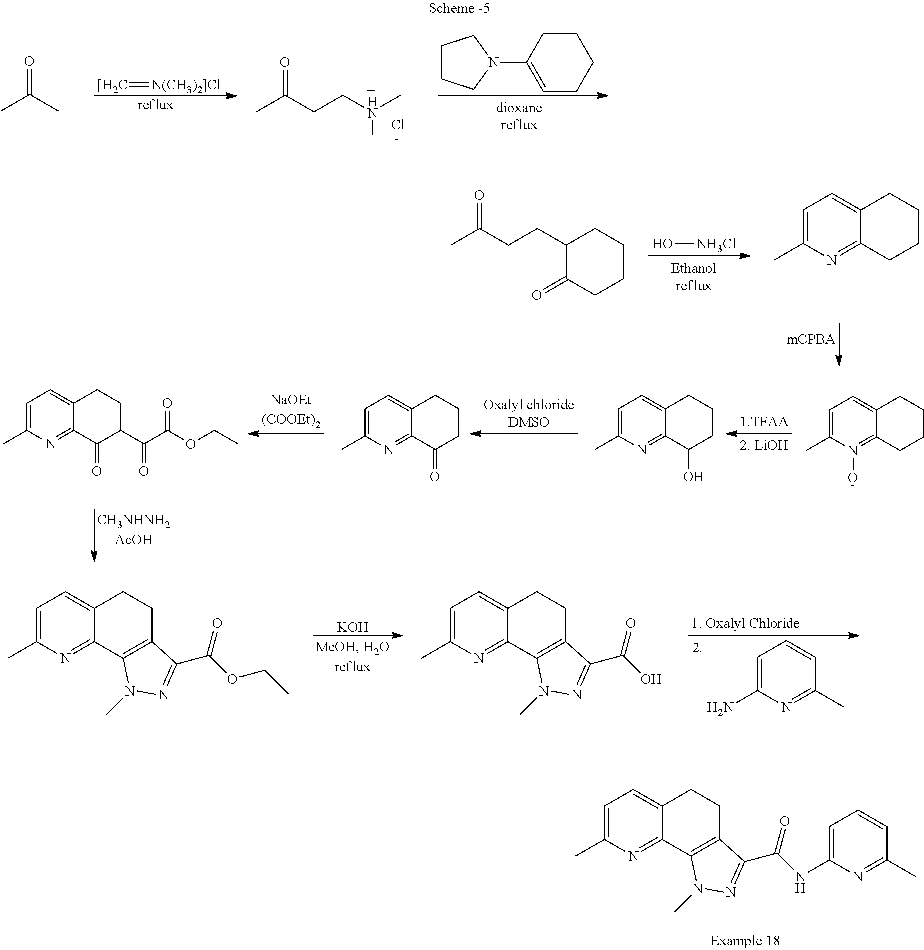

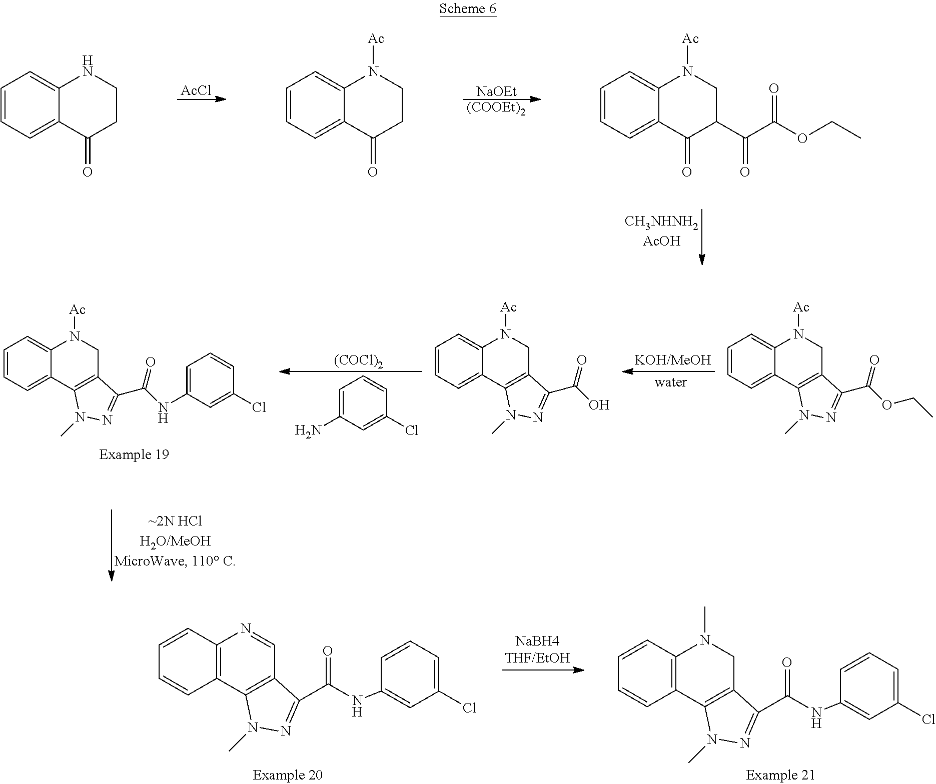

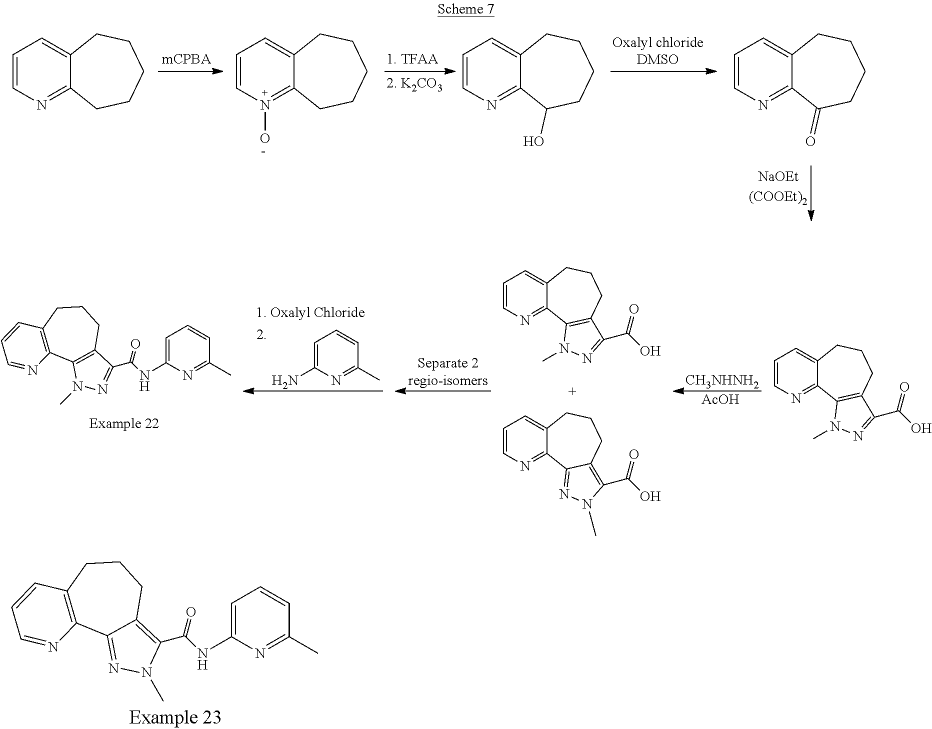

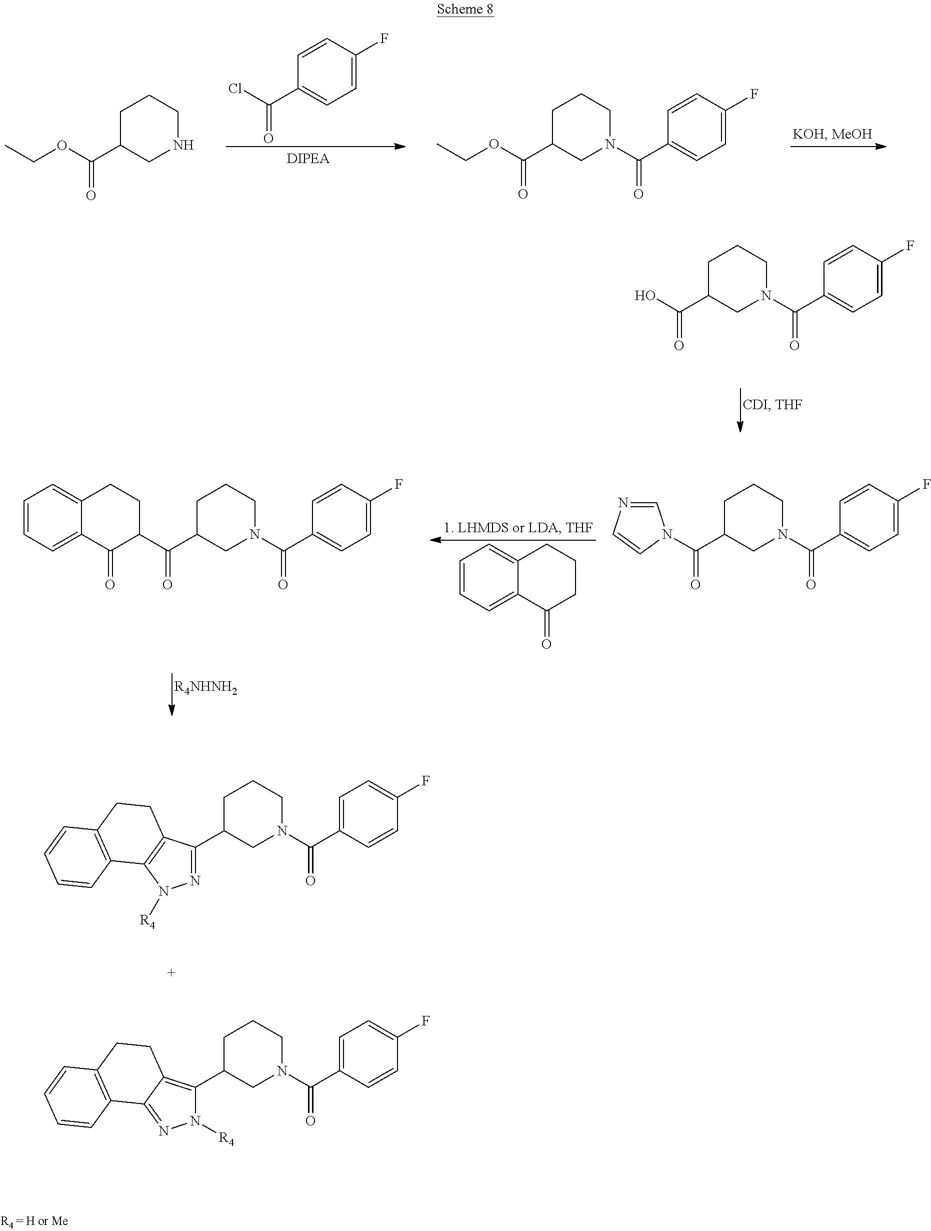

A: General Procedure for Synthesis of α,γ-diketoesters 1-2 (Step 1 of Scheme 1)

To a solution of appropriate chroman-4-one (or 3,4-dihydronaphthalen-1(2H)-one, or 2,3-dihydroquinolin-4(1H)-one) starting material (1.0 eq.) in ethanol was added a solution of sodium ethoxide (1.05 eq.). The mixture was stirred for 10 min at room temperature and then diethyl oxalate (1.0 eq.) was added. After the mixture was stirred overnight, 1N HCl solution was added to adjust the pH to 4-5. The product was extracted with ethyl acetate and dried over anhydrous sodium sulfate. The solvent was removed under reduced pressure to yield α,γ-diketoester 1-2, which was used in step 2 without further purification.

### B: General Procedure for Synthesis of Tricyclic Ester 1-3a and b (Step 2 of Scheme 1)

To a solution of α,γ-diketoester 1-2 (1.0 eq.) in acetic acid was added the appropriate hydrazine (1.1 eq.) at room temperature. The mixture was stirred overnight. The solvent was removed under reduced pressure. The reaction mixture was purified by silica chromatography on an ISCO system (EtOAc/hexanes) to collect 2 regio-isomers 1-3a and 1-3b.

### C: General Procedure for Synthesis of Tricyclic Acid (Step 3 of Scheme 1)

To a solution of α,γ-tricyclic ethyl ester 1-3a or 1-3b (1.0 eq.) in methanol was added a 1M solution of potassium hydroxide in methanol (6 eq.) and water. The resulting mixture was heated under reflux overnight. The mixture was allowed to cool to room temperature and then acidified with 1N HCl to pH 4-5. The product was extracted with ethyl acetate and dried over anhydrous sodium sulfate. The solvent was removed to yield tricyclic acid 1-4a or 1-4b.

### D: General Procedure for Synthesis of Tricyclic Carboxamides (Step 4 of Scheme 1)

To a solution of tricyclic acid 1-4a or 1-4b (1.0 eq.) and pyridine (3 eq.) in dichloromethane was added oxalyl chloride (1.1 eq.) at room temperature. The mixture was stirred for 2 h and then the appropriate aniline (1.2 eq.) was added. The mixture was stirred overnight. After the solvent was removed under reduced pressure, the reaction mixture was purified by silica chromatography on an ISCO system (EtOAc/hexanes) to yield tricyclic carboxamides; 1-5a or 1-5b.

### Example 1

### N-(3-Chlorophenol)-8-fluoro-1-methyl-1,4-dihydrochromeno[4,3-c[pyrazole-3-carboxamide

### Ethyl 2-(6-fluoro-4-oxochroman-3-yl)-2-oxoacetate

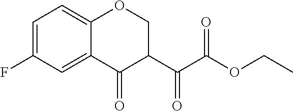

The title compound was prepared according to general procedure A described in Scheme 2. The title compound was obtained as a yellow solid (1.76 g, 6.62 mmol, 66.2% yield) by using 6-fluorochroman-4-one (1.66 g, 10.0 mmol), sodium ethoxide (21 wt % in ethanol, 7.46 mL, 20.0 mmol), diethyl oxalate (1.36 ml, 10.0 mmol) in ethanol (100 mL).

### Ethyl 8-fluoro-1-methyl-1,4-dihydrochromeno[4,3-c]pyrazole-3-carboxylate

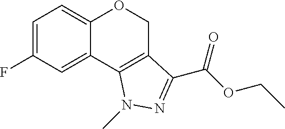

The title compound was prepared according to general procedure B described in Scheme 2. A mixture of ethyl 2-(6-fluoro-4-oxochroman-3-yl)-2-oxoacetate (1.6 g, 6.0 mmol) and methyl hydrazine (0.36 mL, 6.9 mmol) in ethanol (40 mL). was refluxed for 6 h. The solvent was removed under reduced pressure. The reaction mixture was purified by silica chromatography on an ISCO system (EtOAc/hexanes) to collect 2 regio-isomers. The title compound was obtained as a white solid (421.5 mg, 1.53 mmol, 25.5% yield) that was shown at retention time 4.6 min in LC-MS analysis (Kromasil 100-3.5 C4 column, 50×4.6 mm, 3.5 μm. Mobile phase A was 0.05% TFA in water; mobile phase B was 0.05% TFA in acetonitrile. The gradient was initiated at 2% B and ramp to 75% B in 7 min, followed by a wash to 95% B within 0.5 min and re-equilibration back to 2% from 8.6 min. The total analyzed time was 10 min. Flow rate was 0.8 mL/min). MS (ESI) m/z: Calculated for C₁₄H₁₃FN₂O₃: 276.1; found: 277.0 (M+H)⁺.

### 8-Fluoro-1-methyl-1,4-dihydrochromeno[4,3-c]pyrazole-3-carboxylic acid

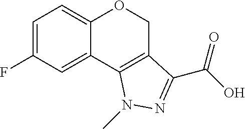

The title compound was prepared according to general procedure C described in Scheme 2. The title compound was obtained as a white solid (238 mg, 0.96 mmol, 81% yield) by using ethyl 8-fluoro-1-methyl-1,4-dihydrochromeno[4,3-c]pyrazole-3-carboxylate (330 mg, 1.19 mmol), 1N potassium hydroxide in methanol (7.2 mL, 7.2 mmol), methanol (60 ml) and water (1 mL). MS (ESI) m/z: Calculated for C₁₂H₉FN₂O₃: 248.1; found: 249.0 (M+H)⁺.

### N-(3-Chlorophenyl)-8-fluoro-1-methyl-1,4-dihydrochromeno[4,3-c]pyrazole-3-carboxamide

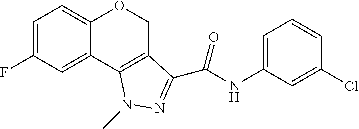

The title compound was prepared according to general procedure D described in Scheme 2. The title compound was obtained as a white solid with puritiy greater than 95% (51.7 mg, 0.145 mmol, 15% yield) by using 8-fluoro-1-methyl-1,4-dihydrochromeno[4,3-c]pyrazole-3-carboxylic acid (238 mg, 0.96 mmol), oxalyl chloride (0.53 mL, 1.05 mmol), pyridine (0.23 ml, 2.9 mmol), 3-chloroaniline (147 mg, 1.15 mmol) and dichloromethane (20 mL). ¹H NMR (282 MHz, CDCl₃): δ 8.61 (s, 1H), 7.81 (t, 1H), 7.50(m, 1H), 7.27 (t, 1H), 7.21 (dd, 1H), 7.10 (m, 1H), 6.98 (m, 2H), 5.54 (s, 2H), 4.18(s, 3H); ¹⁹F NMR (376 MHz, CDCl₃): δ −120.8; MS (ESI) m/z: Calculated for C₁₈H₁₃ClFN₃O₂: 357.1; found: 358.0 (M+H)⁺.

### Example 2

### N-(3-Chlorophenyl)-8-fluoro-2-methyl-2,4-dihydrochromeno[4,3-c]pyrazole-3-carboxamide

### Ethyl 8-fluoro-2-methyl-2,4-dihydrochromeno[4,3-c]pyrazole-3-carboxylate

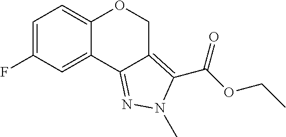

The title compound was prepared according to general procedure B described in Scheme 2. A mixture of ethyl 2-(6-fluoro-4-oxochroman-3-yl)-2-oxoacetate (1.6 g, 6.0 mmol) and methyl hydrazine (0.36 mL, 6.9 mmol) in ethanol (40 mL). was refluxed for 6 h. The solvent was removed under reduced pressure. The reaction mixture was purified by silica chromatography on an ISCO system (EtOAc/hexanes) to collect 2 regio-isomers. The title compound was obtained as a white solid (597.8 mg, 2.16 mmol, 36.0% yield) that was shown at the retention time 5.2 min in LC-MS analysis (Kromasil 100-3.5 C4 column, 50×4.6 mm, 3.5 μm. Mobile phase A was 0.05% TFA in water; mobile phase B was 0.05% TFA in acetonitrile. The gradient was initiated at 2% B and ramp to 75% B in 7 min, followed by a wash to 95% B within 0.5 min and re-equilibration back to 2% from 8.6 min. The total analyzed time was 10 min. Flow rate was 0.8 mL/min). MS (ESI) m/z: Calculated for C₁₄H₁₃FN₂O₃: 276.1; found: 277.0 (M+H)⁺.

### 8-Fluoro-2-methyl-2,4-dihydrochromeno[4,3-c]pyrazole-3-carboxylic acid

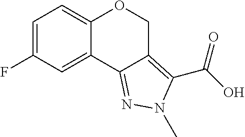

The title compound was prepared according to general procedure C described in Scheme 2. The title compound was obtained as a white solid (335 mg, 1.35 mmol, 85% yield) by using ethyl 8-fluoro-2-methyl-2,4-dihydrochromeno[4,3-c]pyrazole-3-carboxylate (440 mg, 1.59 mmol), 1N potassium hydroxide in methanol (9.6 mL, 9.6 mmol), methanol (80 ml) and water (2 mL). MS (ESI) m/z: Calculated for C₁₂H₉FN₂O₃: 248.1; found: 249.0 (M+H)⁺.

### N-(3-Chlorophenyl)-8-fluoro-2-methyl-2,4-dihydrochromeno[4,3-c]pyrazole-3-carboxamide

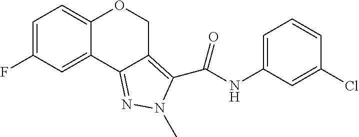

The title compound was prepared according to general procedure D described in Scheme 2. The title compound was obtained as a white solid with puritiy greater than 95% (38.6 mg, 0.108 mmol, 17% yield) by using 8-fluoro-2-methyl-2,4-dihydrochromeno[4,3-c]pyrazole-3-carboxylic acid (160 mg, 0.65 mmol), oxalyl chloride (0.36 mL, 0.71 mmol), pyridine (0.16 ml, 1.9 mmol), 3-chloroaniline (98 mg, 0.77 mmol) and dichloromethane (20 mL). ¹H NMR (300 MHz, CDCl₃): δ 7.69 (t, 1H), 7.48-7.42 (m, 1H), 7.41-7.36 (m, 1H), 7.32 (t, 1H), 7.22-7.15 (m, 2H), 6.94-6.90 (m, 2H), 5.39 (s, 2H), 4.18 (s, 3H); ¹⁹F NMR (376 MHz, CDCl₃): δ 121.1; MS (ESI) m/z: Calculated for C₁₈H₁₃ClFN₃O₂: 357.1; found: 358.0 (M+H)⁺.

### Example 3

### N-(4-Chloropyridin-2-yl)-1-methyl-1,4-dihydrochromeno[4,3-c[pyrazole-3-carboxamide

### Ethyl 2-oxo-2-(4-oxochroman-3-yl)acetate

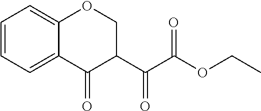

The title compound was prepared according to general procedure A described in Scheme 1. The title compound was obtained as a crude product by using 4-chromanone (5.0 g, 33.74 mmol), sodium ethoxide (21 wt % in ethanol, 15.1 mL, 40.5 mmol), diethyl oxalate (7.77 ml, 33.74 mmol) in ethanol (40 mL). MS (ESI) m/z: Calculated for C₁₃H₁₂O₅: 248.1; found: 249.0 (M+H)⁺.

### Ethyl 1-methyl-1,4-dihydrochromeno[4,3-c]pyrazole-3-carboxylate

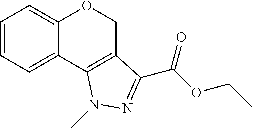

The title compound was prepared according to general procedure B described in Scheme 1. The title compound was obtained as a pale-yellow solid (1.1 g, 4.3 mmol, 53% yield) by using ethyl 2-oxo-2-(4-oxochroman-3-yl)acetate (2.0 g, 8.06 mmol), methyl hydrazine (0.47 ml, 8.86 mmol) and acetic acid (13 ml). The title compound was shown at the retention time 4.4 min in LC-MS analysis (Kromasil 100-3.5 C4 column, 50×4.6 mm, 3.5 μm. Mobile phase A was 0.05% TFA in water; mobile phase B was 0.05% TFA in acetonitrile. The gradient was initiated at 2% B and ramp to 75% B in 7 min, followed by a wash to 95% B within 0.5 min and re-equilibration back to 2% from 8.6 min. The total analyzed time was 10 min. Flow rate was 0.8 mL/min). MS (ESI) m/z: Calculated for C₁₄H₁₄N₂O₃: 258.1; found: 259.0 (M+H)⁺.

### 1-Methyl-1,4-dihydrochromeno[4,3-c]pyrazole-3-carboxylic acid

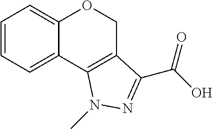

The title compound was prepared according to general procedure C described in Scheme 1. The title compound was obtained as a white solid (161 mg, 0.70 mmol, 99% yield) by using ethyl 1-methyl-1,4-dihydrochromeno[4,3-c]pyrazole-3-carboxyl ate (183 mg, 0.71 mmol), 1N potassium hydroxide in methanol (4.2 mL, 4.2 mmol), methanol (25 ml) and water (1 mL). MS (ESI) m/z: Calculated for C₁₂H₁₀N₂O₃: 230.1; found: 231.0 (M+H)⁺.

### N-(4-Chloropyridin-2-yl)-1-methyl-1,4-dihydrochromeno[4,3-c]pyrazole-3-carboxamide

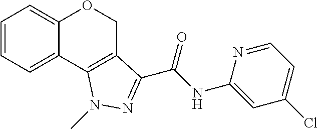

The title compound was prepared according to general procedure D described in Scheme 1. The title compound was obtained as a white solid with puritiy greater than 95% (25.4 mg, 0.07 mmol, 14% yield) by using 1-methyl-1,4-dihydrochromeno[4,3-c]pyrazole-3-carboxylic acid (119 mg, 0.52 mmol), oxalyl chloride (0.28 mL, 0.57 mmol), pyridine (0.13 ml, 1.6 mmol), 4-chloropyridin-2-amine (79.8 mg, 0.62 mmol) and dichloromethane (20 mL). ¹H NMR (400 MHz, CDCl₃): δ 9.32 (s, 1H), 8.43 (d, 1H), 8.23 (t, 1H), 7.53 (m, 1H), 7.25 (m, 1H), 7.06 (m, 3H), 5.59 (d, 2H), 4.20(s, 3H); MS (ESI) m/z: Calculated for C₁₇H₁₃ClN₄O₂: 340.1; found: 341.0 (M+H)⁺.

### Example 4

### N-(3-Chlorophenol)-1,4,4-trimethyl-1,4-dihydrochromeno[4,3-c]pyrazole-3-carboxamide

### Ethyl 2-(2,2-dimethyl-4-oxochroman-3-yl)-2-oxoacetate

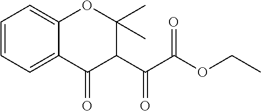

The title compound was prepared according to general procedure A described in Scheme 1. The title compound was obtained as a crude product by using 2,2-dimethylchroman-4-one (910 mg, 5.16 mmol), sodium ethoxide (21 wt % in ethanol, 2.02 mL, 5.42 mmol), diethyl oxalate (1.2 ml, 5.16 mmol) in ethanol (20 mL). MS (ESI) m/z: Calculated for C₁₅H₁₆O₅: 276.1; found: 277.0 (M+H)⁺.

### Ethyl 1,4,4-trimethyl-1,4-dihydrochromeno[4,3-c]pyrazole-3-carboxylate

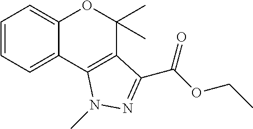

The title compound was prepared according to general procedure B described in Scheme 1. The title compound was obtained as a orange solid (473 mg, 1.65 mmol, 33% yield) by using ethyl 2-(2,2-dimethyl-4-oxochroman-3-yl)-2-oxoacetate (1.544 g, 5.0 mmol), methyl hydrazine (0.29 ml, 5.5 mmol) and acetic acid (10 ml). The title compound was shown at the retention time 5.0 min in LC-MS analysis (Kromasil 100-3.5 C4 column, 50×4.6 mm, 3.5 μm. Mobile phase A was 0.05% TFA in water; mobile phase B was 0.05% TFA in acetonitrile. The gradient was initiated at 2% B and ramp to 75% B in 7 min, followed by a wash to 95% B within 0.5 min and re-equilibration back to 2% from 8.6 min. The total analyzed time was 10 min. Flow rate was 0.8 mL/min). MS (ESI) m/z: Calculated for C₁₆H₁₈N₂O₃: 286.1; found: 287.2 (M+H)⁺.

### 1,4,4-Trimethyl-1,4-dihydrochromeno[4,3-c]pyrazole-3-carboxylic acid

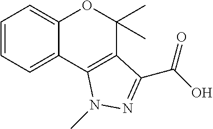

The title compound was prepared according to general procedure C described in Scheme 1. The title compound was obtained as a white solid (368 mg, 1.42 mmol, 91% yield) by using ethyl 1,4,4-trimethyl-1,4-dihydrochromeno[4,3-c]pyrazole-3-carboxylate (450 mg, 1.57 mmol), 1N potassium hydroxide in methanol (9.5 mL, 9.5 mmol), methanol (80 ml) and water (2 mL). MS (ESI) m/z: Calculated for C₁₄H₁₄N₂O₃: 258.1; found: 259.1 (M+H)⁺.

### N-(3-Chlorophenyl)-1,4,4-trimethyl-1,4-dihydrochromeno[4,3-c]pyrazole-3-carboxamide

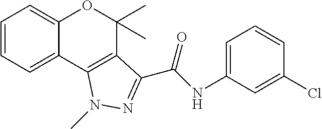

The title compound was prepared according to general procedure D described in Scheme 1. The title compound was obtained as a white solid with puritiy greater than 95% (69.7 mg, 0.33 mmol, 39% yield) by using 1,4,4-trimethyl-1,4-dihydrochromeno[4,3-c]pyrazole-3-carboxylic acid (214 mg, 0.83 mmol), oxalyl chloride (0.46 mL, 0.91 mmol), pyridine (0.20 ml, 2.5 mmol), 3-chloroanaline (127 mg, 0.99 mmol) and dichloromethane (20 mL). ¹H NMR (400 MHz, CDCl₃): δ 8.84 (s, 1H), 7.81 (m, 1H), 7.51 (m, 2H), 7.25 (m, 2H), 7.08 (m, 1H), 7.0 (m, 2H), 4.18 (d, 3H) 1.83 (d, 6H); MS (ESI) m/z: Calculated for C₂₀H₁₈ClN₃O₂: 367.1; found: 368.2 (M+H)⁺.

### Example 5

### N-(3-Chlorophenyl)-8-chloro-1-methyl-1,4-dihydrochromeno[4,3-c[pyrazole-3-carboxamide

### Ethyl 8-chloro-1-methyl-1,4-dihydrochromeno[4,3-c]pyrazole-3-carboxylate

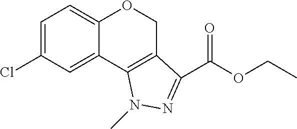

The title compound was prepared according to general procedure B described in Scheme 1. The title compound was obtained as a white solid (367 mg, 1.26 mmol, 32% yield) by using ethyl 2-(6-chloro-4-oxochroman-3-yl)-2-oxoacetate (1131 mg, 4 mmol), methyl hydrazine (242 uL, 4.6 mmol) and ethanol (28 ml). MS (ESI) m/z: Calculated for C₁₄H₁₃ClN₂O₃: 292.1; found: 293.0 (M+H)⁺.

### 8-Chloro-1-methyl-1,4-dihydrochromeno[4,3-c]pyrazole-3-carboxylic acid

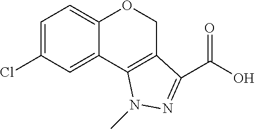

The title compound was prepared according to general procedure C described in Scheme 1. The title compound was obtained as a white solid (285 mg, 1.08 mmol, 86% yield) by using ethyl 8-chloro-1-methyl-1,4-dihydrochromeno[4,3-c]pyrazole-3-carboxylate (367 mg, 1.26 mmol), potassium hydroxide (141 mg, 2.51 mmol), methanol (15 ml). MS (ESI) m/z: Calculated for C₁₂H₉ClN₂O₃: 264.0; found: 265.0 (M+H)⁺.

### N-(3-Chlorophenyl)-8-chloro-1-methyl-1,4-dihydrochromeno[4,3-c]pyrazole-3-carboxamide

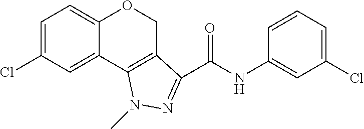

The title compound was prepared according to general procedure D described in Scheme 1. To a suspension of 8-chloro-1-methyl-1,4-dihydrochromeno[4,3-c]pyrazole-3-carboxylic acid (106 mg, 0.4 mmol) in methylene chloride (4 mL) was added a 2M (methylene chloride) solution of oxalyl chloride (1 mL, 5 mmol) followed by 1 drop of DMF. The resulting mixture was stirred at room temperature for 2 h. The mixture was concentrated under reduced pressure to provide a colorless solid. The solid was dissolved in methylene chloride (2 mL) and added dropwise to a solution of 3-chloroaniline (63 uL, 1.2 mM) and triethylamine (167 uL, 1.2 mmol) in methylene chloride (8 mL). The resulting mixture was stirred at room temperature for 18 h. The mixture was diluted with ethyl acetate and washed with 1N aq. HCl, water and brine. The organic fraction was dried (magnesium sulfate), filtered and concentrated. The residue was purified by recrystalization from ethyl acetate. Yield 48 mg of title compound (32% yield): ¹H NMR (300 MHz, CDCl₃): 8.60 (s, 1H), 7.82 (s, 1H), 7.48-6.96 (m, 5H), 5.58 (d, 2H), 4.20 (d, 3H). MS (ESI) m/z calculated for C₁₈H₁₃Cl₂N₃O₂: 373.0; found: 398.0 (M+Na⁺)⁺.

### Example 6

### 6-Methylpyridin-2-yl 7-fluoro-1-methyl-1,4-dihydrochromeno[4,2-c[pyrazole-3-carboxylate

### 7-Fluoro-1-methyl-1,4-dihydrochromeno[4,3-c]pyrazole-3-carboxylic acid

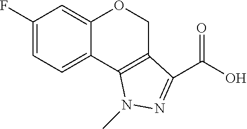

The title compound was prepared according to general procedure A, B and C described in Scheme 1. To a solution of ethanol solution of sodium ethoxide (21 wt %, 4.1 mL, 10.92 mmol) at 0° C. was added 7-fluorochroman-4-one (1.72 g, 10.4 mmol) as a solution in ethanol (20 mL), The mixture was stirred at 0° C. for 10 minutes and then diethyl oxalate (1.14 mL, 10.4 mL) was added dropwise. The ice-bath was removed and the solution was heated to 40° C. for 2.5 h. After cooling the mixture was concentrated to approx. 10 mL and diluted with water (30 mL). The pH of the solution was adjusted to 4.0 with 1N HCl and the mixture was extracted with ethyl acetate. The ethyl acetate fraction was dried (magnesium sulfate), filtered and concentrated. The product, ethyl 2-(7-fluoro-4-oxochroman-3-yl)-2-oxoacetate (2.78 g) was used as is without further purification.

To a mixture of 2-(7-fluoro-4-oxochroman-3-yl)-2-oxoacetate (2.78 g, 10.4 mmol) in acetic acid (15 mL) was added methyl hydrazine (602 uL, 11.44 mmol) dropwise. The mixture was stirred at room temperature for 12 h. The mixture was poured into water (150 mL). the aqueous mixture was extract 3 time with ethyl acetate (50 mL). The combined ethyl acetate fractions were washed with aqueous sodium bicarbonate, water, and brine. The organic fraction was dried (magnesium sulfate), filtered and concentrated. The product, ethyl 7-fluoro-1-methyl-1,4-dihydrochromene[4,3-c]pyrazole-3-carboxylate was purified on an ISCO system eluting with 5% ethyl acetate in methylene chloride. Yield 830 mg, 29%.

To a suspension of ethyl 7-fluoro-1-methyl-1,4-dihydrochromene[4,3-c]pyrazole-3-carboxylate (830 mg, 3.0 mmol) in methanol was added freashly powdered potassium hydroxide (337 mg, 6.0 mmol). The mixture was heated to reflux for 18 h. After cooling the mixture was diluted with water (30 mL) and cooled to 0° C. The pH was made acidic with 1N HCl. The solid, 7-fluoro-1-methyl-1,4-dihydrochromene[4,3-c]pyrazole-3-carboxylic acid was collected by filtration, washed with water and dried in a vacuum oven at 50° C. overnight. Yield 706 mg, 95% MS (ESI) m/z: Calculated for C₁₂H₉FN₂O₃: 248.1; found: 249.0 (M+1)⁺.

### 7-Fluoro-1-methyl-N-(6-methylpyridin-2-yl)-1,4-dihydrochromeno[4,3-c]pyrazole-3-carboxamide

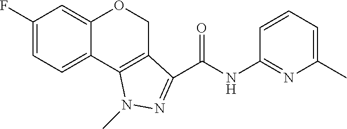

The title compound was prepared according to general procedure D described in Scheme 1. To a suspension of 7-fluoro-1-methyl-1,4-dihydrochromene[4,3-c]pyrazole-3-carboxylic acid (156 mg, 0.63 mmol) in methylene chloride (5 mL) was added a 2M (methylene chloride) solution of oxalyl chloride (1.6 mL, 3.15 mmol) followed by 1 drop of DMF. The resulting mixture was stirred at room temperature for 1 h. The mixture was concentrated under reduced pressure to provide a colorless solid. The solid was dissolved in methylene chloride (2 mL) and added dropwise to a solution of 2-amino-6-picoline (102 mg, 9.45 mmol) and triethylamine (263 uL, 1.9 mmol) in methylene chloride (3 mL). The resulting mixture was stirred at room temperature for 4 h. The mixture was diluted with ethyl acetate and washed with water and brine. The organic fraction was dried (magnesium sulfate), filtered and concentrated. The solid was repulped in ethyl acetate to provide the desired compound in 90% purity. The solid was recrystalized from ethyl acetate to provide the title compound (94 mg, 45% yield) in greater then 95% purity. ¹H NMR (300 MHz, CDCl₃): δ 9.19 (s, 1H), 8.10 (d, 1H), 7.61 (t, 1H), 7.46 (m,1H), 6.90 (d, 1H), 6.76 (m, 1H), 5.60 (s, 2H) 4.16 (s, 3H), 2.49 (s, 3H); MS (ESI) m/z: calculated for C₁₈H₁₅FN₄O₂: 338.1; found: 339.2 (M+1)⁺.

### Example 7

### N-(6-Methylpyridin-2-yl)-1,4-dihydrochromeno[4,3-c[pyrazole-3-carboxamide

### Ethyl 1,4-dihydrochromeno[4,3-c]pyrazole-3-carboxylate

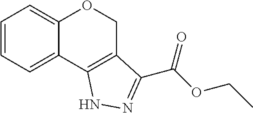

The title compound was prepared according to general procedure B described in Scheme 1. The title compound was obtained as a off-white solid (2.4 g, 9.9 mmol, 41% yield) by using ethyl 2-oxo-2-(4-oxochroman-3-yl)acetate (6.0 g, 24.17 mmol), hydrazine (0.83 ml, 26.59 mmol) and acetic acid (40 ml). MS (ESI) m/z: Calculated for C₁₃H₁₂N₂O₃: 244.1; found: 245.0 (M+H)⁺.

### 1,4-Dihydrochromeno[4,3-c]pyrazole-3-carboxylic acid

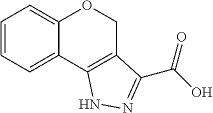

The title compound was prepared according to general procedure C described in Scheme 1. The title compound was obtained as a white solid (410 mg, 1.42 mmol, 81% yield) by using ethyl 1,4-dihydrochromeno[4,3-c]pyrazole-3-carboxylate (427 mg, 1.75 mmol), 1N potassium hydroxide in methanol (10.5 mL, 10.5 mmol), methanol (60 ml) and water (1 mL). MS (ESI) m/z: Calculated for C₁₁H₈N₂O₃: 216.1; found: 217.0 (M+H)⁺.

### N-(6-Methylpyridin-2-yl)-1,4-dihydrochromeno[4,3-c]pyrazole-3-carboxamide

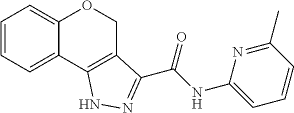

The title compound was prepared according to general procedure D described in Scheme 1. The title compound was obtained as a white solid with puritiy greater than 95% (1.6 mg, 0.005 mmol, 0.6% yield) by using 1,4-dihydrochromeno[4,3-c]pyrazole-3-carboxylic acid (190 mg, 0.88 mmol), oxalyl chloride (0.48 mL, 0.97 mmol), pyridine (0.21 ml, 2.6 mmol), 6-methylpyridin-2-amine (114 mg, 1.06 mmol) and dichloromethane (20 mL). ¹H NMR (300 MHz, DMSO): δ 7.97 (d, 1H), 7.74 (t, 1H), 7.63 (d, 1H), 7.26 (t, 1H), 7.02 (m, 3H), 5.57 (s, 2H), 2.44 (s, 3H); MS (ESI) m/z: Calculated for C₁₇H₁₄N₄O₂: 306.1; found: 307.2 (M+H)⁺.

### Example 8

### N-(4-Cyanopyridin-2-yl)-1-methyl-1,4-dihydrochromeno[4,3-c]pyrazole-3-carboxamide

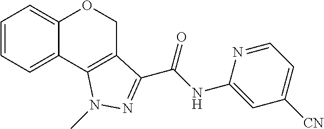

The title compound was prepared according to general procedure D described in Scheme 1. The title compound was obtained as a white solid with puritiy greater than 95% (38.7 mg, 0.12 mmol, 13.5% yield) by using 1-methyl-1,4-dihydrochromeno[4,3-c]pyrazole-3-carboxylic acid (200 mg, 0.87 mmol), oxalyl chloride (0.48 mL, 0.95 mmol), pyridine (0.21 ml, 2.6 mmol), 2-amino-4-cyanopyridine (124 mg, 1.04 mmol) and dichloromethane (20 mL). ¹H NMR (300 MHz, DMSO): δ 10.02 (br, 1H), 8.63 (d, 1H), 8.43 (s, 1H), 7.77 (d, 1H), 7.63 (d, 1H), 7.33 (t, 1H), 7.09 (m, 2H), 5.48 (s, 2H), 4.25 (s, 3H); MS (ESI) m/z: Calculated for C₁₈H₁₃N₅O₂: 331.1; found: 332.2 (M+H)⁻.

### Example 9

### N-(2,3-Dihydrobenzo[b][1,4]dioxin-6-yl)-1-methyl-1,4-dihydrochromeno[4,3-c]pyrazole-3-carboxamide

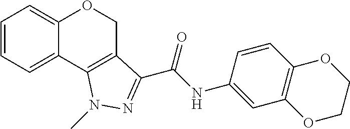

The title compound was prepared according to general procedure D described in Scheme 1. The title compound was obtained as a pale-yellow solid with puritiy greater than 95% (37.2 mg, 0.10 mmol, 15.7% yield) by using 1-methyl-1,4-dihydrochromeno[4,3-c]pyrazole-3-carboxylic acid (150 mg, 0.65 mmol), oxalyl chloride (0.36 mL, 0.72 mmol), pyridine (0.16 ml, 2.0 mmol), 3,4-ethylenedioxyaniline (118 mg, 0.78 mmol) and dichloromethane (15 mL). ¹H NMR (300 MHz, DMSO): δ 10.04 (s, 1H), 7.75 (dd, 1H), 7.42 (s, 1H), 7.29 (m, 2H), 7.05 (m, 2H), 6.79 (d, 1H), 5.45 (s, 2H), 4.22 (s, 7H); MS (ESI) m/z: Calculated for C₂₀H₁₇N₃O₄: 363.1; found: 364.2 (M+H)⁺.

### Example 10

### (2H-Benzo[b][1,4]oxazin-4(3H)-yl)(1-methyl-1,4-dihydrochromeno[4,3-c]pyrazol-3-yl)methanone

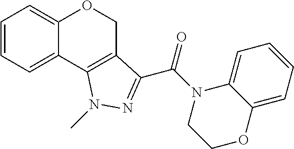

The title compound was prepared according to general procedure D described in Scheme 1. The title compound was obtained as a off-white solid with puritiy greater than 95% (42.7 mg, 0.12 mmol, 18.9% yield) by using 1-methyl-1,4-dihydrochromeno[4,3-c]pyrazole-3-carboxylic acid (150 mg, 0.65 mmol), oxalyl chloride (0.36 mL, 0.72 mmol), pyridine (0.16 ml, 2.0 mmol), 3,4-dihydro-3H-1,4-benzoxazine (105 mg, 0.78 mmol) and dichloromethane (15 mL). ¹H NMR (400 MHz, DMSO): δ 7.76 (d, 1H), 7.61 (br, 1H), 7.31 (dt, 1H), 7.12-7.02 (m, 3H), 6.91 (d, 1H), 6.84 (t, 1H), 5.26 (s, 2H), 4.33 (s, 4H), 4.18 (s, 3H); MS (ESI) m/z: Calculated for C₂₀H₁₇N₃O₃: 347.1; found: 348.2 (M+H)⁺.

### Example 11

### 5-Bromo-N-ethyl-N-((1-methyl-1,4-dihydrochromeno[4,3-c]pyrazol-3-yl)methyl)furan-2-carboxamide

The title compound was synthesized according to procedures in Scheme 3.

### (1-Methyl-1,4-dihydrochromeno[4,3-c]pyrazol-3-yl)methanol

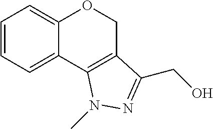

To a solution of ethyl 1-methyl-1,4-dihydrochromeno[4,3-c]pyrazole-3-carboxylate (300 mg, 1.16 mmol) in dichloromethane (20 mL) at −78° C. was added a solution of DIBAL (3.48 mL, 1M in dichloromethane) dropwise. The mixture was stirred at −78° C. for 1 h and was warmed up to room temperature and stirred overnight. LC-MS analysis indicated that the ester was converted to a mixture of the alcohol and the aldehyde that was the designed product for the next step. The reaction mixture was quenching at −78° C. with a saturated solution of NH₄Cl (0.5 mL) as well as 2N HCl (1.0 mL). The mixture was stirred at room temperature for 30 min. The mixture was extracted with dichloromethane and the extracts were dried over anhydrous sodium sulfate. The solution was filtered and the solvent was removed under reduced pressure to give the crude product (229 mg, a mixture with the aldehyde). The title compound was shown at the retention time 3.3 min in LC-MS analysis (Kromasil 100-3.5 C4 column, 50×4.6 mm, 3.5 μm. Mobile phase A was 0.05% TFA in water; mobile phase B was 0.05% TFA in acetonitrile. The gradient was initiated at 2% B and ramp to 75% B in 7 min, followed by a wash to 95% B within 0.5 min and re-equilibration back to 2% from 8.6 min. The total analyzed time was 10 min. Flow rate was 0.8 mL/min). MS (ESI) m/z: Calculated for C₁₂H₁₂N₂O₂: 216.1; found: 217.0 (M+H)⁺. The aldehyde was shown at the retention time 3.9 min in the same LC-MS analysis. MS (ESI) m/z: Calculated for C₁₂H₁₀N₂O₂: 214.1; found: 215.0(M+H)⁺.

### 1-Methyl-1,4-dihydrochromeno[4,3-c]pyrazole-3-carbaldehyde

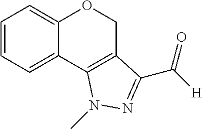

To the mixture of (1-methyl-1,4-dihydrochromeno[4,3-c]pyrazol-3-yl)methanol and the title aldehyde (229 mg, 1.06 mmol), NMO (149 mg, 1.27 mmol) and activated 4 Å molecular sieves (500 mg) in dichloromethane (50 mL) was added TPAP (37.2 mg, 0.11 mmol) at room temperature. After stirred overnight, the mixture was filtered and purified by silica gel column chromatography on an ISCO system (hexane/ethylacetate) to give a white solid (91 mg, 0.42 mmol, 40% yield). MS (ESI) m/z: Calculated for C₁₂H₁₀N₂O₂: 214.1; found: 215.0(M+H)⁺.

### N-((1-Methyl-1,4-dihydrochromeno[4,3-c]pyrazol-3-yl)methyl)ethanamine

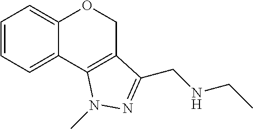

A mixture of 1-methyl-1,4-dihydrochromeno[4,3-c]pyrazole-3-carbaldehyde (91 mg, 0.42 mmol), acetic acid (40 μL) and ethylamine (0.84 mmol) in DCM/MeOH (5 mL/10 mL) was sonicated for 1 h. After the reaction mixture was cooled, sodium cyanoborohydride (26.7 mg, 0.425 mmol) was added and the reaction mixture was stirred at room temperature overnight. After the solvents were removed under reduced pressure, the crude product was obtained as a off-white solid. MS (ESI) m/z: Calculated for C₁₄H₁₇N₃O: 243.1; found: 244.2(M+H)⁺.

### 5-Bromo-N-ethyl-N-((1-methyl-1,4-dihydrochromeno[4,3-c]pyrazol-3-yl)methyl)furan-2-carboxamide

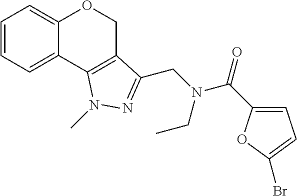

The title compound was obtained as a off-white gel with puritiy greater than 95% (9.0 mg, 0.02 mmol, 5.1% yield) by using 5-bromofuran-2-carboxylic acid (96 mg, 0.50 mmol), oxalyl chloride (0.27 mL, 0.55 mmol), pyridine (0.10 ml, 1.3 mmol), N-((1-methyl-1,4-dihydrochromeno[4,3-c]pyrazol-3-yl)methyl)ethanamine (102 mg, 0.42 mmol) and dichloromethane (10 mL). ¹H NMR (400 MHz, CDCl₃): δ 7.50 (d, 1H), 7.22 (t, 1H), 7.06-6.99 (m, 3H), 6.42 (d, 1H), 5.24 (br, 2H), 4.64 (br, 2H), 4.10 (s, 3H), 3.65 (br, 2H), 1.29 (br, 3H); MS (ESI) m/z: Calculated for C₁₉H₁₈BrN₃O₃: 415.1; found: 416.0 (M+H)⁺.

### Example 12

### 3-Chloro-N-((1-methyl-1,4-dihydrochromeno[4,3-c[pyrazol-3-yl)methyl)aniline

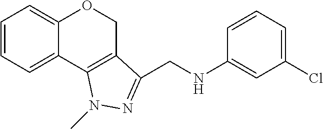

The title compound was synthesized according to procedure in Scheme 4. To a solution of N-(3-chlorophenyl)-1-methyl-1,4-dihydrochromeno[4,3-c]pyrazole-3-carboxamide (261 mg, 0.77 mM) in methylene chloride (5 mL) was added a 1M solution of DIBAL in methylene chloride (2.7 mL, 2.7 mM). The mixture was stirred at room temperature for 3 h. An additional 1.5 mL (1.5 mM) of DIBAL was added and the stirring was continued for 3 h. The reaction was quenched with 1N HCl (10 mL) and the mixture was stirred vigorously for 30 minutes. The organic fraction was separated, dried (sodium sulfate), filtered and concentrated. The residue was purified on an ISCO system eluting with 20% ethylacetate in hexanes. The product was then further purified by preparative TLC on silica gel eluting with 30% ethyl acetate in hexanes. Yield 20 mg, 8%. ¹H NMR (300 MHz, CDCl₃): δ 7.5 (dd, 1H), 7.21 (m, 1H), 7.05 (m, 3H), 6.67 (m, 2H), 6.53 (m, 1H), 5.09 (s, 2H), 4.27 (s, 2H), 4.11 (s, 3H); MS (ESI) m/z: calculated for C₁₈H₁₆ClN₃O: 325.10; found 326.3 (M+1)⁺.

### Example 13

### 1-Methyl-N-(6-methylpyridin-2-yl)-4,5-dihydro-1H-pyrazolo[4,3-h]quinoline-3-carboxamide

### Ethyl 1-methyl-4,5-dihydro-1H-pyrazolo[4,3-h]quinoline-3-carboxylate

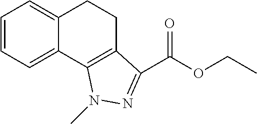

The title compound was prepared according to general procedures A and B described in Scheme 1. To a mixture of 6,7-dihydroquinolin-8(5H)-one (1 g, 6.71 mmol) in ethanol (3 mL) at 0° C. was added a solution of sodium ethoxide in ethanol (21 wt %, 2.75 mL, 7.38 mmol). The mixture was stirred 5 minutes at 0° C. and the treated with diethyl oxalate (909 uL, 6.781 mmol). The mixture was stirred at room temperature for 1 h and then recooled to 0° C. The reaction was quenched with 1N HCl in ethanol (7.4 mL). The solid was removed by filtration and the filtrate was concentrated to a dark oil under reduced pressure. The oil was dissolved in acetic acid (12 mL) and the solution was treated with methyl hydrazine (390 uL, 7.4 mmol). The solution was stirred 48 h at room temperature. The solid was removed by filtration. The filtrate was dissolved in ethyl acetate and the mixture was washed with water (3×20 mL), aqueous sodium bicarbonate and brine. The organic fraction was dried (MgSO₄), filtered and concentrated. The residue was purified by silica gel chromatography on an ISCO system eluting with a linear gradient for 5% ethyl acetate in metylene chloride to 30% ethyl acetate in methylene chloride. The major less polar regioisomer was identified as the desired compound. Yield 951 mg, 55%. ¹H NMR (300 MHz, CDCl₃): δ 8.45 (dd, 1H), 7.55 (dt, 1H), 7.13 (dd, 1H) 4.45 (m, 5H), 3.10 (m, 2H), 2.99 (m, 2H), 1.41 (t, 3H). Minor, more polar regioisomer 354 mg, 20% yield; ¹H NMR (300 MHz, CDCl₃): δ 8.51 (dd, 1H), 7.51 (dt, 1H), 7.12 (dd, 1H), 4.39 (q, 2H), 4.25 (s, 3H), 3.05 (m, 4H), 1.45 (t, 3H).

### 1-Methyl-4,5-dihydro-1H-pyrazolo[4,3-h]quinoline-3-carboxylic acid

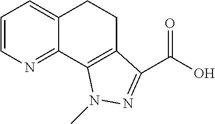

The title compound was prepared according to general procedure C described in Scheme 1. To ethyl 1-methyl-4,5-dihydro-1H-pyrazolo[4,3-h]quinoline-3-carboxylate (930 mg, 3.64 mmol) in tetrahydrofuran/methanol/water (20 mL, 3:1:1) was added lithium hydroxide (762 mg, 18.16 mmol). The mixture was heated to 50° C. for 1 h. After cooling the pH was adjusted to 4.0 with 1N HCl. The solid was collected by filtration and dried under high vacuum. Yield 666 mg, 80%. MS (ESI) m/z: calculated for C₁₂H₁₁N₃O₂: 229.1; found 230.00 (M+1)⁺.

### Methyl-N-(6-methylpyridin-2-yl)-4,5-dihydro-1H-pyrazolo[4,3-h]quinoline-3-carboxamide

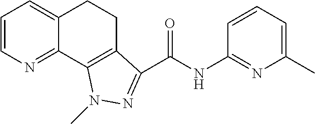

The title compound was prepared according to general procedure D described in Scheme 1. To a suspension of 1-methyl-4,5-dihydro-1H-pyrazolo[4,3-h]quinoline-3-carboxylic acid acid (200 mg, 0.88 mmol) in methylene chloride (6 mL) was added a 2M solution (methylene chloride) of oxalyl chloride (2.2 mL, 4.38 mmol) followed by 1 drop of DMF. The resulting mixture was stirred at room temperature for 1 h. The mixture was concentrated under reduced pressure to provide the acid chloride. The acid chloride was dissolved in methylene chloride (2 mL) and added dropwise to a solution of 2-amino-6-picoline (142 mg, 1.32 mmol) and triethylamine (367 uL, 2.64 mmol) in methylene chloride (4 mL). The resulting mixture was stirred at room temperature for 5 h. The mixture was diluted with ethyl acetate and washed with water and brine. The organic fraction was dried (magnesium sulfate), filtered and concentrated. The product was purified by silica gel chromatography on an ISCO system eluting with 10% ethyl acetate in methylene chloride. Yield 70 mg, 25%, ¹H NMR (300 MHz, CDCl₃): δ 11.8 (broad s, 1H), 8.71(d, 1H), 8.45 (dd, 1H), 8.06 (m, 1H), 7.56 (d, 1H), 7.14 (m, 2H), 4.5 (s, 3H), 3.14 (m, 2H), 3.03 (m, 2H), 2.83 (s, 3H); MS (ESI) m/z: calculated for C₁₈H₁₇N₅O: 319.1; found 320.2 (M+1)⁺.

### Example 14

### N-(3-Chlorophenyl)-1-methyl-4,5-dihydro-1H-benzo[g]indazole-3-carboxamide

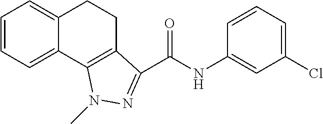

The title compound was prepared according to general procedure D described in Scheme 1. To a suspension of 1-methyl-4,5-dihydro-1H-benzo[g]indazole-3-carboxylic acid (228 mg, 1 mmol) in methylene chloride was added a 2M (methylene chloride) solution of oxalyl chloride (2 mL, 4 mmol) followed by 1 drop of DMF. The resulting mixture was stirred at room temperature for 2 h. The mixture was concentrated under reduced pressure to provide a colorless solid. The solid was dissolved in methylene chloride (2 mL) and added dropwise to a solution of 3-chloroaniline (153 mg, 1.2 mmol) and triethylamine (418 uL, 3 mmol) in methylene chloride (8 mL). The resulting mixture was stirred at room temperature for 4 h. The mixture was diluted with ethyl acetate and washed with 1N aq. HCl, water and brine. The organic fraction was dried (magnesium sulfate), filtered and concentrated. The residue was purified on an ISCO system eluting with 10% ethyl acetate in hexanes. Yield 220 mg. ¹H NMR (300 MHz, CDCl₃): 8.73 (s, 1H), 7.81 (s, 1H), 7.60 (m, 2H), 7.31 (m, 3H), 7.1 (m, 1H), 4.20 (s, 3H), 3.15 (m, 2H), 2.95 (m, 2H). MS (ESI) m/z calculated for C₁₉H₁₆ClN₃O: 337.1; found: 338.2 (M+H)⁺.

### Example 15

### 1-Isopropyl-N-(6-methylpyridin-2-yl)-4,5-dihydro-1H-pyrazolo[4,3-h[quinoline-3-carboxamide

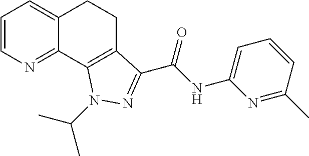

The title compound was prepared according to general procedure D described in Scheme 1. The title compound was obtained as a white solid with puritiy greater than 95% (88.0 mg, 0.25 mmol, 95% yield) by using 1-isopropyl-4,5-dihydro-1H-pyrazolo[4,3-h]quinoline-3-carboxylic acid (68.0 mg, 0.26 mmol), oxalyl chloride (7.5 mL, 92 mol), pyridine (0.55 ml, 6.8 mmol), 6-methylpyridin-2-amine (65.0 mg, 0.6 mmol) in dichloromethane (15 mL). ¹H NMR (400 MHz, CDCl₃): δ 9.31 (s, 1H), 8.44 (dd, 1H), 8.19 (d, 1H), 7.63 (d, 1H), 7.56 (t, 1H), 7.11 (q, 1H), 6.90 (d, 1H), 6.12 (m, 1H), 3.20 (dt, 2H), 2.99 (t, 2H), 2.52 (s, 3H), 1.56 (d, 6H); MS (ESI) m/z: Calculated for C₂₀H₂₁N₅O: 347.2; found: 348.2 (M+H)⁻

### Example 16

### 1-Ethyl-N-(6-methylpyridin-2-yl)-4,5-dihydro-1H-pyrazolo[4,3-h]quinoline-3-carboxamide

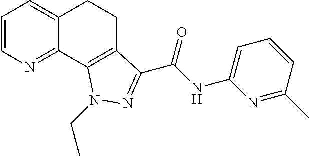

The title compound was prepared according to general procedure D described in Scheme 1. The title compound was obtained as a white solid with puritiy greater than 95% (76 mg, 0.23 mmol, 89% yield) by using 1-ethyl-4,5-dihydro-1H-pyrazolo[4,3-h]quinoline-3-carboxylic acid (62.0 mg, 0.25 mmol), oxalyl chloride (7.5 mL, 92 mmol), pyridine (0.45 ml, 5.6 mmol), 6-methylpyridin-2-amine (36.0 mg, 0.33 mmol) in dichloromethane (10 mL). ¹H NMR (400 MHz, CDCl₃): δ 9.34 (s, 1H), 8.44 (dd, 1H), 8.17 (d, 1H), 7.62 (d, 1H), 7.56 (t, 1H), 7.12 (dd, 1H), 6.90 (1H), 4.87 (q, 2H), 3.20 (dt, 2H), 3.01 (t, 2H), 2.50 (s, 3H), 1.52 (t, 3H); MS (ESI) m/z: Calculated for C₁₉H₁₉N₅O: 333.2; found: 334.2 (M+H)⁺

### Example 17

### 1-Methyl-N-(6-methylpyridin-2-yl)-4,5-dihydro-1H-pyrazolo[4,3-h]isoquinoline-3-carboxamide

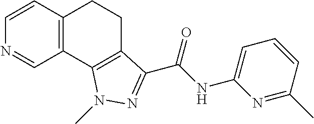

The title compound was prepared according to general procedure D described in Scheme 1. The title compound was obtained as a white solid with puritiy greater than 90% (10.3 mg, 0.032 mmol, 17% yield) by using 1-methyl-4,5-dihydro-1H-pyrazolo[4,3-h]isoquinoline-3-carboxylic acid (60.0 mg, 0.22 mmol), oxalyl chloride (85.0 mg, 0.68 mmol), triethylamine (88.0 ml, 0.63 mmol), 6-methylpyridin-2-amine (27.0 mg, 0.025 mmol) in dichloromethane (6.0 mL). ¹H NMR (400 MHz, CDCl₃): δ 9.28 (s, 1H), 8.65 (s, 1H), 8.48 (d, 1H), 8.14 (d, 1H), 7.62 (t, 1H), 6.91 (d, 1H), 4.24 (s, 3H), 3.18 (dt 2H), 2.98 (t, 2H), 2.50 (s, 3H); MS (ESI) m/z: Calculated for C₁₈H₁₇N₅O: 319.1; found: 320.2 (M+H)⁺

### Example 18

### 1,8-Dimethyl-N-(6-methylpyridin-2-yl)-4,5-dihydro-1H-pyrazolo[4,3-h[quinoline-3-carboxamide

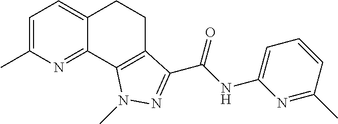

The title compound was prepared according to procedures described in Scheme 5. The title compound was obtained as a white solid with puritiy greater than 90%: ¹H NMR (400 MHz, CDCl₃): δ 3.32 (s, 1H), 8.15 (d, 1H), 7.60 (t, 1H), 7.43 (d, 1H), 6.98 (d, 1H), 6.89 (d, 1H), 4.41 (s, 3H), 3.17 (t, 2H), 2.96 (t, 2H), 2.54 (s, 3H), 2.49 (s, 3H); MS (ESI) m/z: Calculated for C₁₉H₁₉N₅O: 333.2; found: 334.1 (M+H)⁺.

### Example 19

### 5-Acetyl-N-(3-chlorophenyl)-1-methyl-4,5-dihydro-1H-pyrazolo[4,3-c[quinoline-3-carboxamide

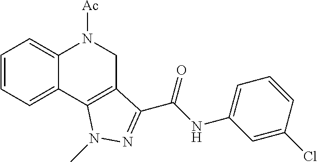

The title compound was prepared according to general procedures A-D described in Scheme 6 (20 mg, 13.2% yield): ¹H NMR (300 MHz, CD₃OD): δ 8.57 (s, 1H), 7.76 (t, 1H), 7.57 (d, 2H), 7.47 (d, 1H), 7.36-7.26 (m, 3H), 7.04 (dd, 1H), 5.10 (s, 2H), 4.18 (s, 3H), 2.21 (s, 3H); MS (ESI) m/z: Calculated for C₂₀H₁₇ClN₄O₂: 380.8; found: 381.0 (M+H)⁺.

### Example 20

### N-(3-Chlorophenyl)-1-methyl-1H-pyrazolo[4,3-c]quinoline-3-carboxamide

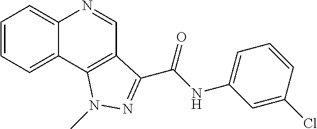

The title compound was prepared as described in step 6 of Scheme 6: 5-acetyl-N-(3-chlorophenyl)-1-methyl-4,5-dihydro-1H-pyrazolo[4,3-c]quinoline-3-carboxamide (60 mg, 0.16 mmol) was mixed with 6 N HCl (2 mL, 0.33 mmol) in a mixture of water (2 mL) and MeOH (5 mL). The mixture was heated at 120° C. for 30 min under microwave irradiation (Personal Chemistry Emrys™ Optimizer microwave reactor). Upon completion of the reaction, the reaction mixture was concentrated under reduced pressure. The crude product was purified by reverse phase preparative HPLC to yield the title compound as trifluoroacetic salt with puritiy greater than 95% (13 mg, 18% yield): ¹H NMR (400 MHz, DMSO-d₆): δ 10.81 (s, 1H), 9.59 (s, 1H), 8.61 (d, 1H), 8.22 (d, 1H), 8.11 (s, 1H), 7.86 (m, 2H), 7.80 (t, 1H), 7.39 (t, 1H), 7.19 (d, 1H), 4.62 (s, 3H); MS (ESI) m/z: Calculated for C₁₈H₁₃ClN₄O: 336.1; found: 337.2 (M+H)⁺.

### Example 21

### N-(3-Chlorophenyl)-1-methyl-4,5-dihydro-1H-pyrazolo[4,3-c]quinoline-3-carboxamide

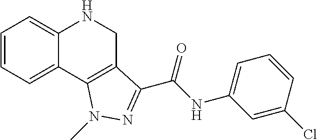

The title compound was prepared as described in step 7 of Scheme 6: N-(3-chlorophenyl)-1-methyl-1H-pyrazolo[4,3-c]quinoline-3-carboxamide (21 mg, 0.063 mmol) was treated with NaBH₄ (24 mg, 0.63 mmol) in a mixture of THF (3 mL) and ethanol (0.8 mL). The mixture was stirred at room temperature overnight. The solvent was removed under reduced pressure. The crude product was purified by reverse phase preparative HPLC to yield the title compound as trifluoroacetic salt with puritiy greater than 95% (2.2 mg, 10% yield): ¹H NMR (400 MHz, CD₃OD): δ 7.88 (dd, 1H), 7.56 (d, 1H), 7.54 (d, 1H), 7.32 (t, 1H), 7.11 (d, 1H), 7.06 (t, 1H), 6.71 (m, 2H), 4.71 (s, 2H), 4.19 (s, 3H); MS (ESI) m/z: Calculated for C₁₈H₁₅ClN₄O: 338.1; found: 339.0 (M+H)⁺.

### Example 22

### 3-Methyl-N-(6-methylpyridin-2-yl)-3,4,14-triazatricyclo[8.4.0.0̂{2,6}]tetradeca-1(14),2(6),4,10,12-pentaene-5-carboxamide

The title compound was synthesized according to Scheme 7.

### 6,7,8,9-Tetrahydro-5H-cyclohepta[b]pyridine-1-oxide

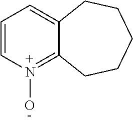

To a solution of 6,7,8,9-tetrahydro-5H-cyclohepta[b]pyridine (10 g) in DCM (100 mL), was added 60% mCPBA (20.2 g) with ice cooling. After being stirred for 2 h at room temperature, the reaction was washed with saturated NaHCO₃, and brine. The organic layer was dried over Na₂SO₄, concentrated, and then purified by silica chromatography (5 to 10% MeOH/DCM) to yield 7.3 g of the title compound in 66% yield.

### 6,7,8,9-Tetrahydro-5H-cyclohepta[b]pyridin-9-ol

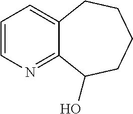

To a solution of 6,7,8,9-tetrahydro-5H-cyclohepta[b]pyridine-1-oxide 2 (7.3 g) in DCM (40 mL), was added 20 ml of TFAA with ice cooling. The reaction was stirred for 4 h at room temperature. To this, was added of saturated K₂CO₃ until basic. After being stirred for 3 h at rt, the reaction mixture was extracted DCM (×2) and washed with brine. The combined organic layers were dried over Na₂SO₄, concentrated, and then purified by silica chromatography (30% EtOAc in hexanes) to yield 3.0 g of the title compound in 38% yield.

### 5,6,7,8-Tetrahydrocyclohepta[b]pyridin-9-one

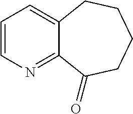

To a solution of 6,7,8,9-tetrahydro-5H-cyclohepta[b]pyridin-9-ol (3.0 g) and 1.9 mL of DMSO in DCM (50 mL), was added dropwise 2.4 mL of oxalyl chloride at −78° C. After 1 h, 8.6 mL of triethylamine was added slowly and then stirred for an additional 30 min. The reaction was stirred at room temperature for 2 h, it was then extracted with DCM and washed with brine. The organic layer was dried over Na₂SO₄, concentrated, and then purified by silica chromatography to yield 2.6 g of the title compound in 89% yield.

### 2-(6,7,8,9-Tetrahydro-9-oxo-5H-cyclohepta[b]pyridin-8-yl)-2-oxoacetic acid

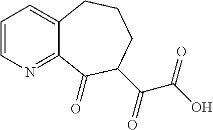

To a freshly prepared NaOEt solution (0.8 g of Na in 40 mL of absolute EtOH) was added 2.7 mL of diethyl oxalate followed by a solution of 5,6,7,8-tetrahydrocyclohepta[b]pyridin-9-one (2.6 g in 6 mL of absolute EtOH) in a water bath. The reaction was stirred at room temperature for 1 h and then quenched by addition of saturated citric acid solution until acidic (pH=3-4). The mixture was extracted with EtOAc (×2), washed with brine, dried over Na₂SO₄, and then concentrated to yield 2.0 g of the title compound (53%).

### 3-Methyl-3,4,14-triazatricyclo[8.4.0.0̂{2,6}]tetradeca-1(14),2(6),4,10,12-pentaene-5carboxylic acid

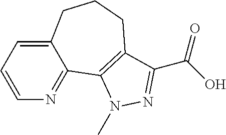

To a mixture of 2-(6,7,8,9-tetrahydro-9-oxo-5H-cyclohepta[b]pyridin-8-yl)-2-oxoacetic acid (1.1 g, 4.9 mmol) in 25 mL of glacial acetic acid, was added 0.36 mL of methylhydrazine at room temperature. The volatiles were removed in vacuo. To the resulting suspension, was added 30 mL of EtOAc and then it was stirred for 1 h at room temperature. The resulting solids were collected by filtration, washed with EtOAc and then dried to yield 1.1 g of title compound (92%) as a yellow solid; ¹H NMR (400 MHz, CDCl₃): δ 8.60 (dd, 1H), 7.62 (m, 1H), 7.24 (m, 1H), 4.23 (s, 3H), 3.04 (m, 2H), 2.68 (m, 2H), 2.13 (m, 2H); MS (ESI) m/z: Calculated for C₁₃H₁₃N₃O₂: 243.1; found: 244.1 (M+H)⁻.

### 3-Methyl-N-(6-methylpyridin-2-yl)-3,4,14-triazatricyclo[8.4.0.0̂{2,6}]tetradeca-1(14),2(6),4,10,12-pentaene-5-carboxamide

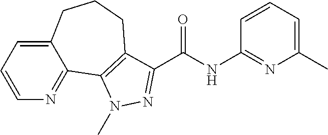

A suspension of 3-methyl-3,4,14-triazatricyclo[8.4.0.0̂{2,6}]tetradeca-1(14),2(6),4,10,12-pentaene-5carboxylic acid (130 mg, 0.53 mmol) and 10 μL of DMF in anhydrous DCM (5 mL), was added 130 uL of oxalyl chloride at room temperature. After being stirred for 1 h, 1 mL of triethylamine was added to the reaction mixture. To this was added a solution of 150 mg of 2-amino-6-methylpyridine in anhydrous DCM (3 mL). After stirring for 1 h the mixture was extracted with EtOAc (×2), washed with 1N NaOH followed by brine, dried over Na₂SO₄, and then purified by PTLC (40% EtOAc/hexane) to yield 25 mg (14%) of the title compound as a brown solid. ¹H NMR (400 MHz, CDCl₃): δ 10.22 (s, 1H), 8.62 (dd, 1H), 8.09 (d, 1H), 7.98 (t, 1H), 7.83 (dd, 1H), 7.39 (dd, 1H), 7.20 (d, 1H), 4.20 (s, 3H), 2.98 (dd, 2H), 2.65 (m, 2H), 2.52 (s, 3H), 2.06(m, 2H); MS (ESI) m/z: Calculated for C₁₉H₁₉N₅O: 333.2; found: 334.2 (M+H)⁺.

### Example 23

### 4-Methyl-N-(6-methylpyridin-2-yl)-3,4,14-triazatricyclo[8.4.0.0̂{2,6}]tetradeca-1(14),2,5,10,12-pentaene-5-carboxamide

The title compound was synthesized according to Scheme 7.

### 4-Methyl-3,4,14-triazatricyclo[8.4.0.0̂{2,6}]tetradeca-1(14),2,5,10,12-pentaene-5-carboxylic acid

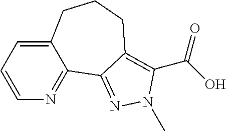

The title compound was obtained as a white solid (11 mg, 3% yield) by using 2-oxo-2-(9-oxo-6,7,8,9-tetrahydro-5H-cyclohepta[b]pyridin-8-yl)acetic acid (400 mg, 1.72 mmol), methyl hydrazine (87 mg, 1.89 mmol) and acetic acid (4 ml). Purification conditions were as follows (water/acetonitrile (0.05% TFA); 5-50% acetonitrile over 45 min. The title compound was shown at the retention time 2.84 min in LC-MS analysis (Kromasil 100-3.5 C4 column, 50×4.6 mm, 3.5 μm. Mobile phase A was 0.05% TFA in water; mobile phase B was 0.05% TFA in acetonitrile. The gradient was initiated at 2% B and ramp to 75% B in 7 min, followed by a wash to 95% B within 0.5 min and re-equilibration back to 2% from 8.6 min. The total analyzed time was 10 min. Flow rate was 0.8 mL/min). ¹H NMR (400 MHz, CDCl₃): δ 8.87 (m, 1H), 8.12 (m, 1H), 7.65 (m, 1H), 4.19 (s, 3H), 3.20 (m, 2H), 3.07 (m, 2H), 2.13 (m, 2H); Calculated for C₁₃H₁₃N₃O₂: 243.1; found: 244.3 (M+H)⁺.

### 4-Methyl-N-(6-methylpyridin-2-yl)-3,4,14-triazatricyclo[8.4.0.0̂{2,6}]tetradeca-1(14),2,5,10,12-pentaene-5-carboxamide

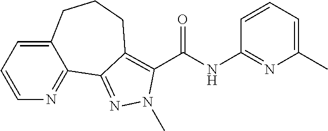

The title compound was obtained as a white solid with puritiy greater than 95% (4.2 mg, 31% yield) by using 4-methyl-3,4,14-triazatricyclo[8.4.0.0̂{2,6}]tetradeca-1(14),2(6),4,10,12-pentaene-5carboxylic acid (10 mg, 0.04 mmol), 6-methylpyridin-2-amine (8.9 mg, 0.08 mmol), diisopropylethylamine (0.044 ml, 0.24 mmol), N-(3-dimethylaminopropyl)-N′-ethylcarbodiimide (EDC, 22.9 mg, 0.12 mmol), HOBt (16.2 mg, 0.12 mmol) in dichloromethane (2 mL). MS (ESI) m/z: Calculated for C₁₉H₁₉N₅O: 333.2; found: 334.4 (M+H)⁻.

### Example 24

### (4-Fluorophenyl)(3-(2-methyl-4,5-dihydro-2H-benzo[g]indazol-3-yl)piperidin-1-yl)methanone

The title compound was synthesized according to Scheme 8.

### Ethyl 1-(4-fluorobenzoyl)piperidine-3-carboxylate

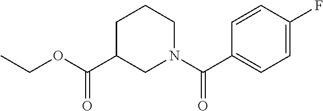

To a solution of ethyl nipecotate (10 g, 63.6 mmol) and DIPEA (22.2 mL, 127.2 mmol) in dichloromethane (300 mL) was added 4-fluorobenzoyl chloride (8.01 mL, 66.8 mmol) slowly at room temperature. The mixture was stirred for 4 h and then the mixture was washed with 1N NaOH and 1N HCl. The organic phase was separated and dried over anhydrous sodium sulfate. The solvent was removed under reduced pressure to give a pale yellow oil (17.1 g, 61.2 mmol, 97%). MS (ESI) m/z: Calculated for C₁₅H₁₈FNO₃: 279.1; found: 280.2 (M+H)⁺.

### 1-(4-Fluorobenzoyl)piperidine-3-carboxylic acid

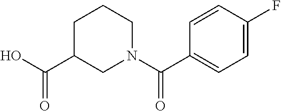

The title compound was prepared according to general procedure C described in Scheme 8. The title compound was obtained as an off-white solid by using ethyl 1-(4-fluorobenzoyl)piperidine-3-carboxylate (36.7 mmol), 1N potassium hydroxide in methanol (200 mL, 200 mmol), methanol (200 ml) and water (5 mL). MS (ESI) m/z: Calculated for C₁₃H₁₄FNO₃: 251.1; found: 252.0 (M+H)⁺.

### 2-(1-(4-Fluorobenzoyl)piperidine-3-carbonyl)-3,4-dihydronaphthalen-1(2H)-one

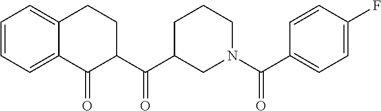

The title compound was prepared according to steps 3 and 4 described in Scheme 8. To 1M a solution of lithium hexamethyldisilazide in THF (12.5 mL, 12.5 mmol) was added α-tetralone (1.66 g, 1.52 mL, 11.38 mmol) in THF (12 mL) at −78° C. under argon atmosphere, the resulting mixture was stirred for 30 min at the same temperature (solution A). A solution of 1,1′-carbonyldi-1H-imidazole (CDI, 1.88 g, 11.38 mmol) and 1-(4-fluorobenzoyl)piperidine-3-carboxylic acid (2.86 g, 11.38 mmol) in THF (35 mL) was stirred for 45 min at room temperature (solution B). The resulting solution B was added dropwise to solution A over 30 min at −78° C. After removal of the cold bath, the reaction mixture was warmed to room temperature and stirred for 12 h. To this resulting mixture was added 10% aqueous citric acid (35 mL), and the resulting solution was taken up in ethylacetate, washed with brine, dried over anhydrous sodium sulfate, and concentrated to dryness. The residue was purified by silica gel column chromatography on an ISCO system to yield a pale yellow sticky oil (420 mg, 1.11 mmol, 9.7% yield). MS (ESI) m/z: Calculated for C₂₃H₂₂FNO₃: 379.2; found: 380.1 (M+H)⁺.

### Example 25

### (4-Fluorophenyl)(3-(2-methyl-4,5-dihydro-2H-benzo[g]indazol-3-yl)piperidin-1-yl)methanone

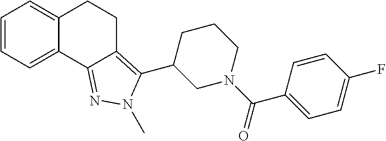

The title compound was prepared according to general procedure B as described in step 5 in Scheme 8. A mixture of 2 regio-isomers were obtained by using 2-(1-(4-fluorobenzoyl)piperidine-3-carbonyl)-3,4-dihydronaphthalen-1(2H)-one (100 mg, 0.26 mmol), methylhydrazine (15.3 μL, 0.29 mmol), acetic acid (0.5 ml) and dichloromethane (5 mL). The title compound was isolated by prep-HPLC (1% TFA/acetonitril/water) to yield a TFA salt as a white solid (11.0 mg, 0.028 mmol, 10.9% yield). The title compound was shown at the retention time 16.2 min in LC-MS analysis (Phenomenex Luna C18 column, 100×2.0 mm, 5 μm. Mobile phase A was 0.05% TFA in water; mobile phase B was 0.05% TFA in acetonitrile. The gradient was initiated at 5% B for 1 min and then ramp to 75% B in 23 min, followed by a wash to 95% B within 1 min and re-equilibration back to 2% from 27 min. The total analyzed time was 30 min. Flow rate was 0.8 mL/min). ¹H NMR (400 MHz, CD₃OD): δ 7.64 (br, 1H), 7.48 (br, 2H), 7.38-7.1 (m & br, 5H), 4.70 (br, 1H), 4.52 (br, 1H), 4.07 (d, 3H), 3.75 (d, 1H), 3.20-2.68 (m & br, 4H), 2.51 (br, 1H), 2.30(br, 1H), 2.10 (d, 1H), 1.94 (br, 1H), 1.80-1.60 (br, 2H); ¹⁹F NMR (376 MHz, CDCl₃): δ −112.49; MS (ESI) m/z: Calculated for C₂₄H₂₄FN₃O: 389.2; found: 390.2 (M+H)⁺.

### Example 25

### (4-Fluorophenyl)(3-(1-methyl-4,5-dihydro-1H-benzo[g]indazol-3-yl)piperidin-1-yl)methanone

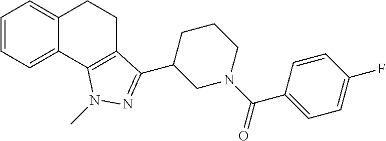

The title compound was prepared according to general procedure B as described in step 5 in Scheme 8. A mixture of 2 regio-isomers were obtained by using 2-(1-(4-fluorobenzoyl)piperidine-3-carbonyl)-3,4-dihydronaphthalen-1(2H)-one (100 mg, 0.26 mmol), methylhydrazine (15.3 μL, 0.29 mmol), acetic acid (0.5 ml) and dichloromethane (5 mL). The title compound was isolated by prep-HPLC (1% TFA/acetonitril/water) to yield a TFA salt as a white solid (8.1 mg, 0.028 mmol, 8.0% yield). The title compound was shown at the retention time 15.2 min in LC-MS analysis (Phenomenex Luna C18 column, 100×2.0 mm, 5 μm. Mobile phase A was 0.05% TFA in water; mobile phase B was 0.05% TFA in acetonitrile. The gradient was initiated at 5% B for 1 min and then ramp to 75% B in 23 min, followed by a wash to 95% B within 1 min and re-equilibration back to 2% from 27 min. The total analyzed time was 30 min. Flow rate was 0.8 mL/min). ¹H NMR (400 MHz, CD₃OD): δ 7.68 (br, 1H), 7.52 (m, 2H), 7.22 (t, 5H), 4.69 (br, 1H), 3.94 (br, 1H), 3.85-3.6 (br, 2H), 3.40-3.20 (br, 1H), 3.10 (br, 2H), 2.90 (br, 5H), 2.08 (br, 2H), 1.80-1.6 (br, 2H); ¹⁹F NMR (376 MHz, CDCl₃): δ −112.49; MS (ESI) m/z: Calculated for C₂₄H₂₄FN₃O: 389.2; found: 390.2 (M+H)⁺.

### Example 26

### mGluR5 Calcium Mobilization in Inducible mGluR5 CHO Cells

To functionally measure mGluR5 calcium mobilization in response to agonists or antagonists, an inducible CHO cell line expressing the human mGlu5 recombinant receptor was used. This cell line also stably expressed Aequorin (Euroscreen FAST), a photoprotein that spontaneously couples with coelenterazine h when added exogenously in the cell media. The Aequorin-coelenterzine h complex undergoes a conformational change upon binding of calcium ions which results in the emission of blue light (wavelength 469 nm) that was measured using a Hamamatsu Functional Drug Screening System 6000.

mGluR5 expression was induced by a doxycycline responsive promoter by growing cells to mid-log phase for 18 hours in media without antibiotics and supplemented with doxycycline (600 ng/mL). Cells were detached from the plate by gentle flushing using PBS-EDTA (5 mM EDTA) and centrifuged for collection and resuspended in “assay buffer” (HBSS, 2.1 mM CaCl2, 2 ug/mL glutamate-pyruvate transaminase, 4 mM MEM sodium pyruvate, and 0.1% protease free BSA). Cells were incubated with coelenterazine h (Molecular Probes) at room temperature for 4 hours before testing.

To measure mGluR5 antagonist activity, cell suspensions containing test compound were mixed with EC80 glutamate and emission of light was recorded. Reference and test compounds were tested at several concentrations in duplicate to obtain dose response curves using the Prism GraphPad software (GraphPad Software Inc.). IC50 values were calculated from the dose response curves using the equation:

Y=Bottom+(Top−Bottom)/(1+10̂((LogEC50−X)*HillSlope)).

The results are given in Tables 2 and 3.

TABLE 2

| | | IC₅₀ |
|---|---|---|
| Ex- | | (uM) |
| ample | | Ca⁺⁺ |
| No. | Structure | flux ^{a} |
| 1 | 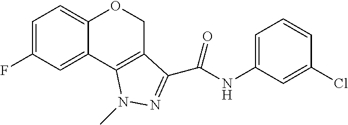 | +++ |
| 2 | 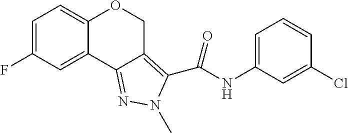 | +++ |
| 3 | 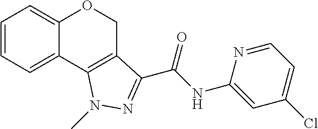 | ++ |
| 4 | 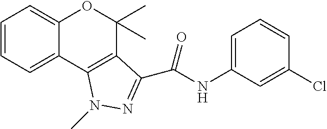 | + |
| 5 | 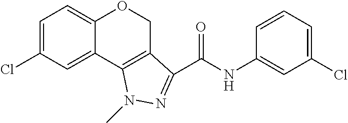 | + |
| 6 | 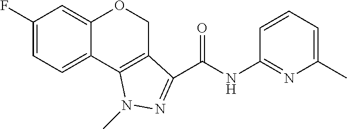 | + |
| 7 | 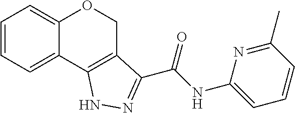 | + |
| 8 | 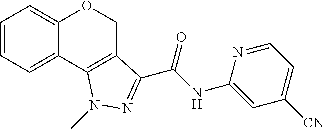 | + |
| 9 | 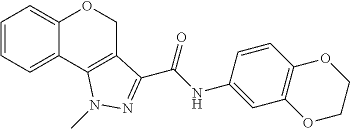 | + |
| 10 | 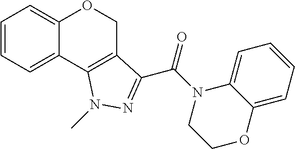 | + |
| 11 | 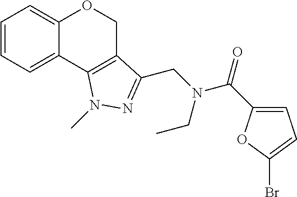 | + |
| 12 | 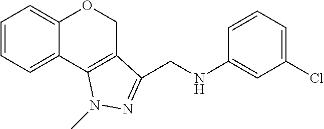 | ++ |
| 13 | 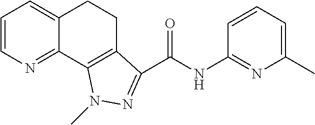 | +++ |
| 14 | 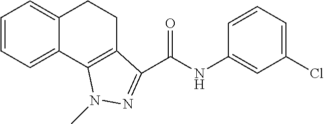 | +++ |
| 15 | 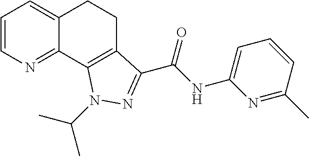 | + |
| 16 | 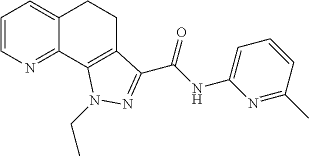 | +++ |
| 17 | 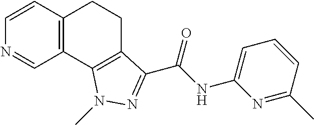 | +++ |
| 18 | 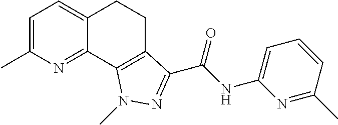 | +++ |
| 19 | 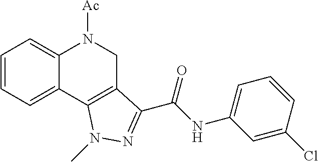 | +++ |
| 20 | 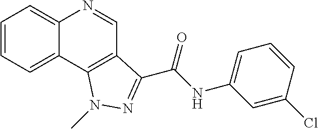 | +++ |
| 21 | 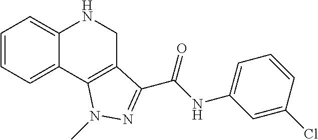 | +++ |
| 22 | 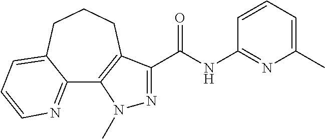 | +++ |
| 23 | 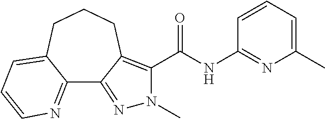 | + |
| 24 | 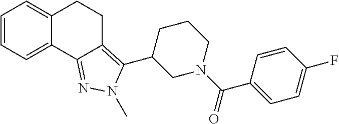 | + |
| 25 | 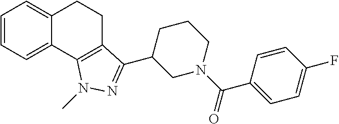 | + |

^{a} when IC₅₀ > 10 uM: designated as “+”; when 10 uM > IC₅₀ > 1 uM; designated as “++”; when IC₅₀ < 1 uM, designated as “+++”.

TABLE 3

| | IC₅₀ (uM) |
|---|---|
| Compound | Ca⁺⁺ flux ^{a} |
| N-(3-chlorophenyl)-N-methyl-4H- | + |
| chromeno[3,4-d]isoxazole-3- | |
| carboxamide | |
| 1-methyl-N-(3-(trifluoromethyl)phenyl)- | + |
| 1,4-dihydrochromeno[4,3-c]pyrazole-3- | |
| carboxamide | |
| N-(3-chlorophenyl)-N,1-dimethyl-1,4- | + |
| dihydrochromeno[4,3-c]pyrazole-3- | |
| carboxamide | |
| N-(2-chlorophenyl)-1-methyl-1,4- | ++ |
| dihydrochromeno[4,3-c]pyrazole-3- | |
| carboxamide | |
| N-(4-chlorophenyl)-1-methyl-1,4- | + |
| dihydrochromeno[4,3-c]pyrazole-3- | |
| carboxamide | |
| N-(4-chlorophenyl)-4,5-dihydro-1H- | + |
| benzo[g]indazole-3-carboxamide | |
| N-(pyridin-3-yl)-1,4- | + |
| dihydrochromeno[4,3-c]pyrazole-3- | |
| carboxamide | |
| N-(3-methoxyphenyl)-1,4- | + |
| dihydrochromeno[4,3-c]pyrazole-3- | |
| carboxamide | |
| N-(4-methoxyphenyl)-1,4- | + |
| dihydrochromeno[4,3-c]pyrazole-3- | |
| carboxamide | |
| N-(3-chlorophenyl)-1,8-dimethyl-1,4- | +++ |
| dihydrothiochromeno[4,3-c]pyrazole-3- | |
| carboxamide | |
| N-(4-fluorophenyl)-1-methyl-1,4- | + |
| dihydrochromeno[4,3-c]pyrazole-3- | |
| carboxamide | |
| N-(3-chloro-4-fluorophenyl)-1-methyl- | + |
| 1,4-dihydrochromeno[4,3-c]pyrazole-3- | |
| carboxamide | |
| N-(5-chloro-2-methoxyphenyl)-1- | ++ |
| methyl-1,4-dihydrochromeno[4,3- | |
| c]pyrazole-3-carboxamide | |
| N-(3-methoxyphenyl)-1-methyl-1,4- | + |
| dihydrochromeno[4,3-c]pyrazole-3- | |
| carboxamide | |
| N-(5-chloropyridin-2-yl)-1-methyl-1,4- | +++ |
| dihydrochromeno[4,3-c]pyrazole-3- | |
| carboxamide | |
| N-(3-chlorophenyl)-1-methyl-1,4- | +++ |
| dihydrothiochromeno[4,3-c]pyrazole-3- | |
| carboxamide | |
| N-(3-chlorobenzyl)-1-methyl-1,4- | + |
| dihydrochromeno[4,3-c]pyrazole-3- | |
| carboxamide | |
| N-(3-chlorophenyl)-1-methyl-4,5- | + |
| dihydro-1H-thieno[2,3-g]indazole-3- | |
| carboxamide | |
| N-(3-chlorophenyl)-4H- | +++ |
| thiochromeno[3,4-d]isoxazole-3- | |
| carboxamide | |

^{a} when IC₅₀ > 10 uM: designated as “+”; when 10 uM > IC₅₀ > 1 uM; designated as “++”; when IC₅₀ < 1 uM, designated as “+++”.

### REFERENCES

All publications and patents mentioned herein, including those items listed below, are hereby incorporated by reference in their entirety as if each individual publication or patent was specifically and individually incorporated by reference. In case of conflict, the present application, including any definitions herein, will control.

### Equivalents

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

1-93. (canceled)

94. A compound of formula VII: 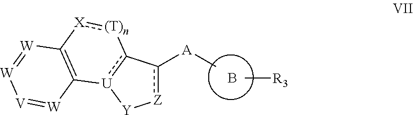
or a pharmaceutically acceptable salt or N-oxide thereof, wherein:
each W is independently N or CR₁;
V is N or CR₂;
X is selected from the group consisting of: O, S, SO₂, N, NR₄, CR₅, CR₅R₆, and CO;
each T is independently selected from the group consisting of: CO, CR₇R_{8,} and NR₉;
U is selected from the group consisting of: C, CR₁₀, and N;
Y is selected from the group consisting of: N, NR₁₁, O, and CR₁₂;
Z is selected from the group consisting of: N, NR₁₃, and O;
A is selected from the group consisting of: —CONR₁₆—, —NR₁₆CO—, —O—, —CONR₁₆—CR₁₄R₁₅—, —NR₁₆CONR₁₆—, —CR₁₄R₁₅—NR₁₆CO—, —CR₁₄R₁₅—O—, —CR₁₄R₁₅—NR₁₆—, —O—CR₁₄R₁₅—, —NR₁₆—CR₁₄R₁₅—, —NR₁₆—, 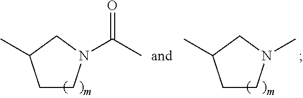
B is selected from the group consisting of: aryl, heteroaryl, and heterocyclyl; wherein B is optionally substituted by one, two, or three substituents each independently represented by R₃;
n is 0, 1 or 2;
m is 0, 1 or 2;
R₁, R₂, and R₃ are each independently selected from the group consisting of: alkoxy, alkyl, alkenyl, alkynyl, alkanoyl, amino, aryl, carboxamido, cycloalkyl, cyano, halogen, haloalkyl, heteroaryl, heterocyclyl, hydrogen, hydroxyl, nitro, sulfonamido, sulfonyl, and thio;
R₄, R₉, R₁₁, R₁₃, and R₁₆ are each independently selected from the group consisting of: alkyl, alkylsulfonyl, alkanoyl, cycloalkyl, haloalkyl, and hydrogen;
R₅, R₆, R₁₂, R₁₄, and R₁₅ are each independently selected from the group consisting of: alkoxy, alkyl, alkanoyl, amino, aryl, cycloalkyl, cyano, halogen, haloalkyl, hydrogen, and hydroxyl;
R₇ and R₈ are each independently selected from the group consisting of: alkoxy, alkyl, halogen, haloalkyl, hydrogen, and hydroxyl, or R₇ and R₈ taken together are oxo; or R₇ and R₈ taken together form a carbocycle or a heterocycle; and
R₁₀ is selected from the group consisting of: methyl, oxide, or hydrogen.

95. A method of treating Alzheimer's disease, Fragile X syndrome, L-DOPA induced dyskinesia, depression, anxiety, migraine, pain, gastroesophageal reflux disease, and/or aiding smoking cessation comprising administering to a subject in need thereof a therapeutically effective amount of a compound according to claim 94.

96. A compound defined in claim 94 represented by formula I: 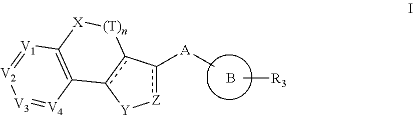
and pharmaceutically acceptable salts or N-oxides thereof; wherein
V₁, V₂, V₃, and V₄ are selected, independently for each occurrence, from the group consisting of: N and CR₂;
X is NR₄ or CR₇R₈;
Y and Z are each independently selected from the group consisting of: NR₁₁ and N;
T is CO or CR₇R₈, wherein at least one T is CR₇R_{8;}
n is 0, 1 or 2;
A is selected from the group consisting of: —CONR₁₆—, —NR₁₆CO—, —O—, —NR₁₆CONR₁₆—, —CR₁₄R₁₅—NR₁₆CO—, —CR₁₄R₁₅—O—, —CR₁₄R₁₅—NR₁₆—, —O—CR₁₄R₁₅—, —NR₁₆—CR₁₄R₁₅—, —NR₁₆—, 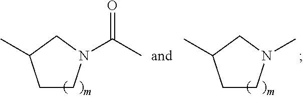
m is 0, 1 or 2;
R₂ is independently selected for each occurrence from the group consisting of: alkoxy, alkyl, alkenyl, alkynyl, alkanoyl, amino, aryl, carboxamido, cycloalkyl, cyano, halogen, haloalkyl, heteroaryl, heterocyclyl, hydrogen, hydroxyl, nitro, sulfonamido, sulfonyl, and thio;
B is selected from the group consisting of: aryl, heteroaryl, and heterocyclyl, wherein B is optionally substituted by one, two, or three substituents each independently represented by R₃;
R₃ is selected from the group consisting of: alkoxy, alkyl, alkenyl, alkynyl, alkanoyl, amino, aryl, carboxamido, cycloalkyl, cyano, halogen, haloalkyl, heteroaryl, heterocyclyl, hydrogen, hydroxyl, nitro, sulfonamido, sulfonyl, and thio;
R₄ is selected from the group consisting of: alkyl, alkylsulfonyl, alkanoyl, cycloalkyl, haloalkyl, and hydrogen;
R₇ and R₈ are each independently selected from the group consisting of:
alkoxy, alkyl, halogen, haloalkyl, hydrogen and hydroxyl, or R₇ and R₈ taken together are oxo; or R₇ and R₈ taken together form a carbocycle or a heterocycle;
R₁₁ is selected from the group consisting of: alkyl, cycloalkyl, or haloalkyl wherein R₁₁ may optionally substituted by halogen, cyano, hydroxy, nitro, or alkoxy;
R₁₆ is selected from the group consisting of: alkyl, alkanoyl, cycloalkyl, haloalkyl, and hydrogen; and
R₁₄ and R₁₅ are each independently selected from the group consisting of: alkoxy, alkyl, alkanoyl, amino, aryl, cycloalkyl, cyano, halogen, haloalkyl, hydrogen, and hydroxyl.

97. A compound defined in claim 94 represented by formula II: 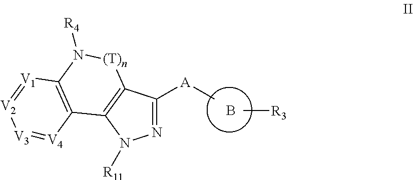
and pharmaceutically acceptable salts or N-oxides thereof; wherein
V₁, V₂, V₃, and V₄ are selected, independently for each occurrence, from the group consisting of: N and CR₂;
T is CO or CR₇R₈, wherein at least one T is CR₇R_{8;}
n is 0, 1 or 2;
A is selected from the group consisting of: —CONR₁₆—, —NR₁₆CO—, —O—, —NR₁₆CONR₁₆—, —CR₁₄R₁₅—NR₁₆CO—, —CR₁₄R₁₅—O—, —CR₁₄R₁₅—NR₁₆—, —O—CR₁₄R₁₅—, —NR₁₆—CR₁₄R₁₅—, —NR₁₆—, 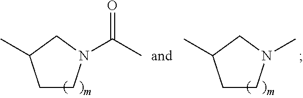
m is 0, 1 or 2;
R₂ is independently selected for each occurrence from the group consisting of: alkoxy, alkyl, alkenyl, alkynyl, alkanoyl, amino, aryl, carboxamido, cycloalkyl, cyano, halogen, haloalkyl, heteroaryl, heterocyclyl, hydrogen, hydroxyl, nitro, sulfonamido, sulfonyl, and thio;
B is selected from the group consisting of: aryl, heteroaryl, and heterocyclyl wherein B is optionally substituted by one, two, or three substituents each independently represented by R₃;
R₃ is selected from the group consisting of: alkoxy, alkyl, alkenyl, alkynyl, alkanoyl, amino, aryl, carboxamido, cycloalkyl, cyano, halogen, haloalkyl, heteroaryl, heterocyclyl, hydrogen, hydroxyl, nitro, sulfonamido, sulfonyl, and thio;
R₄ is selected from the group consisting of: alkyl, alkylsulfonyl, alkanoyl, cycloalkyl, haloalkyl, and hydrogen;
R₁₁ is selected from the group consisting of: alkyl, cycloalkyl, alkanoyl, aryl, heteroaryl, or heterocyclyl;
R₁₆ is selected from the group consisting of: alkyl, alkanoyl, cycloalkyl, haloalkyl, and hydrogen; and
R₁₄ and R₁₅ are each independently selected from the group consisting of:
alkoxy, alkyl, alkanoyl, amino, aryl, cycloalkyl, cyano, halogen, haloalkyl, hydrogen, and hydroxyl.

98. A compound defined in claim 94 represented by formula III: 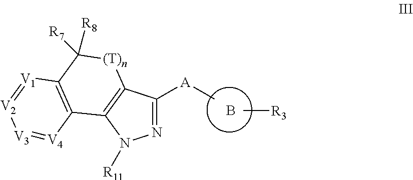
and pharmaceutically acceptable salts or N-oxides thereof; wherein
V₁, V₂, V₃, and V₄ are selected, independently for each occurrence, from the group consisting of: N and CR₂;
T is CO or CR₇R₈, wherein at least one T is CR₇R_{8;}
n is 0, 1 or 2;
A is selected from the group consisting of: —CONR₁₆—, —NR₁₆CO—, —NR₁₆CONR₁₆—, —O—, —CR₁₄R₁₅—NR₁₆CO—, —CR₁₄R₁₅—O—, —CR₁₄R₁₅—NR₁₆—, —O—CR₁₄R₁₅—, —NR₁₆—CR₁₄R₁₅—, —NR₁₆—, 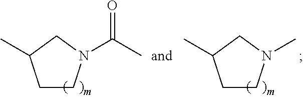
m is 0, 1 or 2;
R₂ is independently selected for each occurrence from the group consisting of: alkoxy, alkyl, alkenyl, alkynyl, alkanoyl, amino, aryl, carboxamido, cycloalkyl, cyano, halogen, haloalkyl, heteroaryl, heterocyclyl, hydrogen, hydroxyl, nitro, sulfonamido, sulfonyl, and thio;
B is selected from the group consisting of: aryl, heteroaryl, and heterocyclyl wherein B is optionally substituted by one, two, or three substituents each independently represented by R₃;
R₃ is selected from the group consisting of: alkoxy, alkyl, alkenyl, alkynyl, alkanoyl, amino, aryl, carboxamido, cycloalkyl, cyano, halogen, haloalkyl, heteroaryl, heterocyclyl, hydrogen, hydroxyl, nitro, sulfonamido, sulfonyl, and thio;
R₇ and R₈ are each independently selected from the group consisting of:
alkyl, halogen, haloalkyl, and hydrogen; or R₇ and R₈ taken together form a carbocycle or a heterocycle;
R₁₁ is selected from the group consisting of: alkyl, cycloalkyl, alkanoyl, aryl, heteroaryl, or heterocyclyl;
R₁₆ is selected from the group consisting of: alkyl, alkanoyl, cycloalkyl, haloalkyl, and hydrogen; and
R₁₄ and R₁₅ are each independently selected from the group consisting of: alkoxy, alkyl, alkanoyl, amino, aryl, cycloalkyl, cyano, halogen, haloalkyl, hydrogen, and hydroxyl.

99. A compound defined in claim 94 represented by formula IV: 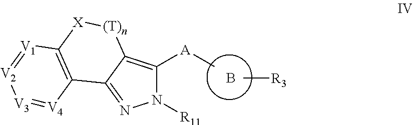
and pharmaceutically acceptable salts or N-oxides thereof; wherein
V₁, V₂, V₃, and V₄ are selected, independently for each occurrence, from the group consisting of: N and CR₂;
X is selected from the group consisting of: O, SO₂, NR₄ and CR₇R₈;
T is CO or CR₇R₈, wherein at least one T is CR₇R_{8;}
n is 0, 1 or 2;
A is selected from the group consisting of: —CONR₁₆—, —NR₁₆CO—, —O—, —NR₁₆CONR₁₆—, —CR₁₄R₁₅—NR₁₆CO—, —CR₁₄R₁₅—O—, —CR₁₄R₁₅—NR₁₆—, —O—CR₁₄R₁₅—, —NR₁₆—CR₁₄R₁₅—, —NR₁₆—, 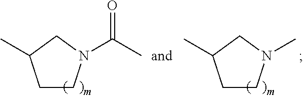
m is 0, 1 or 2;
R₂ is independently selected for each occurrence from the group consisting of: alkoxy, alkyl, alkenyl, alkynyl, alkanoyl, amino, aryl, carboxamido, cycloalkyl, cyano, halogen, haloalkyl, heteroaryl, heterocyclyl, hydrogen, hydroxyl, nitro, sulfonamido, sulfonyl, and thio;
B is selected from the group consisting of: aryl, heteroaryl, and heterocyclyl, wherein B is optionally substituted by one, two, or three substituents each independently represented by R₃;
R₃ is selected from the group consisting of: alkoxy, alkyl, alkenyl, alkynyl, alkanoyl, amino, aryl, carboxamido, cycloalkyl, cyano, halogen, haloalkyl, heteroaryl, heterocyclyl, hydrogen, hydroxyl, nitro, sulfonamido, sulfonyl, and thio;
R₄ is selected from the group consisting of: alkyl, alkylsulfonyl, alkanoyl, cycloalkyl, haloalkyl, and hydrogen;
R₇ and R₈ are each independently selected from the group consisting of:
alkoxy, alkyl, halogen, haloalkyl, hydrogen and hydroxyl, or R₇ and R₈ taken together are oxo; or R₇ and R₈ taken together form a carbocycle or a heterocycle;
R₁₁ is selected from the group consisting of: alkyl, cycloalkyl, or haloalkyl;
R₁₆ is selected from the group consisting of: alkyl, alkanoyl, cycloalkyl, haloalkyl, and hydrogen; and
R₁₄ and R₁₅ are each independently selected from the group consisting of:
alkoxy, alkyl, alkanoyl, amino, aryl, cycloalkyl, cyano, halogen, haloalkyl, hydrogen, and hydroxyl.

100. A compound defined in claim 94 represented by formula V: 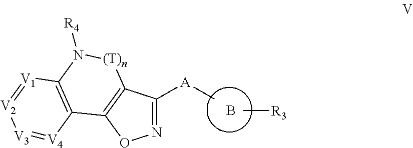
and pharmaceutically acceptable salts or N-oxides thereof; wherein
V₁, V₂, V₃, and V₄ are selected, independently for each occurrence, from the group consisting of: N and CR₂;
T is CO or CR₇R₈, wherein at least one T is CR₇R_{8;}
n is 0, 1 or 2;
A is selected from the group consisting of: —CONR₁₆—, —NR₁₆CO—, —O—, —NR₁₆CONR₁₆—, —CR₁₄R₁₅—NR₁₆CO—, —CR₁₄R₁₅—O—, —CR₁₄R₁₅—NR₁₆—, —O—CR₁₄R₁₅—, —NR₁₆—CR₁₄R₁₅—, —NR₁₆—, 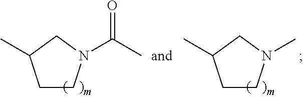
m is 0, 1 or 2;
R₂ is independently selected for each occurrence from the group consisting of: alkoxy, alkyl, alkenyl, alkynyl, alkanoyl, amino, aryl, carboxamido, cycloalkyl, cyano, halogen, haloalkyl, heteroaryl, heterocyclyl, hydrogen, hydroxyl, nitro, sulfonamido, sulfonyl, and thio;
B is selected from the group consisting of: aryl, heteroaryl, and heterocyclyl, optionally substituted by one, two or three substituents each independently represented by R₃;
R₃ is selected from the group consisting of: alkoxy, alkyl, alkenyl, alkynyl, alkanoyl, amino, aryl, carboxamido, cycloalkyl, cyano, halogen, haloalkyl, heteroaryl, heterocyclyl, hydrogen, hydroxyl, nitro, sulfonamido, sulfonyl, and thio;
R₄ is selected from the group consisting of: alkyl, alkylsulfonyl, alkanoyl, cycloalkyl, haloalkyl, and hydrogen;
R₇ and R₈ are each independently selected from the group consisting of:
alkyl, halogen, haloalkyl, and hydrogen; or R₇ and R₈ taken together are oxo; or R₇ and R₈ taken together form a carbocycle or a heterocycle;
R₁₆ is selected from the group consisting of: alkyl, alkanoyl, cycloalkyl, haloalkyl, and hydrogen; and
R₁₄ and R₁₅ are each independently selected from the group consisting of:
alkoxy, alkyl, alkanoyl, amino, aryl, cycloalkyl, cyano, halogen, haloalkyl, hydrogen, and hydroxyl.

101. A compound defined in claim 94 represented by formula VI: 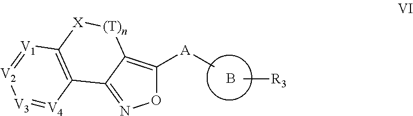
and pharmaceutically acceptable salts or N-oxides thereof; wherein
V₁, V₂, V₃, and V₄ are selected, independently for each occurrence, from the group consisting of: N and CR₂;
X is selected from the group consisting of: NR₄, S, SO₂, and CR₇R₈;
T is CO or CR₇R₈, wherein at least one T is CR₇R_{8;}
n is 0, 1 or 2;
A is selected from the group consisting of: —CONR₁₆—, —NR₁₆CO—, —O—, —NR₁₆CONR₁₆—, —CR₁₄R₁₅—NR₁₆CO—, —CR₁₄R₁₅—O—, —CR₁₄R₁₅—NR₁₆—, —O—CR₁₄R₁₅—, —NR₁₆—CR₁₄R₁₅—, —NR₁₆—, 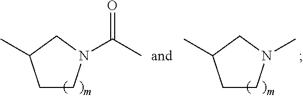
m is 0, 1 or 2;
R₂ is independently selected for each occurrence from the group consisting of: alkoxy, alkyl, alkenyl, alkynyl, alkanoyl, amino, aryl, carboxamido, cycloalkyl, cyano, halogen, haloalkyl, heteroaryl, heterocyclyl, hydrogen, hydroxyl, nitro, sulfonamido, sulfonyl, and thio;
B is selected from the group consisting of: aryl, heteroaryl, and heterocyclyl, wherein B is optionally substituted by one, two or three substituents each selected from R₃;
R₃ is selected from the group consisting of: alkoxy, alkyl, alkenyl, alkynyl, alkanoyl, amino, aryl, carboxamido, cycloalkyl, cyano, halogen, haloalkyl, heteroaryl, heterocyclyl, hydrogen, hydroxyl, nitro, sulfonamido, sulfonyl, and thio;
R₄ is selected from the group consisting of: alkyl, alkylsulfonyl, alkanoyl, cycloalkyl, haloalkyl, and hydrogen;
R₇ and R₈ are each independently selected from the group consisting of: alkyl, halogen, haloalkyl, and hydrogen, or R₇ and R₈ taken together are oxo; or R₇ and R₈ taken together form a carbocycle or a heterocycle;
R₁₁ is alkyl;
R₁₆ is selected from the group consisting of: alkyl, alkanoyl, cycloalkyl, haloalkyl, and hydrogen; and
R₁₄ and R₁₅ are each independently selected from the group consisting of:
alkoxy, alkyl, alkanoyl, amino, aryl, cycloalkyl, cyano, halogen, haloalkyl, hydrogen, and hydroxyl.

102. A pharmaceutical composition comprising at least compound according to claim 95 and a pharmaceutically acceptable carrier.

103. The method according to claim 94, wherein the compound is selected from the group consisting of:
N-(3-chlorophenyl)-1-methyl-4,5-dihydro-1H-benzo[g]indazole-3-carboxamide;
8-chloro-N-(3-chlorophenyl)-1-methyl-1,4-dihydrochromeno[4,3-c]pyrazole-3-carboxamide;
N-(3-chlorophenyl)-8-fluoro-2-methyl-2,4-dihydrochromeno[4,3-c]pyrazole-3-carboxamide;
N-(4-chloropyridin-2-yl)-1-methyl-1,4-dihydrochromeno[4,3-c]pyrazole-3-carboxamide;
N-(3-chlorophenyl)-8-fluoro-1-methyl-1,4-dihydrochromeno[4,3-c]pyrazole-3-carboxamide;
N-(3-chlorophenyl)-1,4,4-trimethyl-1,4-dihydrochromeno[4,3-c]pyrazole-3-carboxamide;
7-fluoro-1-methyl-N-(6-methylpyridin-2-yl)-1,4-dihydrochromeno[4,3-c]pyrazole-3-carboxamide;
3-chloro-N-((1-methyl-1,4-dihydrochromeno[4,3-c]pyrazol-3-yl)methyl)aniline;
1-methyl-N-(6-methylpyridin-2-yl)-4,5-dihydro-1H-pyrazolo[4,3-h]quinoline-3-carboxamide;
N-(6-methylpyridin-2-yl)-1,4-dihydrochromeno[4,3-c]pyrazole-3-carboxamide;
N-(4-cyanopyridin-2-yl)-1-methyl-1, 4-dihydrochromeno[4,3-c]pyrazole-3-carboxamide;
N-(2,3-dihydrobenzo[b][1,4]dioxin-6-yl)-1-methyl-1,4-dihydrochromeno[4,3-c]pyrazole-3-carboxamide;
(2H-benzo[b][1,4]oxazin-4(3H)-yl)(1-methyl-1,4-dihydrochromeno[4,3-c]pyrazol-3-yl)methanone;
5-acetyl-N-(3-chlorophenyl)-1-methyl-4,5-dihydro-1H-pyrazolo[4,3-c]quinoline-3-carboxamide;
N-(3-chlorophenyl)-1-methyl-1H-pyrazolo[4,3-c]quinoline-3-carboxamide;
N-(3-chlorophenyl)-1-methyl-4,5-dihydro-1H-pyrazolo[4,3-c]quinoline-3-carboxamide;
5-bromo-N-ethyl-N-((1-methyl-1,4-dihydrochromeno[4,3-c]pyrazol-3-yl)methyl)furan-2-carboxamide;
1-isopropyl-N-(6-methylpyridin-2-yl)-4,5-dihydro-1H-pyrazolo[4,3-h]quinoline-3-carboxamide;
1-ethyl-N-(6-methylpyridin-2-yl)-4,5-dihydro-1H-pyrazolo[4,3-h]quinoline-3-carboxamide;
1-methyl-N-(6-methylpyridin-2-yl)-1,4,5,6,7,8-hexahydro-7,8-(2-pyridino)cyclohepta[c]pyrazole-3-carboxamide;
1,8-dimethyl-N-(6-methylpyridin-2-yl)-4,5-dihydro-1H-pyrazolo[4,3-h]quinoline-3-carboxamide;
1-methyl-N-(6-methylpyridin-2-yl)-4,5-dihydro-1H-pyrazolo[4,3-h]isoquinoline-3-carboxamide;
1-methyl-N-(6-methylpyridin-2-yl)-4,5-dihydro-1H-pyrazolo[3,4-f]quinoline-3-carboxamide;
1,5-dimethyl-N-(6-methylpyridin-2-yl)-1,4,5,6-tetrahydropyrazolo[4,3-c]pyrido[2,3-e]azepine-3-carboxamide;
1,6-dimethyl-N-(6-methylpyridin-2-yl)-1,4,5,6-tetrahydropyrazolo[3,4-d]pyrido[3,2-b]azepine-3-carboxamide;
1-methyl-N-(6-methylpyridin-2-yl)-6-oxo-1,4,5,6-tetrahydrobenzo[e]pyrazolo[4,3-c]azepine-3-carboxamide;
1-methyl-N-(6-methylpyridin-2-yl)-5-oxo-1,4,5,6-tetrahydrobenzo[b]pyrazolo[3,4-d]azepine-3-carboxamide;
5,5-difluoro-1-methyl-N-(6-methylpyridin-2-yl)-4,5-dihydro-1H-pyrazolo[4,3-h]quinoline-3-carboxamide;
5,5-difluoro-1-methyl-N-(6-methylpyridin-2-yl)-4,5-dihydro-1H-benzo[g]indazole-3-carboxamide;
1,6-dimethyl-N-(6-methylpyridin-2-yl)-1,4,5,6-tetrahydrobenzo[b]pyrazolo[3,4-d]azepine-3-carboxamide;
1,5-dimethyl-N-(6-methylpyridin-2-yl)-4,5-dihydro-1H-pyrazolo[4,3-c]quinoline-3-carboxamide;
8-fluoro-1,5-dimethyl-N-(6-methylpyridin-2-yl)-4,5-dihydro-1H-pyrazolo[4,3-c]quinoline-3-carboxamide;
1,5-dimethyl-N-(6-methylpyridin-2-yl)-1,4,5,6-tetrahydrobenzo[e]pyrazolo[4,3-c]azepine-3-carboxamide;
1-methyl-N-(6-methylpyridin-2-yl)-5-oxo-4,5-dihydro-1H-pyrazolo[4,3-h]quinoline-3-carboxamide;
1-methyl-N-(6-methylpyridin-2-yl)-4H-benzo[b]imidazo[1,5-d][1,4]oxazine-3-carboxamide;
1-methyl-N-(6-methylpyridin-2-yl)-5-oxo-4,5-dihydro-1H-pyrazolo[4,3-h]quinoline-3-carboxamide;
1-methyl-N-(6-methylpyridin-2-yl)-4H-benzo[b]imidazo[1,5-d][1,4]oxazine-3-carboxamide;
1-methyl-N-(6-methylpyridin-2-yl)-5-oxo-4,5-dihydro-1H-pyrazolo[4,3-c]isoquinoline-3-carboxamide;
1,5-dimethyl-N-(6-methylpyridin-2-yl)-4,5-dihydro-1H-pyrazolo[4,3-c][1,5]naphthyridine-3-carboxamide;
9-fluoro-1-methyl-N-(6-methylpyridin-2-yl)-6-oxo-1,4,5,6-tetrahydrobenzo[e]pyrazolo[4,3-c]azepine-3-carboxamide;
1,5,5-trimethyl-N-(6-methylpyridin-2-yl)-4,5-dihydro-1H-pyrazolo[4,3-h]quinoline-3-carboxamide;
1-methyl-N-(6-(trifluoromethyl)pyridin-2-yl)-4,5-dihydro-1H-pyrazolo[4,3-h]quinoline-3-carboxamide;
1-methyl-N-(6-methylpyridin-2-yl)-4,5-dihydro-1H-pyrazolo[3,4-f]quinoxaline-3-carboxamide;
9-fluoro-1-methyl-N-(6-methylpyridin-2-yl)-4,5-dihydro-1H-pyrazolo[3,4-f]quinoline-3-carboxamide;
8-fluoro-1-methyl-N-(6-methylpyridin-2-yl)-4,5-dihydro-1H-pyrazolo[3,4-f]quinoline-3-carboxamide;
7-fluoro-1-methyl-N-(6-methylpyridin-2-yl)-4,5-dihydro-1H-pyrazolo[4,3-h]quinoline-3-carboxamide;
6-fluoro-1-methyl-N-(6-methylpyridin-2-yl)-4,5-dihydro-1H-pyrazolo[4,3-h]quinoline-3-carboxamide;
N-(6-methylpyridin-2-yl)-4H-benzo[b]imidazo[1,5-d][1,4]oxazine-3-carboxamide;
N-(6-cyclopropylpyridin-2-yl)-1-methyl-4,5-dihydro-1H-pyrazolo[4,3-h]quinoline-3-carboxamide;
1-methyl-N-(1-methyl-5-phenyl-1H-pyrrol-2-yl)-4,5-dihydro-1H-pyrazolo[4,3-h]quinoline-3-carboxamide;
6-methyl-N-(2-(1-methyl-4,5-dihydro-1H-pyrazolo[4,3-h]quinolin-3-yl)propan-2-yl)pyridin-2-amine;
3-(2-(3-chlorophenoxy)propan-2-yl)-1-methyl-4,5-dihydro-1H-pyrazolo[4,3-h]quinoline;
6-methyl-N-(1-methyl-4,5-dihydro-1H-pyrazolo[4,3-h]quinolin-3-yl)picolinamide;
3-(1-(3-chlorophenoxy)ethyl)-1-methyl-4,5-dihydro-1H-pyrazolo[4,3-h]quinoline;
6-cyclopropyl-N-(1-(1-methyl-4,5-dihydro-1H-pyrazolo[4,3-h]quinolin-3-yl)ethyl)pyridin-2-amine;
1-methyl-N-(6-methylpyridin-2-yl)-1,4,5,6-tetrahydro-6,7-(2-pyridino)cyclopenta[c]pyrazole-3-carboxamide;
1-methyl-N-(2-methylpyrimidin-4-yl)-4,5-dihydro-1H-pyrazolo[4,3-h]quinoline-3-carboxamide;
1-methyl-N-(6-methylpyrazin-2-yl)-4,5-dihydro-1H-pyrazolo[4,3-h]quinoline-3-carboxamide;
9-fluoro-1-methyl-N-(6-methylpyridin-2-yl)-5-oxo-1,4,5,6-tetrahydrobenzo[b]pyrazolo[3,4-d]azepine-3-carboxamide;
1-methyl-N-(6-methylpyridin-2-yl)-4,5,7,8-tetrahydrobenzo-1H-oxepino[4,5-c]pyrazole-3-carboxamide;
1-methyl-N-(6-methylpyridin-2-yl)-1,4,5,6-tetrahydrobenzo[e]pyrazolo[4,3-c]azepine-3-carboxamide;
1-methyl-N-(6-methylpyridin-2-yl)-4,5,7,8-tetrahydrobenzo-1H-thiepino[4,5-c]pyrazole-3-carboxamide;
1-methyl-N-(6-methylpyridin-2-yl)-6-dioxo-4,5,7,8-tetrahydro-7,8-(2-pyridino)-1H-thiepino[4,5-c]pyrazole-3-carboxamide;
1-methyl-3-(1-(6-methylpyridin-2-yl)azetidin-3-yl)-4,5-dihydro-1H-pyrazolo[4,3-h]quinoline;
1-methyl-N-(6-methylpyridin-2-yl)-4,5-dihydro-1H-pyrazolo[4,3-h]quinolin-3-amine;
1-methyl-N-(1-(6-methylpyridin-2-yl)ethyl)-4,5-dihydro-1H-pyrazolo[4,3-h]quinolin-3-amine;
1-methyl-3-(1-(6-methylpyridin-2-yl)ethoxy)-4,5-dihydro-1H-pyrazolo[4,3-h]quinoline;
1-methyl-N-(6-methylpyridin-2-yl)-4,5-dihydro-1H-pyrazolo[3,4-f]isoquinoline-3-carboxamide;
5-hydroxy-1-methyl-N-(6-methylpyridin-2-yl)-4,5-dihydro-1H-benzo[g]indazole-3-carboxamide;
1-methyl-N-(6-methylpyridin-2-yl)-1,4,5,6,7,8-hexahydro-6-hydroxybenzocyclohepta[c]pyrazole-3-carboxamide;
1-methyl-N-(6-methylpyridin-2-yl)-1,4,5,6,7,8-hexahydro-6,6-difluorobenzocyclohepta[c]pyrazole-3-carboxamide;
5-hydroxy-1-methyl-N-(6-methylpyridin-2-yl)-4,5-dihydro-1H-pyrazolo[4,3-h]quinoline-3-carboxamide;
1-methyl-N-(6-methylpyridin-2-yl)-1,4,5,6,7,8-hexahydro-6-hydroxy-7,8-(2-pyridino)cyclohepta[c]pyrazole-3-carboxamide;
1-methyl-N-(6-methylpyridin-2-yl)-1,4,5,6,7,8-hexahydro-6,6-difluoro-7,8-(2-pyridino)cyclohepta[c]pyrazole-3-carboxamide;
N-(3-chlorophenyl)-N-methyl-4H-chromeno[3,4-d]isoxazole-3-carboxamide;
1-methyl-N-(3-(trifluoromethyl)phenyl)-1,4-dihydrochromeno[4,3-c]pyrazole-3-carboxamide;
N-(3-chlorophenyl)-N,1-dimethyl-1,4-dihydrochromeno[4,3-c]pyrazole-3-carboxamide;
N-(2-chlorophenyl)-1-methyl-1,4-dihydrochromeno[4,3-c]pyrazole-3-carboxamide;
N-(4-chlorophenyl)-1-methyl-1,4-dihydrochromeno[4,3-c]pyrazole-3-carboxamide;
N-(4-chlorophenyl)-4,5-dihydro-1H-benzo[g]indazole-3-carboxamide;
N-(pyridin-3-yl)-1,4-dihydrochromeno[4,3-c]pyrazole-3-carboxamide;
N-(3-methoxyphenyl)-1,4-dihydrochromeno[4,3-c]pyrazole-3-carboxamide;
N-(4-methoxyphenyl)-1,4-dihydrochromeno[4,3-c]pyrazole-3-carboxamide;
N-(3-chlorophenyl)-1,8-dimethyl-1,4-dihydrothiochromeno[4,3-c]pyrazole-3-carboxamide;
N-(4-fluorophenyl)-1-methyl-1,4-dihydrochromeno[4,3-c]pyrazole-3-carboxamide;
N-(3-chloro-4-fluorophenyl)-1-methyl-1,4-dihydrochromeno[4,3-c]pyrazole-3-carboxamide;
N-(5-chloro-2-methoxyphenyl)-1-methyl-1,4-dihydrochromeno[4,3-c]pyrazole-3-carboxamide;
N-(3-methoxyphenyl)-1-methyl-1,4-dihydrochromeno[4,3-c]pyrazole-3-carboxamide;
N-(5-chloropyridin-2-yl)-1-methyl-1,4-dihydrochromeno[4,3-c]pyrazole-3-carboxamide;
N-(3-chlorophenyl)-1-methyl-1,4-dihydrothiochromeno[4,3-c]pyrazole-3-carboxamide;
N-(3-chlorobenzyl)-1-methyl-1,4-dihydrochromeno[4,3-c]pyrazole-3-carboxamide;
N-(3-chlorophenyl)-1-methyl-4,5-dihydro-1H-thieno[2,3-g]indazole-3-carboxamide;
N-(3-chlorophenyl)-4H-thiochromeno[3,4-d]isoxazole-3-carboxamide;
[1]Benzopyrano[4,3-c]pyrazole-3-carboxamide,1,4-dihydro-1-methyl-N-(6-methyl-2-pyridinyl)-;
[1]Benzopyrano[4,3-c]pyrazole-3-carboxamide, 1,4-dihydro-1-methyl-N-2-pyridinyl-;
[1]Benzopyrano[4,3-c]pyrazole-3-carboxamide, N-[3-(3-fluorophenyl)-5-isoxazolyl]-1,4-dihydro-1-methyl-;
[1]Benzopyrano[4,3-c]pyrazole-3-carboxamide, N-[3-(2-ethoxyphenyl)-5-isoxazolyl]-1,4-dihydro-1-methyl-;
[1]Benzopyrano[4,3-c]pyrazole-3-carboxamide, 1,4-dihydro-1-methyl-N-(5-methyl-2-pyridinyl)-;
[1]Benzopyrano[4,3-c]pyrazole-3-carboxamide, N-[3-(4-ethoxyphenyl)-5-isoxazolyl]-1,4-dihydro-1-methyl-;
[1]Benzopyrano[4,3-c]pyrazole-3-carboxamide, N-[3-(3-chlorophenyl)-5-isoxazolyl]-1,4-dihydro-1-methyl-;
[1]Benzopyrano[4,3-c]pyrazole-3-carboxamide, 1,4-dihydro-1-methyl-N-3-pyridinyl-;
1-methyl-N-(5-methylisoxazol-3-yl)-1,4-dihydrochromeno[4,3-c]pyrazole-3-carboxamide;
[1]Benzopyrano[4,3-c]pyrazole-3-carboxamide, N-(3,5-dimethylphenyl)-1,4-dihydro-1-methyl-;
Benzoic acid, 4-[[(1,4-dihydro-1-methyl[1]benzopyrano[4,3-c]pyrazol-3-yl)carbonyl]amino]-, methyl ester;
[1]Benzopyrano[4,3-c]pyrazole-3-carboxamide, N-[3-(3,4-diethoxyphenyl)-5-isoxazolyl]-1,4-dihydro-1-methyl-;
[1]Benzopyrano[4,3-c]pyrazole-3-carboxamide, N-[4-[(4-ethyl-1-piperazinyl)methyl]phenyl]-1,4-dihydro-1-methyl-;
[1]Benzopyrano[4,3-c]pyrazole-3-carboxamide, 1,4-dihydro-1-methyl-N-[4-[(4-methyl-1-piperidinyl)methyl]phenyl]-;
[1]Benzopyrano[4,3-c]pyrazole-3-carboxamide, 1,4-dihydro-1-methyl-N-[4-(1-piperidinylsulfonyl)phenyl]-;
[1]Benzopyrano[4,3-c]pyrazole-3-carboxamide, N-[4-[(diethylamino)sulfonyl]phenyl]-1,4-dihydro-1-methyl-;
1,3-Benzodioxole-5-carboxylic acid, 6-[[(1,4-dihydro-1-methyl[1]benzopyrano[4,3-c]pyrazol-3-yl)carbonyl]amino]-, methyl ester;
[1]Benzopyrano[4,3-c]pyrazole-3-carboxamide, 1,4-dihydro-1-methyl-N-(4-methyl-2-oxo-2H-1-benzopyran-7-yl)-;
2-Thiophenecarboxylic acid, 3-[[(1,4-dihydro-1-methyl[1]benzopyrano[4,3-c]pyrazol-3-yl)carbonyl]amino]-, methyl ester;
[1]Benzopyrano[4,3-c]pyrazole-3-carboxamide, N-(2,5-difluorophenyl)-1,4-dihydro-1-methyl-;
[1]Benzopyrano[4,3-c]pyrazole-3-carboxamide, N-(3-acetylphenyl)-1,4-dihydro-1-methyl-;
Benzoic acid, 4-[[(1,4-dihydro-1-methyl[1]benzopyrano[4,3-c]pyrazol-3-yl)carbonyl]amino]-, ethyl ester;
[1]Benzopyrano[4,3-c]pyrazole-3-carboxamide, N-[4-(aminosulfonyl)phenyl]-1,4-dihydro-1-methyl-;
[1]Benzopyrano[4,3-c]pyrazole-3-carboxamide, N-(4-chlorophenyl)-1,4-dihydro-1-methyl-;
[1]Benzopyrano[4,3-c]pyrazole-3-carboxamide, N-(2-chlorophenyl)-1,4-dihydro-1-methyl-;
[1]Benzopyrano[4,3-c]pyrazole-3-carboxamide, N-(2-cyanophenyl)-1,4-dihydro-1-methyl-;
N-(2-carbamoylphenyl)-1-methyl-1,4-dihydrochromeno[4,3-c]pyrazole-3-carboxamide;
[1]Benzopyrano[4,3-c]pyrazole-3-carboxamide, 1,4-dihydro-N-(4-methoxyphenyl)-1-methyl-;
[1]Benzopyrano[4,3-c]pyrazole-3-carboxamide, N-(2-chloro-3-pyridinyl)-1,4-dihydro-1-methyl-;
[1]Benzopyrano[4,3-c]pyrazole-3-carboxamide, N-(4-acetylphenyl)-1,4-dihydro-1-methyl-;
1,3-Benzenedicarboxylic acid, 5-[[(1,4-dihydro-1-methyl[1]benzopyrano[4,3-c]pyrazol-3-yl)carbonyl]amino]-, 1,3-dimethyl ester;
[1]Benzopyrano[4,3-c]pyrazole-3-carboxamide, N-(2-ethylphenyl)-1,4-dihydro-1-methyl-;
[1]Benzopyrano[4,3-c]pyrazole-3-carboxamide, 1,4-dihydro-1-methyl-N-(2-methylphenyl)-;
[1]Benzopyrano[4,3-c]pyrazole-3-carboxamide, 1,4-dihydro-1-methyl-N42-(4-methyl-1-piperazinyl)phenyl]-;
Benzoic acid, 4-[[(1,4-dihydro-1-methyl[1]benzopyrano[4,3-c]pyrazol-3-yl)carbonyl]amino]-, 2-(diethylamino)ethyl ester;
[1]Benzopyrano[4,3-c]pyrazole-3-carboxamide, N-(5-chloro-2-pyridinyl)-1,4-dihydro-1-methyl-;
[1]Benzopyrano[4,3-c]pyrazole-3-carboxamide, 1,4-dihydro-1-methyl-N-[3-(methylthio)phenyl]-;
[1]Benzopyrano[4,3-c]pyrazole-3-carboxamide, N-(3-ethylphenyl)-1,4-dihydro-1-methyl-;
[1]Benzopyrano[4,3-c]pyrazole-3-carboxamide, N-(9-ethyl-9H-carbazol-3-yl)-1,4-dihydro-1-methyl-;
[1]Benzopyrano[4,3-c]pyrazole-3-carboxamide, 1,4-dihydro-1-methyl-N-(2-methyl-3-nitrophenyl)-;
[1]Benzopyrano[4,3-c]pyrazole-3-carboxamide, N-(2-chloro-4-methylphenyl)-1,4-dihydro-1-methyl-;
[1]Benzopyrano[4,3-c]pyrazole-3-carboxamide, N-(4-chloro-2-methoxy-5-methylphenyl)-1,4-dihydro-1-methyl-;
[1]Benzopyrano[4,3-c]pyrazole-3-carboxamide, 1,4-dihydro-1-methyl-N-[4-(1-methylethyl)phenyl]-;
[1]Benzopyrano[4,3-c]pyrazole-3-carboxamide, N-(4-ethylphenyl)-1,4-dihydro-1-methyl-;
[1]Benzopyrano[4,3-c]pyrazole-3-carboxamide, 1,4-dihydro-N-(2-methoxy-5-methylphenyl)-1-methyl-;
[1]Benzopyrano[4,3-c]pyrazole-3-carboxamide, N-(2-ethoxyphenyl)-1,4-dihydro-1-methyl-;
Benzoic acid, 2-[[(1,4-dihydro-1-methyl[1]benzopyrano[4,3-c]pyrazol-3-yl)carbonyl]amino]-, ethyl ester;
[1]Benzopyrano[4,3-c]pyrazole-3-carboxamide, 1,4-dihydro-N-(2-methoxy-5-nitrophenyl)-1-methyl-;
[1]Benzopyrano[4,3-c]pyrazole-3-carboxamide, N-(2,5-dimethoxyphenyl)-1,4-dihydro-1-methyl-;
[1]Benzopyrano[4,3-c]pyrazole-3-carboxamide, N-(4-ethoxyphenyl)-1,4-dihydro-1-methyl-;
Benzoic acid, 2-[[(1,4-dihydro-1-methyl[1]benzopyrano[4,3-c]pyrazol-3-yl)carbonyl]amino]-, methyl ester;
[1]Benzopyrano[4,3-c]pyrazole-3-carboxamide, N-(2,3-dihydro-1,4-benzodioxin-6-yl)-1,4-dihydro-1-methyl;
[1]Benzopyrano[4,3-c]pyrazole-3-carboxamide, N-(4-bromo-2-methylphenyl)-1,4-dihydro-1-methyl-;
[1]Benzopyrano[4,3-c]pyrazole-3-carboxamide, N-(5-chloro-2,4-dimethoxyphenyl)-1,4-dihydro-1-methyl-;
[1]Benzopyrano[4,3-c]pyrazole-3-carboxamide, 1,4-dihydro-1-methyl-N-(3,4,5-trimethoxyphenyl)-;
[1]Benzopyrano[4,3-c]pyrazole-3-carboxamide, 1,4-dihydro-N-(3-methoxyphenyl)-1-methyl-;
[1]Benzopyrano[4,3-c]pyrazole-3-carboxamide, N-(2-fluorophenyl)-1,4-dihydro-1-methyl-;
[1]Benzopyrano[4,3-c]pyrazole-3-carboxamide, N-(5-chloro-2-methoxyphenyl)-1,4-dihydro-1-methyl-;
[1]Benzopyrano[4,3-c]pyrazole-3-carboxamide, N-(2,4-difluorophenyl)-1,4-dihydro-1-methyl-;
[1]Benzopyrano[4,3-c]pyrazole-3-carboxamide, N-(2,4-dimethoxyphenyl)-1,4-dihydro-1-methyl-;
[1]Benzopyrano[4,3-c]pyrazole-3-carboxamide, N-(3-chloro-4-methoxyphenyl)-1,4-dihydro-1-methyl-;
[1]Benzopyrano[4,3-c]pyrazole-3-carboxamide, N-(3-chloro-4-fluorophenyl)-1,4-dihydro-1-methyl-;
[1]Benzopyrano[4,3-c]pyrazole-3-carboxamide, N-(2-bromo-4-methylphenyl)-1,4-dihydro-1-methyl-;
[1]Benzopyrano[4,3-c]pyrazole-3-carboxamide, 1,4-dihydro-1-methyl-N-[3-(trifluoromethyl)phenyl]-;
[1]Benzopyrano[4,3-c]pyrazole-3-carboxamide, N-(3-chlorophenyl)-1,4-dihydro-1-methyl-;
[1]Benzopyrano[4,3-c]pyrazole-3-carboxamide, N-(4-fluorophenyl)-1,4-dihydro-1-methyl-;
[1]Benzopyrano[4,3-c]pyrazole-3-carboxamide, 1,4-dihydro-N-(2-methoxyphenyl)-1-methyl-;
and pharmaceutically acceptable salts or N-oxides thereof.
